SEQUENCE LISTING (19 sequences)

SEQ ID NO: 1 - protein, 34 aa, Artificial Sequence
  Synthetic peptide
PRKGGRRRRAWGRRRRRRRRRRRRRRAPRKRLTL

SEQ ID NO: 2 - protein, 33 aa, Artificial Sequence
  Synthetic peptide
YGRKKRRQRRRYGRKKRRQRRRYGRKKRRQRRR

SEQ ID NO: 3 - protein, 18 aa, Artificial Sequence
  Synthetic peptide
RRRRRRRRRRRRRRRRRR

SEQ ID NO: 4 - protein, 31 aa, Artificial Sequence
  Synthetic peptide
HHHHHHHHHHHHHHHHRRRRRRRRRRRRRRR

SEQ ID NO: 5 - protein, 15 aa, Artificial Sequence
  Synthetic peptide
RRRRRRRRRRRRRRR

SEQ ID NO: 6 - protein, 14 aa, Artificial Sequence
  Synthetic peptide
RRRRRRRRRRRRRR

SEQ ID NO: 7 - protein, 14 aa, Artificial Sequence
  Synthetic peptide
RRRRRRRRRRRRGC

SEQ ID NO: 8 - protein, 37 aa, Artificial Sequence
  Synthetic peptide
SGRGKQGGKARAKAKTRSSRAGLQFPVGRVHRLLRKG

SEQ ID NO: 9 - protein, 11 aa, Artificial Sequence
  Synthetic peptide
RRRRRRRRRRR

SEQ ID NO: 10 - protein, 14 aa, Artificial Sequence
  Synthetic peptide
RNRSRRRRNRSRRR

SEQ ID NO: 11 - protein, 17 aa, Artificial Sequence
  Synthetic peptide
TRQARRNRRRRWRERQR

SEQ ID NO: 12 - protein, 8 aa, Artificial Sequence
  Synthetic peptide
RRRRRRRR

SEQ ID NO: 13 - protein, 9 aa, Artificial Sequence
  Synthetic peptide
RRRRRRRRR

SEQ ID NO: 14 - protein, 11 aa, Artificial Sequence
  Synthetic peptide
YGRRRRRRRRR

SEQ ID NO: 15 - protein, 11 aa, Artificial Sequence
  Synthetic peptide
GGRRARRRRRR

SEQ ID NO: 16 - protein, 11 aa, Artificial Sequence
  Synthetic peptide
YGRRARRRRRR

SEQ ID NO: 17 - protein, 19 aa, Artificial Sequence
  Synthetic peptide
RLLRLLRRLLRLLRRLLRC

SEQ ID NO: 18 - protein, 25 aa, Artificial Sequence
  Synthetic peptide
ARRRRCSGSGSGCGSGSGSCGRRRR

SEQ ID NO: 19 - protein, 6 aa, Artificial Sequence
  Synthetic peptide
RRRRRR

(19) United States
(12) Patent Application Publication
(10) Pub. No.: US 2020/0031873 A1
(43) Pub. Date: Jan. 30, 2020

(54) REPURPOSING CELL PENETRATING PEPTIDES AND THEIR NOVEL DERIVATIVES AND IOPROMIDE AND IODO-ARYL CARBONATES FOR TREATMENT OF SENESCENCE-RELATED DISEASES AND DISORDERS

(71) Applicant: Eternans Limited, Esher (GB)

(72) Inventors: Süleyman Selim Çinarouglu, Istanbul (TR); Emel Timucin, Istanbul (TR); Gunseli Bayram Akcapinar, Istanbul (TR); Ugur Sezerman, Istanbul (TR); Yavuz Ahiska, Esher (GB)

(21) Appl. No.: 16/526,997

(22) Filed: Jul. 30, 2019

Related U.S. Application Data
(60) Provisional application No. 62/712,031, filed on Jul. 30, 2018.

(51) Int. Cl. C07K 14/00; C07K 7/64

(52) U.S. Cl. CPC C07K 14/001; A61K 38/00; C07K 7/64

(57) ABSTRACT

Methods of use for selected cell penetrating peptides (CPPs) and iopromide repurposed as senolytic agents for treating senescence-associated diseases and disorders by specifically targeting the FoxO4 protein. The diseases and disorders treatable with said repurposed senolytic agents include diseases with inflammatory origin including but not restricted to diabetes, cardiovascular diseases, or pulmonary diseases, including COPD; asthma, emphysema, or breathlessness; renal or hepatic insufficiency, cirrhosis, or osteoarthritis; senescence-associated ophthalmic diseases and disorders; and senescence-associated dermatological diseases and disorders; diabetic ulcers; kyphosis; scoliosis; weight loss; hair loss; muscle loss; loss of bone density; frailty and/or reduced fitness; hearing loss such as deafness; or combinations thereof.

### CROSS-REFERENCE TO RELATED APPLICATIONS

This application claims priority to U.S. Application Serial No. U.S. 62/712,031 filed Jul. 30, 2018 “REPURPOSING CELL PENETRATING PEPTIDES AND THEIR NOVEL DERIVATIVES AND IOPROMIDE AND IODO-ARYL CARBONATES FOR TREATMENT OF SENESCENCE-RELATED DISEASES AND DISORDERS,” which is incorporated herein in its entirety.

### REFERENCE TO SEQUENCE LISTING

The content of the ASCII text file of the sequence listing named “5442_20_SEQ_LISTING_ST25.txt” which is 6 KB in size and was created on Jul. 30, 2019 and included herewith is incorporated herein by reference in its entirety.

### BACKGROUND

Senescence and apoptosis are among mechanisms that, when activated, restrict tumor growth. Through apoptosis, damaged cells are cleared from the organism while senescent cells remain alive in the organism though permanently restricted from entering the cell cycle. Senescence may be associated with an increase in metabolic activity. In the majority of cases, senescent cells develop a defined, but heterogeneous, secretory profile termed as senescence-associated secretory phenotype (SASP). The SASP entails release of pro-inflammatory cytokines and chemokines, tissue-damaging proteases, factors that can affect stem and progenitor cell function, haemostatic factors, and growth factors, among others. Senescent cells that express the SASP can have substantial local and systemic pathogenic effects.

The SASP secretion comprises a range of different proteins, including several proteins known to play a role in aging and age-related diseases, including matrix metalloproteases such as MMP3, growth factors, chemokines such as CCL2 and CLL11, and prominent interleukins (ILs) such as IL1, IL6, and IL8. Above a certain threshold, such factors can significantly impair tissue function. The chronic SASP secretion by senescent cells may impair the functioning of neighboring cells. As such, senescent cells are thought to be major contributors of inflammation. Theoretically, low, but chronic, levels of inflammation are drivers of age-related decline in function. Consistent with this theory, senescence and SASP are elevated in a number of fast-aging mouse models and, where tested, senescence clearance delays their decline in health.

Additionally, mutations occurring in the senescent cells may lead to changes that allow those senescent cells to escape from cell cycle arrest and eventually be convert to tumorigenic cells. Thus senescent cells that display resistance to apoptosis and accumulate with age are targets of anti-aging research. The main aim of such research has been to discover the molecular pathways that direct cells to senescence but not apoptosis and, eventfully, to develop agents that interfere with these pathways so that administration of such agents will induce apoptosis in a senescent cell in a safe and predictable manner.

The forkhead box (FOX) protein family comprises the FOX class O subfamily (FoxO) that has 4 mammalian members: Forkhead box protein O1 (FoxO1) (FKHR, FoxO1A), Forkhead box protein O3 (FoxO3) (FKHRL1, FoxO3A), Forkhead box protein 04 (FoxO4) (AFX, AFX1, MLLT7), and Forkhead box protein O6 (FoxO6). FoxOs are transcription factors that play important roles in suppression of tumors. Despite the fact that the exact roles of FoxOs in senescence remain to be unraveled, it has been well-established for the FoxO4 that its mRNA and protein levels have been specifically increased in response to genotoxic activation of senescence. In line with this observation, FoxO4 function was essential for senescence activation by genotoxic damage, while the loss of FoxO4 function was associated with the apoptosis of senescent cells. Hence FoxO4 has a pivotal role in the cells' direction to either senescence or apoptosis. Interfering with this key molecule FoxO4 represents efficient way of blocking senescence and steering the cells' fate towards apoptosis.

Senescence has been characterized to be a complex phenomenon that can be activated by distinct signals. In particular, genotoxic activation of senescence has been characterized by the formation of DNA-SCARS (DNA Segments with Chromatin Alterations Reinforcing Senescence). When genotoxic damage is present, FoxO4 is recruited to DNA-SCARS that also includes tumor protein 53 (p53) as a major component. Oncogenic BRAF mutation at V600E are encountered in ˜7% of all human tumors with particularly enhanced occurrence in melanoma (˜70%). Additionally, melanoma cells were found to generally have an elevated number of DNA-SCARS containing FOXO4. It has been shown that the BRAF mutation promotes the FoxO4 leading to senescence, while at the same time it also causes phosphorylation of Serine 46 (S46) of p53, the condition which was known to strongly favor apoptosis over senescence. It has been shown for the DNA-damaged melanoma cells that interference with their FoxO4 expression results in a marked increase of apoptosis. Overall these findings pointed out the FoxO4 presence inhibited the apoptosis by the S46 phosphorylated p53. Furthermore, it has been shown that inhibition of the kinase that phosphorylates the S46 of p53 led to impaired apoptosis of the senescent cells even in the absence of FoxO4. These particular observations proposed a pivotal role for FoxO4 in restraining the apoptosis mediated by the S46 phosphorylated p53. Therefore, eradicating the senescence and inducing the apoptosis via blocking the action of FoxO4 on p53 is of interest, as discussed herein.

FoxO4 inhibition for senolytic therapy using naturally occurring peptide sequences interferes with the transcriptional activity of p53 and thus lead to unwanted side effects including tumor growth. Additionally, peptide sequences entirely derived from the human FoxO4 protein, will maintain very similar characteristics to the endogenous FoxO4 such as DNA binding and thus these peptides will also interfere with the function of FoxOs.

It is well established that chronic inflammation is the cause of many diseases. As stated above, SASP may contribute to chronic inflammation in old age. However, there is no generic senolytic agent, but a number of drugs with separate Senescent Cell Anti-apoptotic Pathways (SCAPs) and cell types some of which are Dasatinib (which acts on Dependence receptor/Src kinase/tyrosine kinase and target Primary human and mouse preadipocytes (adipose-derived stem cells), Quercetin (which acts on Bcl-2 family, p53/p21/serpine, & PI3K/AKT and target HUVECs, mouse bone marrow-derived mesenchymal stem cells), Navitoclax (which acts on ABT263 and target MR-90 Cells, HUVECs), Piperlongumin (which acts on A1331852/A1155463 and target IMR-90 Cells, HUVECs), and Fisetin (which acts on PI3K/AKT and targets HUVECs). The molecular pathway of the Senolytic Peptides disclosed herein are different than those mentioned in this paragraph therefore the methods of use of these Senolytic Peptide(s) for said diseases and disorders are different.

It is well established that chronic inflammation is the cause of many diseases. As stated above SASP is the primary cause of chronic inflammation in old age. US patent application US20160339019A1 collates the list of chronic inflammation caused by senescent associated diseases and disorders and proposes how a generic senolytic agent is administered for said diseases and disorders. Nevertheless senescence can be caused by multiple separate Senescent Cell Anti-apoptotic Pathways (SCAPs) partly depending on cell types and therefore there could not be a single generic senolytic agent but a number of different agents interfering with each of these separate SCAPs. Some of the drugs which are disclosed in prior art as senolytic agents are Dasatinib, which acts on Dependence receptor/Src kinase/tyrosine kinase and target Primary human and mouse preadipocytes (adipose-derived stem cells); Quercetin, which acts on Bcl-2 family, p53/p21/serpine, & PI3K/AKT and target HUVECs, mouse bone marrow-derived mesenchymal stem cells; Navitoclax, which acts on ABT263) and target MR-90 Cells, HUVECs and Piperlongumin which acts on A1331852/A1155463 and target IMR-90 Cells, HUVECs; and Fisetin which acts on PI3K/AKT and targets HUVECs. The inventive subject matter is a list of selected CPPs and iopromide which act on a particular molecular SCAP, which is different than those mentioned herein this paragraph.

CPPs are discussed in a number of patent applications including U.S. Pat. No. 8,575,305B2, U.S. Pat. No. 8,372,951B2, U.S. Pat. No. 8,044,019B2, WO2014053622A1, and US20130164219A1. Polyarginine CPPs are discussed in the patent applications US20120121670A1 and WO2005032593A1. None of the disclosed information in the references cover the use of CPPs as senolytic agents. All of these applications are related to the use of CPPs as intracellular delivery agents (delivery vehicles). The inventive subject matter is the repurposed use of CPPs and their novel derivatives for treatments of senescence-associated diseases and disorders which is completely different than the purpose and method of use described in the aforementioned patents within this paragraph.

### SUMMARY OF THE INVENTION

Disclosed herein are cell penetrating peptides (CPPs), referred to as the “Senolytic Peptides,” including those peptides included as a part of this application in the SEQUENCE LISTING as SEQ ID NO: 1 through SEQ ID NO: 19 which are effective to induce apoptosis of senescent cells in a subject, such as a mammal, by inhibiting the action of FoxO4 on p53. In some embodiments, the Senolytic Peptides disclosed herein are unnatural peptide(s) that are optimized to maximize their interference with the FoxO4, which is up-regulated in senescent cells. Additionally or alternatively, in some embodiments, the Senolytic Peptides may be designed to effectively block the CR3 domain of FoxO4 from interfering with the DNA binding function of p53 that is phosphorylated at Serine 46, particularly, from interfering with the bulky FH domain of FoxO4. Additionally or alternatively, in some embodiments the Senolytic Peptides may be rationally designed to minimize their interaction with the DBD of p53, other FoxOs, and the DNA duplex containing a FoxO consensus binding. Compared to the prior art, the Senolytic Peptides present a safer inhibitor of FoxO4 with minimal side effects resultant from interferences from p53DBD, other FoxOs and DNA.

Selectively killing a senescent cell in a mammal is a rejuvenation therapy particularly for post-production adult human cohort. It enables treatment of senescence-related diseases and disorders. The Associated Disclosed Material 1 disclosed the first structural model of the FoxO4-p53 complex which unraveled the inhibitory action of the CR3 domain of FoxO4 on the p53. Thus by blocking the CR3 domain of FoxO4, the inhibitory action of FoxO4 on p53 would be eliminated and p53 will be released from the FoxO4 complex to function in the apoptotic pathways.

One aspect of the inventive subject matter described in this patent application discloses a novel method of use for the 19 different CPP sequences listed in Table 1 as senolytic agents for treatment of senescence-associated diseases. These listed CCPs are normally used as intracellular delivery agents. After extensive in-silico analysis, these peptides were discovered to target the CR3 domain of FoxO4, thus said CPPs are selected to be suitable to target senescent cells by blocking FoxO4-p53 complex, and induce them to apoptosis.

Other aspects of the inventive subject matter of this application is the disclosure of additional novel peptides which are derived from the listed sequences in the Table 1 and are described under the heading of “Senolytic Cell Penetrating Peptides for the Treatment of Senescence Associated Disorders”. Not intending to be bound by theory, our extensive in silico analysis suggested that these selected 19 CPPs are able to interfere with SCAP by targeting the CR3 domain of FoxO4, due to the positively charged amino acids found in CPP sequences. The novel mutant derivatives of said CPPs containing at least 70% of the native CPP sequence or formed by repetition of deletion of any part of the CPP sequences will still be able to interfere with CR3 domain of FoxO4 thereby interfering with SCAP. From hereon the senolytic CPPs definition hereafter shall include those CPPs listed in Table 1 and their said novel derivatives. From hereon, CPPs of Table 1 and their novel derivatives will be jointly or separately referred as “senolytic CPPs”, wherein senolytic CPPs are repurposed for senescence associated diseases and disorders, wherein the novel derivatives of CPPs of Table 1 are obtained via mutagenesis and/or shuffling of any part of the sequence of the CPPs of Table 1 that are at least 6 amino acid in length, wherein novel CPP derivates contain unnatural amino acids and/or cyclic backbone and/or any modifications to the N-terminus and/or, C-terminus and/or inner sequence.

Herein this patent application the senolytic CPPs shall be called “Senolytic Peptides.”

These Senolytic Peptides do not have to be continuously present to exert their effects. Brief disruption of FoxO4 expressed pro-survival pathways is adequate to kill senescent cells. The inventive treatment specifies that Senolytic Peptides are administered intermittently. The Senolytic Peptides trigger apoptosis of the senescent cells which intern stimulate overall rejuvenation in a safe manner by reactivating the stem cell.

A method is disclosed herein for selectively killing senescent cells and for treating senescence-associated diseases and disorders by administering these Senolytic Peptides to minimize the interaction between FoxO4 and p53. The method of use of the novel senolytic agents comprised three different dose regimes and their variations, wherein each dose regime is expressed by distinct therapeutically effective amount and distinct administration frequencies. Each said dose regime is developed to treat different types of senescence-associated diseases and disorders. Killing senescent cells reduces the inflammatory SASP, and therefore reduces the chronic inflammation in the metabolism. Further, said treatment stimulates overall rejuvenation in a safe manner. The diseases and disorders treatable with said Senolytic Peptides include all diseases with inflammatory origin including diabetes, cardiovascular diseases, pulmonary diseases, osteoarthritis; senescence-associated ophthalmic diseases and disorders; and senescence-associated dermatological diseases and disorders. The applicable regimes and individualization of the treatment for said diseases are described under corresponding headings within this application.

In some embodiments, a method for selectively inducing apoptosis of senescent cells and/or for treating a senescence-associated disease or disorder comprises administering one or more of the Senolytic Peptides which minimize the interaction between FoxO4 and p53. In various embodiments, the method may comprise administering one or more of the Senolytic Peptide(s) via a treatment regime as disclosed herein or some variation thereof. In various embodiments, treatment regimes may vary as to administration frequency and/or dosing (for example, as to the a dosage that will be therapeutically effective). Particular treatment regimes may be specifically developed to treat different types of senescence-associated diseases or disorders. Induction of apoptosis in senescent cells (that is, killing senescent cells) reduces the inflammatory senescence-associated secretory phenotype and therefore significantly reduce the chronic inflammation in the metabolism. Further, said treatment may stimulate overall rejuvenation in a safe manner. Thus, the diseases and disorders treatable via the Senolytic Peptides may include, but not limited to, all diseases with inflammatory origin including diabetes, cardiovascular diseases, pulmonary diseases, osteoarthritis; senescence-associated ophthalmic diseases and disorders; and senescence-associated dermatological diseases and disorders. The applicable regimes and individualization of the treatment for said diseases are presented under corresponding headings within this application.

In some embodiments, the Senolytic Peptides may not have to be continuously present to exert an effect. For examples, brief disruption of pro-survival pathways, such as by administration of a Senolytic Peptide, is adequate to kill senescent cells. Thus, in some embodiments, the Senolytic Peptides are suitable to be administered intermittently.

It is to be understood that both the foregoing general description and the following detailed description are exemplary and explanatory only and are not restrictive of the invention, as claimed.

### BRIEF DESCRIPTION OF THE DRAWINGS

FIG. 1. Multiple alignment of the CR3 domain of FoxOs.

FIGS. 2A-2R. The CR3 domain interactions with the selected peptides listed in the Table 1. The snapshots were taken from the last 1 ns of MD simulations.

FIG. 3A illustrates the dose in which the Senolytic Peptides shall be administered following guidance provided in the Impulse Regime. The therapeutically Effective Dose and frequency of administration may vary within this regime. The Impulse Regime presents an optional follow up treatment course in which the therapeutically Effective Dose may be increased or decreased. FIG. 3A depicts at least six different considerations: (1) Treatment Cycle (comprised of subject's initial assessment, administration of therapeutically Effective Dose and final assessment); (2) therapeutically Effective Dose delivered in 1, 2, or 3 days; (3) Optional further treatment cycle(s) may be required if senescent cell reduction is unsatisfactory; (4) Senescence Clearance Interval 2-4 weeks; (5) Initial senescence assessment via biopsy samples; and (6) Final senescence assessment via biopsy samples.

FIG. 3B illustrates the dose in which the Senolytic Peptides shall be administered following guidance provided in the Sustained Regime. The therapeutically Effective Dose and frequency of administration may vary within this regime. The Sustained Regime presents an optional follow up treatment course in which the therapeutically Effective Dose may be increased or decreased. FIG. 3B depicts: (1) Treatment Cycle (comprised of subject's initial assessment, administration of therapeutically Effective Dose and final assessment); (2) therapeutically Effective Dose delivered in 1, 2, or 3 weeks; (3) Optional further treatment cycle(s) may be required if senescent cell reduction is unsatisfactory. therapeutically Effective Dose may be readjusted; (4) Senescence Clearance Interval 2-4 weeks; (5) Initial senescence assessment via biopsy samples; and (6) Final senescence assessment via biopsy samples.

FIG. 3C illustrates the dose in which the Senolytic Peptides shall be administered following guidance provided in the Gentle Regimes. The therapeutically Effective Dose and frequency of administration may vary within this regime. The Gentle Regime presents an optional follow up treatment course in which the therapeutically Effective Dose may be increased or decreased. FIG. 3C depicts: (1) Treatment Cycle (comprised of subject's initial assessment, administration of therapeutically Effective Dose and final assessment); (2) therapeutically Effective Dose delivered in 3 or 3 weeks; (3) Optional further treatment cycle(s) may be required if senescent cell reduction is unsatisfactory. therapeutically Effective Dose may be readjusted; (4) Senescence Clearance Interval 2-4 weeks; (5) Initial senescence assessment via biopsy samples; and (6) Final senescence assessment via biopsy samples.

FIG. 4A illustrates a selective decrease in the viability of senescent cells but not non-senescent cells resulting from administration of a Senolytic Peptide (SEQ ID NO: 2).

FIG. 4B illustrates the senolytic effect of a Senolytic Peptide (SEQ ID NO: 2) superior to FoxO4-DRI as shown by a sharper decrease in viability curves of the senescent IMR90 cells.

FIG. 5A illustrates a selective decrease in the viability of senescent cells but not non-senescent cells resulting from administration of a senolytic Peptide (SEQ ID NO: 11).

FIG. 5B illustrates the senolytic effect of the Senolytic Peptide (SEQ ID NO: 2) superior to FoxO4-DRI as shown by a sharper decrease in viability curves of the senescent WI-38 cells.

FIG. 6 illustrates the results of the evaluation of the senolytic Peptides for toxicity according to Acute Sytemic Toxicity Test (ISO 10993-11).

### DETAILED DESCRIPTION OF THE INVENTION

With respect to the following disclosure, the following definitions apply:

“Adjuvant” as used herein, refers to, but is not limited to, chemicals, drugs, peptides such as the Senolytic Agent(s) including the Senolytic Peptide(s) or methods that are used prior to, in combination with or following the primary therapy which includes but not limited to surgery in order to enhance or modify the effect of the primary therapy, lower the risk of cancer recurrence and increase patient survival.

“Adjuvant therapy” as used herein, refers to, but not limited to, the therapy or therapies that are used prior to, in combination with; concomitant or concurrent therapy or following the primary therapy in order to enhance or modify the effect of the primary therapy which includes but not limited to surgery, lower the risk of cancer recurrence and increase patient survival. The adjuvant therapy includes, but is not limited to, chemotherapy, radiation therapy, hormone therapy, targeted therapy, biological therapy or other novel therapies that will be recognized by those skilled in the art.

“Administration” or “administering” “administer(ed)” as used herein, refers to a method of giving a dosage of a compound or composition to subject, such as a vertebrate or invertebrate, including a mammal, a bird, a fish, or an amphibian, via a suitable mode of administration, for example, intra-respiratory, topical, oral, intravenous, intraperitoneal, intramuscular, buccal, rectal, sublingual and/or intrathecal. In various embodiments as will be disclosed herein, the preferred mode of administration can vary depending on various factors, such as the components being administered, the tissue site being targeted (e.g., a tissue in which the disease or disorder resides, is present, or is manifested which may be a tumor), the particular disease or disorder involved, and the severity of the disease or disorder.

“Autoimmune disease(s) or disorder(s),” as used herein, refers to autoimmune diseases or disorders such, but not limited to, osteoporosis, psoriasis, oral mucositis, rheumatoid arthritis, inflammatory bowel disease, eczema, kyphosis, herniated intervertebral disc, and the pulmonary diseases, COPD, and idiopathic pulmonary fibrosis.

“Biological sample” or “biopsy sample,” as used herein, refers to a biological sample which is obtained from a subject by invasive, non-invasive or minimally invasive methods, for example, a blood sample, a serum sample, a plasma sample, a biopsy specimen, body fluids (for example, lung lavage, ascites, mucosal washings, synovial fluid, vitreous fluid, or spinal fluid), bone marrow, lymph nodes, tissue explant, skin tissue sample, vaginal tissue, organ culture, or any other tissue or cell preparation from a subject.

“Cancer,” as used herein, refers to, but is not limited to, cancers which are solid tumors or liquid tumors. Solid tumors may include, for example, prostate cancer, testicular cancer, breast cancer, brain cancer (including glioblastoma), pancreatic cancer, colon cancer, colorectal cancer, thyroid cancer, stomach cancer, lung cancer, ovarian cancer, Kaposi's sarcoma, skin cancer (including squamous cell skin cancer), renal cancer, head and neck cancers, throat cancer, squamous carcinomas (e.g., that form on the moist mucosal linings of the nose, mouth, throat, etc. such as laryngeal and hypopharyngeal cancers), bladder cancer, osteosarcoma (bone cancer), cervical cancer, endometrial cancer, esophageal cancer, liver cancer, hepatocellular carcinoma, and kidney cancer and further including the metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells. Liquid tumors may include, for example, cancers occurring in blood, bone marrow, and lymph nodes and include generally, leukemias (myeloid and lymphocytic) including acute lymphoblastic leukemia (ALL), acute myeloid leukemia (AML), chronic lymphocytic leukemia (CLL), chronic myelogenous leukemia (CML), and hairy cell leukemia lymphomas (e.g., Hodgkin lymphoma), and melanoma, including multiple myeloma).

“Cancer stem cells” (CSC) as used herein, refers to, but is not limited to, cancer cells that are tumorigenic or tumor initiating. CSCs possess properties of normal stem cells such as self-renewal to generate more CSCs and differentiation into different cell types to generate non-CSCs and increase tumor burden. CSCs may be a distinct population of cells within a cancer that result in cancer progression, relapse, and/or metastasis. CSCs may exhibit stem cell plasticity, where the cell-state transitions between a non-CSC and a CSC.

“Cardiovascular disease(s)” as used herein, refers to, but is not limited to, angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance, and stroke.

“Chemotherapeutic agent(s)” as used herein, refers to a drug used to treat cancer. Examples of chemotherapeutic agents include but is not limited to Tamoxifen, Femara, Herceptin, Letrozole, Taxol, Soltamox, Epirubicin, Trastuzumab, Leuprolide, Paclitaxel, Ellence, Pharmorubicin PFS, Neratinib, Nerlynx, Ogivri, Opdivo, Intron A, Sylatron, Yervoy, Interferon alfa-2b, Nivolumab, Proleukin, Pembrolizumab, Dacarbazine, Keytruda, Aldesleukin, Lmlygic, Ipilimumab, DTIC-Dome, Temozolomide, Peginterferon alfa-2b, Talimogene laherparepvec, Mekinist, Tafinlar, Zelboraf, Trametinib, Dabrafenib, Vemurafenib, Cotellic, Isotretinoin, Peglntron, Braftovi, Mektovi, Cobimetinib, Binimetinib, Encorafenib, Xeloda, Oxaliplatin, Avastin, Fluorouracil, Leucovorin, Capecitabine, Irinotecan, Stivarga, Bevacizumab, Erbitux, Camptosar, Cetuximab, Eloxatin, Vectibix, Zaltrap, Betaseron, Fusilev, Lonsurf, Methotrexate, Panitumumab, Wellcovorin, Regorafenib, Mvasi, Cyramza, Interferon beta-1b, Levoleucovorin, Ramucirumab, Tipiracil/Trifluridine, Ziv-aflibercept, Khapzory, Temodar, Temozolomide, Matulane, BiCNU, Gliadel, Carmustine, Hydroxyurea, Procarbazine, Gleevec, Mercaptopurine, Sprycel, Adriamycin, Dasatinib, Trexall, Iclusig, Blincyto, Pegaspargase, Doxorubicin, Imatinib, Oncaspar, Purinethol, Blinatumomab, Erwinaze, Ponatinib, Vincristine liposome, Asparaginase Erwinia chrysanthemi, Marqibo, Vumon, Xatmep, Arranon, Besponsa, Clolar, Kymriah, Clofarabine, Nelarabine, Purixan, Teniposide, Inotuzumab ozogamicin, Tisagenlecleucel, Prednisone, Dexamethasone, Sprycel, Cytarabine, Dexamethasone Intensol, Triamcinolone, Idarubicin, Clinacort, Idamycin, Fludarabine, Kenalog-40, Dexpak Taperpak, De-Sone LA, Idamycin PFS, Bicalutamide, Casodex, Zytiga, Lupron Depot, Xtandi, Zoladex, Eligard, Firmagon, Abiraterone, Provenge, Taxotere, Enzalutamide, Goserelin, Trelstar, Degarelix, Estrace, Estradiol, Delestrogen, Docetaxel, Estradiol Patch, Flutamide, Triptorelin, Jevtana, Nilandron, Zofigo, Zoladex 3-Month, Cabazitaxel, Premarin, Conjugated estrogens, Cyclophosphamide, Menest, Nilutamide, Novantrone, Sipuleucel-T, Trelstar Depot, Trelstar LA, Vantas, Apalutamide, Emcyt, Erleada, Eulexin, Histrelin, Mitoxantrone, Radium 223 dichloride, Viadur, Yonsa, Esterified estrogens, Estramustine, Premarin intravenous, Supprelin LA, Tarceva, Alimta, Iressa, Erlotinib, Cisplatin, Gemzar, Abraxane, Tagrisso, Xalkori, Navelbine, Pemetrexed, Gefitinib, Crizotinib, Gilotrif, Venorelbine, Platinol, Tecentriq, Afatinib, Gemcitabine, Ceritinib, Platinol-AQ, Photofrin, Alecensa, Alectinib, Paclitaxel protein-bound, Porfimer, Zykadia, Atezolizumab, Durvalumab, Imfinzi, Necitumumab, Osimertinib, Alunbrig, Brigatinib, Dacomitinib, Infugem, Lorbrena, Lorlatinib, Portrazza, Vizimpro, Etoposide, Topotecan, Hycamtin, VePesid, Toposar, Etopophos, Armour Thyroid, Nexavar, Nature-Throid, Thyrogen, Caprelsa, Cometriq, Thyroid desiccated, Sodium iodide-i-131, Sorafenib, Lenvima, Lenvatinib, Lodotope, Cabozantinib, Westhroid, Thyrotropin alpha, Vandetanib, Hicon, NP Thyroid, WP Thyroid, i3odine Max, Carboplatin, Doxil, Doxorubicin liposomal, Alkeran, Paraplatin, Lynparza, Olaparib, Zejula, Cosmegen, Melphalan, Rubraca, Dactinomycin, Niraparib, Tepadina, Rucaparib, Thiotepa, Afinitor, Sutent, Everolimus, Pancreatin, Zanosar, Mutamycin, Onivyde, Sunitinib, Streptozocin, Mitomycin, Irinotecan liposomal, Bleomycin, Velban, Ifex, Ifosfamide, Vinblastine, Bleo 15k, lomustine, CeeNU, Gleostine, Panretin, Alitretinoin, Votrient, Inlyta, Pazopanib, Torisel, Axitinib, Medroxyprogesterone, Cabometyx, Temsirolimus, 5-fluorouracil, Cemiplimab, Libtayo, Caboplatin, Megestrol, Provera, Anastrozole, Depo-Provera, Depo-subQ provera 104, Depo-Provera Contraceptive, Profimer and their derivatives.

“Chemotherapy” as used herein, refers to administering Chemotherapeutic agent.

“Circulating tumor cells (CTCs)” as used herein, refers to, but is not limited to, epithelial cells found in circulation with either intact viable nuclei and with fragmented, apoptotic nuclei. CTCs have extravasated from the primary tumor and will ultimately intravasate into a distant tissue forming a metastasis. CTCs can be marked by EPCAM, CK, or any other epithelial or mesenchymal specific marker. CTCs can be marked by MUC4 or epithelial-cancer specific marker. CTCs can be isolated from blood using antibody based separation, magnetic separation, size based separation, magnetic levitation or other means. CTCs can be either negative for blood cell specific markers such as CD45, or positive for blood specific markers, also known as circulating hybrid cells (CHCs).

“Dormant cells”, as used herein, refers to, but is not limited to, cancer cells that are non-dividing and arrested in any state of the cell cycle. Dormant cells can be present in the primary tumor, in micrometastases, in lymph nodes, in distant tissues, or in residual disease after treatment. Dormant cells can reside within the body for any length of time and can be re-activated at any time. Dormant cells can also include cells just after extravasation to a metastatic site such as lung, liver, brain, bone, lymph node and all other tissues.

“Inflammatory or autoimmune disease(s) or disorder(s),” as used herein, refers to, but is not limited to, inflammatory diseases or disorders, such as by way of non-limiting example, osteoarthritis, or autoimmune diseases or disorders, such as by way of non-limiting example, osteoporosis, psoriasis, oral mucositis, rheumatoid arthritis, inflammatory bowel disease, eczema, kyphosis, herniated intervertebral disc, and the pulmonary diseases, COPD and idiopathic pulmonary fibrosis.

“Neoadjuvant therapy” as used herein, refers to, but is not limited to, the treatment given as a first step to shrink a tumor before the main treatment, which is usually surgery, is given. Examples of neoadjuvant therapy include chemotherapy, radiation therapy, hormone therapy or other novel therapies that will be recognized by those skilled in the art.

“Peptidomimetics,” as used herein, refers to certain chemical compounds mimicking a natural peptide with the ability to interact with the target and exert the same biological effect. Peptidomimetics may be often implemented to overcome problems associated with the intrinsic properties of natural peptides such as stability against proteolytic degradation. Peptide bond surrogates and β-turn dipeptide mimetics are included among examples of peptidomimetics.

“Primary therapy” “Primary Treatment” as used herein, refers to but is not limited to the first treatment given for a disease which includes standard set of treatments, such as surgery followed by chemotherapy and radiation. When used by itself, primary therapy is the one accepted as the best treatment. If it doesn't cure the disease or it causes severe side effects, other treatment may be added in following therapies. Examples of the primary therapy includes but is not limited to, surgical therapy, radiotherapy, chemotherapy, immunotherapy, targeted therapy, hormone therapy, biological therapy or other novel therapies that will be recognized by those skilled in the art.

“Pulmonary disease(s) and disorder(s),” as used herein, refers to, but is not limited to, idiopathic pulmonary fibrosis (IPF), chronic obstructive pulmonary disease (COPD), asthma, cystic fibrosis, bronchiectasis, and emphysema.

“Senescence associated dermatological disease(s) and disorder(s)” as used herein, refers to, but is not limited to, psoriasis, vitiligo, and eczema, which are also inflammatory diseases and are discussed in greater detail. Other dermatological diseases and disorders that may be associated with senescence include rhytides (wrinkles due to aging); pruritis (linked to diabetes and aging); dysesthesia (chemotherapy side effect that is linked to diabetes and multiple sclerosis); psoriasis (as noted) and other papulosquamous disorders, for example, erythroderma, lichen planus, and lichenoid dermatosis; atopic dermatitis (a form of eczema and associated with inflammation); eczematous eruptions (often observed in aging subjects and linked to side effects of certain drugs). Other dermatological diseases and disorders associated with senescence include cutaneous lymphomas, eosinophilic dermatosis (linked to certain kinds of hematologic cancers); reactive neutrophilic dermatosis; pemphigus, cutaneous lupus, pemphigoid and other immunobullous dermatosis fibrohistiocytic proliferations of skin.

“Senescence related diseases or disorders,” as used herein, refers to, but is not limited to, any of the following diseases or disorders a senescence-related disease or disorder, an inflammatory disease or disorder, an autoimmune disease or disorder, a cardiovascular disease or disorder, a pulmonary disease or disorder, an ophthalmic disease or disorder, a metabolic disease or disorder, a neurological disease or disorder, a senescence-associated dermatological disease or disorder, a nephrological disease or disorder, renal dysfunction, kyphosis, herniated intervertebral disc, frailty, hair loss, hearing loss, muscle fatigue, a gradual loss of function, or degeneration that occurs at the molecular, cellular, tissue, and organismal levels.

“Senolytic Agent(s),” as used herein, refers to, but is not limited to: Dasatinib, Quercetin, Navitoclax, Piperlongumin, Fisetin, BCL-XL inhibitors A1331852 and A1155463, all FoxO4-related peptides and Foxo4-CR3 domain inhibiting peptides including Senolytic Peptide(s).

“Senolytics,” as used herein, refers to one or more of the Senolytic Peptides or Senolytic Agents.

“Senolytic Peptide(s),” as used herein, refers to the cell penetrating peptide sequences disclosed herein, including the peptides shown in the SEQUENCE LISTING and derivatives thereof.

“Senolytic Therapy” as used herein refers to administration of therapeutic regimes in one or more treatments cycles for prevention or treatment of senescence-associated diseases and disorders and for prevention, cure, inhibition or retarding the progression of cancer and/or metastasis as wherein Senolytic Agents are used alone or in combination or as an adjuvant, or for prevention, inhibition or retarding the progression of a Senescent Related Diseases or Disorders specifically including Cancer and or metastasis of a Cancer.

Treatment Regime(s) as used herein refers to administration of Senolytic Agents in therapeutically Effective Dose in composition and in delivery methods and treatment cycles and regimes disclosed in the subject matter of this patent. Treatment Regimes definition includes but not limited to regimes described in Schedule 1 and Impulse, Shock and Sustained regimes variations thereof.

“Therapeutically Effective Dose”, as used herein, refers to a dose which cause a therapeutic effect to some extent, some effect with respect to one or more of the symptoms of the disease, and includes curing a disease. “Curing” means that the symptoms of active disease are eliminated. However, certain long-term or permanent effects of the disease may exist even after a cure is obtained (such as where extensive tissue damage is present). The therapeutically Effective Dose is typically one which is sufficient to achieve the desired effect and may vary according to the nature and severity of the disease condition, and the potency of the particular Senolytic Peptide administered, its purity, and its composition (e.g. 90%, or 95% or 98% etc.). In some embodiments, different doses may be employed for embodiments where the Senolytic Peptide(s) is administered for preventive use rather than for treatment of an active disease. therapeutically Effective Dose definition shall include the dosage for the Senolytic Agents as disclosed in Schedule 1.

“Subject,” as used herein, refers to a target of a treatment or therapy, including but not limited to, a human or a non-human mammal, a dog, a cat, a mouse, a rat, a cow, a sheep, a pig, a goat, a non-human primate or a bird, for example, a chicken, as well as any other vertebrate or invertebrate.

“Treat,” “treatment,” or “treating,” as used herein, refers to administration of a composition for therapeutic purposes and includes the Treatment Regimes as disclosed in the subject matter of the patent.

Disclosed herein is a successful model for the interaction network between the domains of FoxO4 and p53 at atomic resolution, by which the selective inhibition of the action of FoxO4 on p53 was achieved.

Both FoxO4 and p53 have multiple domains with distinct functions. Prior art investigations as to possible interactions of FoxO4 with p53 implied that FoxO4 can interact with p53 through multiple domains. Not intending to be bound by theory, these potential interactions between FoxO4 and p53 may explain the cause of the restrain elicited by FoxO4 on the apoptotic function of p53. Hence in the rational design, the possible complexes of FoxO4 and p53 was elucidated at the atomic resolution.

The FoxO family of proteins express similar domain compositions such that they contain DNA binding domains entitled as the Forkhead (FH) domain and C-terminal domains entitled as CR1, CR2 and CR3 for transactivation. The FH and CR3 domains interact with each other and the binding surface of these interactions have been resolved in an NMR based study using FoxO3. FH includes basic (positively charged) and hydrophobic amino acids (R153, R154, W157 and G158) that contribute to this intramolecular interaction, while a central portion of CR3 that includes acidic (negatively charged) and hydrophobic amino acids (the amino acids from D623 to M633) interacts with the FH.

This NMR study also shed light on the action of FoxO3 on p53. Essentially, in vitro pull down assays were used to identify the critical domain(s) of both p53 and FoxO3 for their intermolecular interactions. According to these results, the DNA binding domain of p53 (p53DBD) was found to be necessary for binding to the FoxO3, while the C-terminal portion of the FoxO3 that includes the CR3 domain was revealed to be the most critical domain for p53 interaction. Further experiments showed that addition of the p53DBD disrupted the intramolecular interaction between the FH and the CR3 domains of FoxO3, in that the binding affinities of both of the FH-CR3 and p53DBD-CR3 complexes were comparable with each other. As resolved by NMR shift experiments, the binding interface of the CR3 domain of FoxO3 has been overlapped in both of the complexes formed by the FH domain of FoxO3 and the p53DBD. This particular finding implied a competition of the FH domain of FoxO3 with the p53DBD to bind to the same surface on the CR3 domain of FoxO3. Sequestration of the CR3 domain, particularly from the surface that it binds to p53DBD will circumvent its interaction with the p53DBD, liberating the p53DBD and its transcriptional activity to initiate apoptosis.

The FoxO proteins are very similar proteins that share high sequence similarity and domain composition. This similarity in the protein sequence was further observed at the functional level by in vivo experiments in mice. The resulting functional redundancy will suggest the validity of the findings on the FoxO3 for FoxO4. Hence, in connection to the insights obtained from the NMR findings, blocking of the CR3 domain of FoxO4 has the potential to inhibit the interaction between FoxO4 and p53 and thus liberate p53 in senescent cells.

Interference in the p53 structure or pathway may bear harmful consequences to cells and thus should be avoided by any means. Nevertheless the structural findings suggested that the CR3 domain of FoxO4 binds to and possibly inhibits the function of the p53DBD. Blocking of the CR3 domain will inhibit “the inhibition of the p53DBD” and will promote the function of p53DBD. Experiments conversely showed that promoting the inhibition of p53 by an oncoprotein, named gankryin, enhanced tumor growth. Gankyrin acts as a promoter of MDM2 which is the well-known inhibitor of p53. Thus interference with the CR3 domain of FoxO4, without any direct interference with the p53 protein, will have the potential to remove the said restrain on the p53 mediated apoptosis by FoxO4, eradicating the senescence.

Senolytic Cell Penetrating Peptides for the Treatment of Senescence Associated Disorders

Generally, CPPs are between 5 and 40 amino acids in length, and display great sequence variety. Many CPPs are highly positively charged, and studies have shown that the number of L-arginine residues is critical for transduction activity of cationic CPPs. In some CPPs, cationic regions alternate with hydrophobic regions, giving rise to an amphiphilic often α-helical structure. The mechanism of CPP uptake is not universal and depends on the type of CPP, its cargo and peptide concentration. Macropinocytosis, receptor-mediated endocytosis, and direct membrane penetration have all been described to play a role for CPP uptake. In general, CPPs are either synthesized and then coupled by various means to their cargo or manufactured as recombinant fusion proteins. Intense interest in this technology has led to rapid progress, and clinical trials using CPPs are ongoing. Several aspects, such as unfavorable pharmacokinetics, few targeting strategies and overall low efficiency still present challenges to the field. Peptides generally have a short half-life time due to proteolysis and renal clearance, and non-specific binding of CPPs can lead to a dramatic reduction in delivery to target cells. As an efficient delivery vehicle CPPs could address some of these shortcomings and represent a new platform for therapeutic peptide or protein delivery. Some progress has been made through the use of unnatural amino acids, PEGylation and other peptide modifications, but systemic delivery remains inefficient, and oral bioavailability is lacking.

FoxO4 is a well-established target for selectively clearing senescent cells. The Associated Disclosed Material 1 discusses the structural mechanism behind targeting the negatively charged domain (CR3) of FoxO4 for treatment of senescence-associated diseases and disorders. This invention leverages the particular Foxo4 inhibition mechanism described in said Associated Disclosed Material 1 and employs it to identify novel peptides. According the Disclosed Material 1, peptides from the CPP database (http://crdd.osdd.net/raghava/cppsite/) along with a positively charged peptide (ID #1 in the Table 1) have been screened for targeting the CR3 domain of the FoxO4. The selected CPPs in Table 1 were discovered to bind to FoxO4 strongly in silico, thus said selected CPPs are suitable to target senescent cells to apoptosis wherein said treatment stimulates overall rejuvenation in a safe manner.

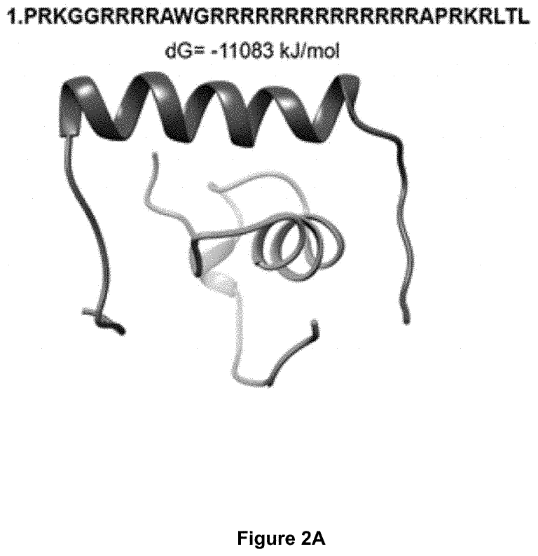

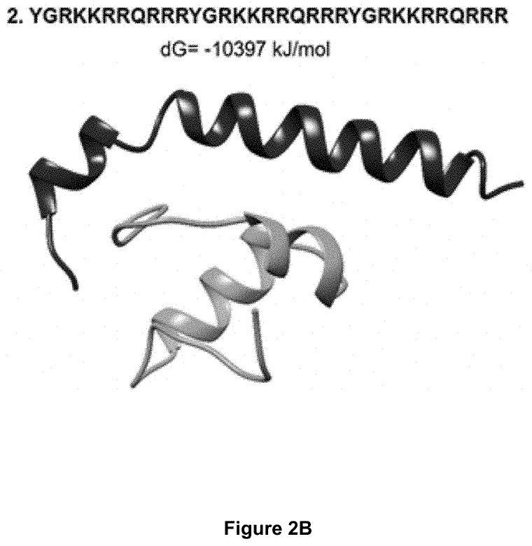

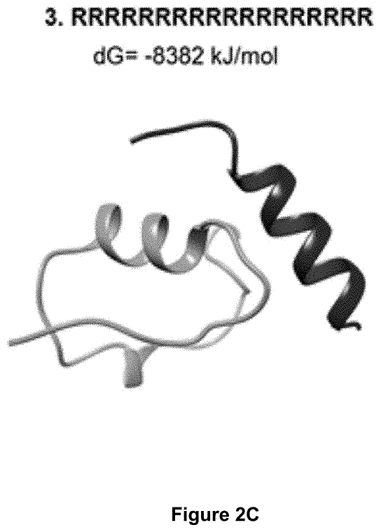

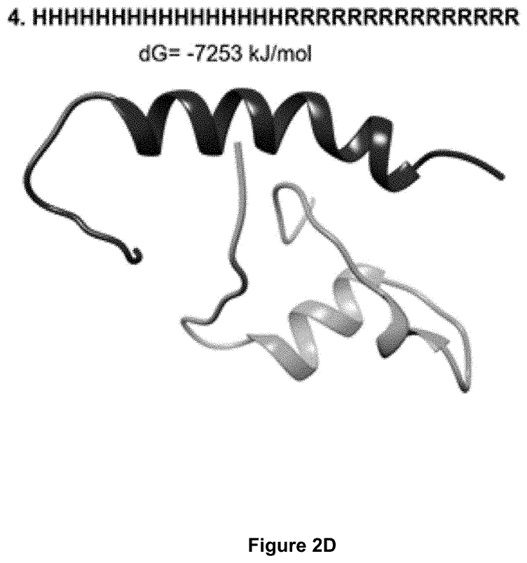

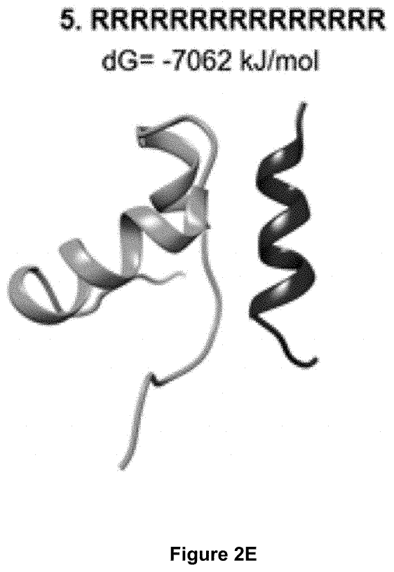

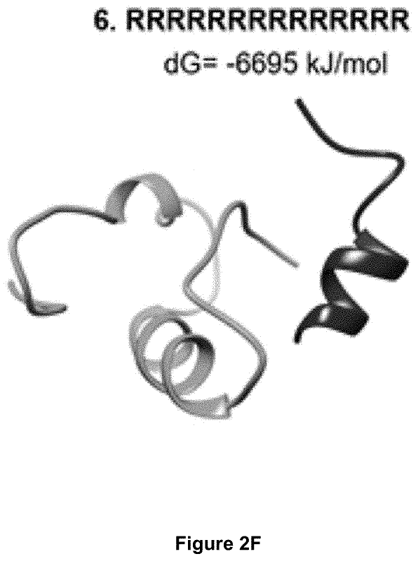

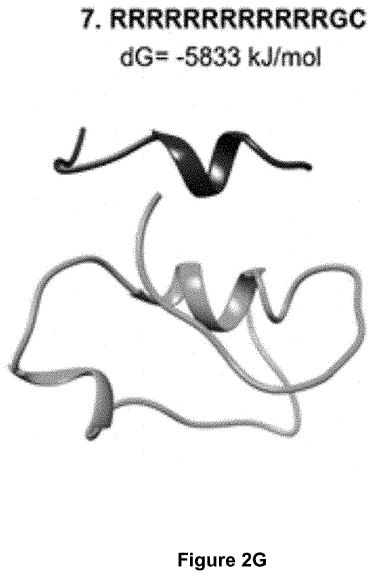

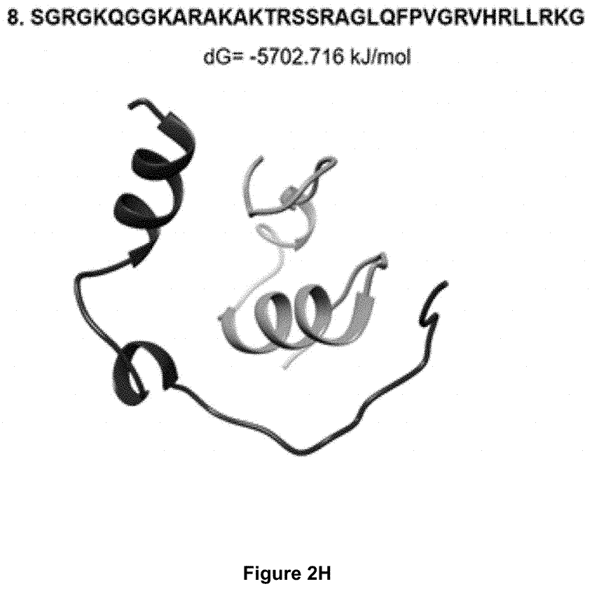

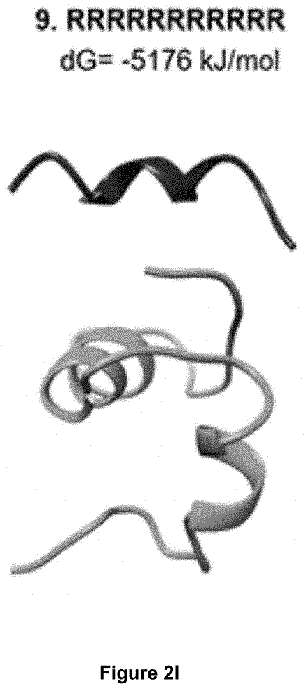

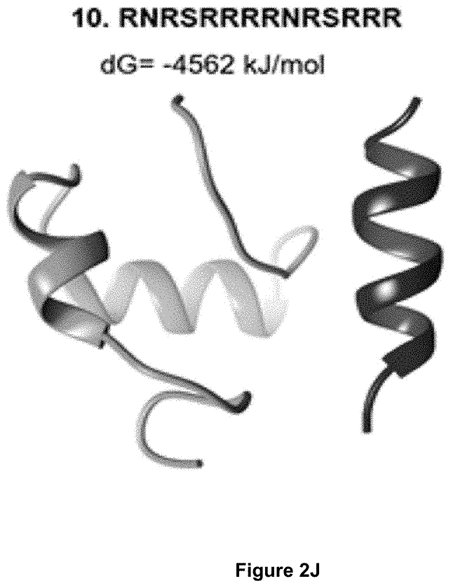

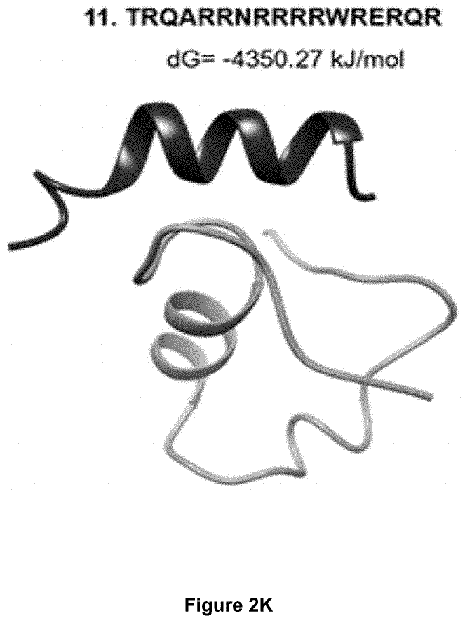

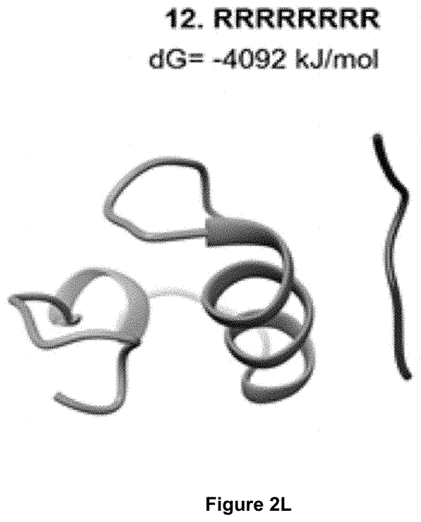

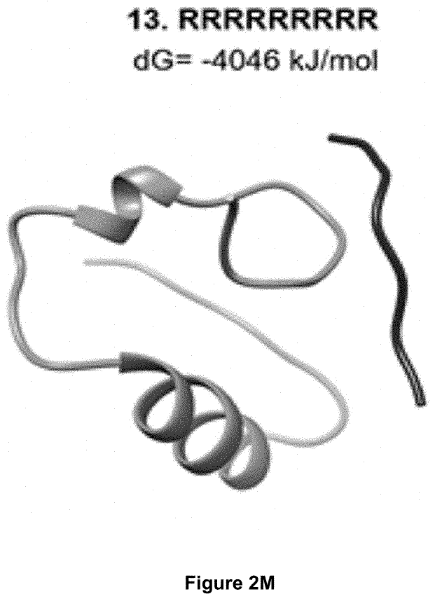

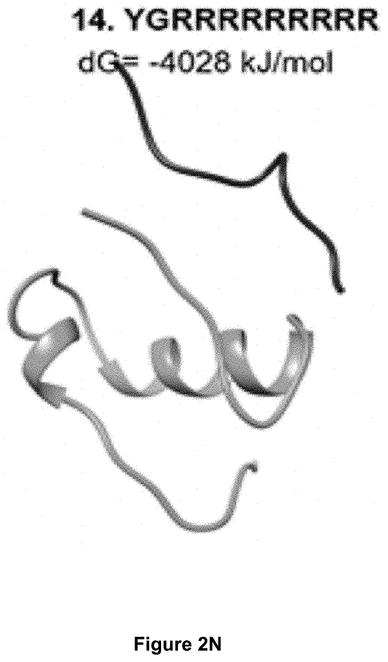

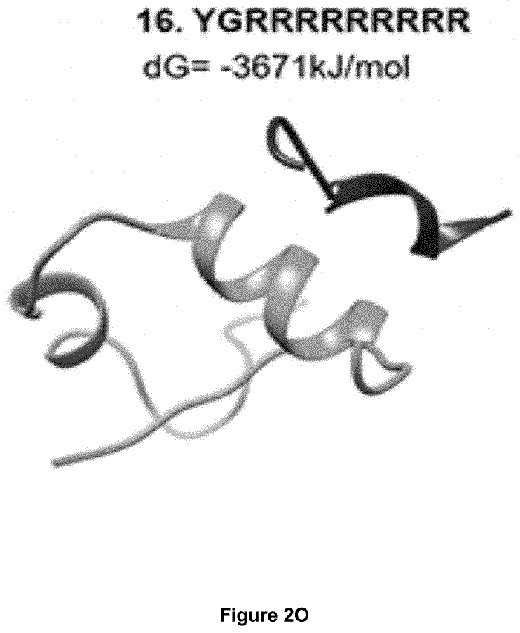

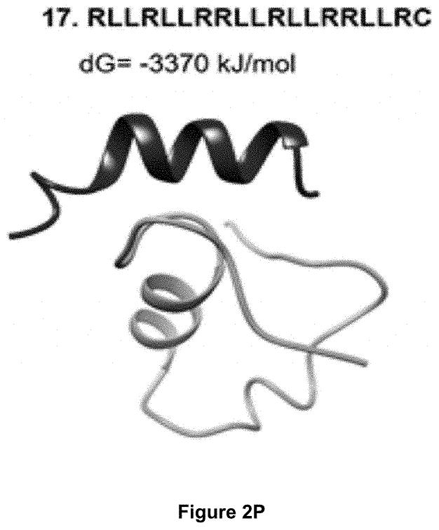

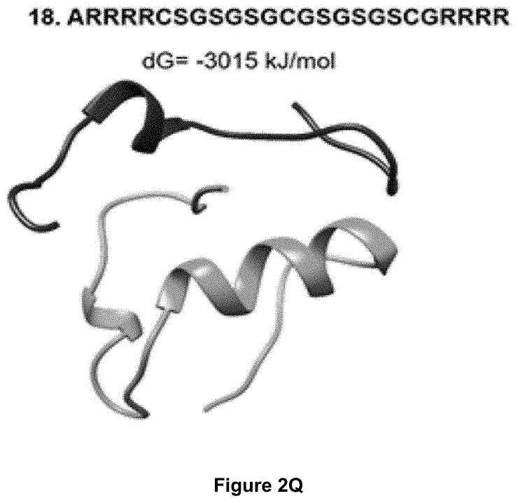

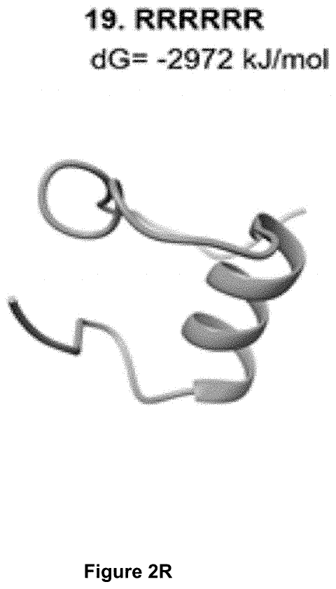

The target described in Associated Disclosed Material 1, CR3 domain of FoxO4 has been analysed in molecular dynamics simulations and 20 different conformations were obtained to be tested in structure-based virtual screening. The CPP library (http://crdd.osdd.net/raghava/cppsite/) (1200 CPPs excluding the toxic peptides) was screened by LeDock (http://www.lephar.com/software.htm) and using the CR3 domain conformations as the target. Protein-Ligand Interaction Profiler (Plip) tool (http://plip.biotec.tu-dresden.de/plip-web/plip/index) was used to analyze the intermolecular interactions of all 20 conformations. Plip results were given in Table 1. According to this analysis, the conformations that gave the best Plip value were selected for dynamical analysis. The selected complex structures were analysed by molecular dynamics simulations which lasted for 40 ns. The last 1 ns of these simulations were used to predict the binding free energy of the CPP to the CR3 by the MM-PBSA method. Overall, both MM-PBSA calculations and docking simulations led us to conclude that 20 different CPPs carry high potential for targeting FoxO4 to treat senescence-associated diseases and disorders as shown in Table 1. FIGS. 2A-2R show CPPs listed in the Table 1 in complex with CR3 domain of FoxO4. Our extensive in silico analysis showed that CPPs of Table 1 are able to bind to the CR3 domain of FoxO4, based on the net positive charge on these peptides. All of the CPPs listed in the Table 1 are rich in arginine amino acids. Particularly, 12 of them with the IDs of 1, 3, 4, 5, 6, 7, 9, 12, 13, 14, 15, 16 and 19 in the Table 1 have at least one hexa polyarginine repeat in their sequence. Despite the number and the length of the polyarginine repeats have differed in the CPPs in Table 1, arginine rich CPPs show affinity towards the CR3 domain of FoxO4. Further novel derivatives of CPPs of Table 1 containing mutations at most 30% of the native full-length CPP sequence also interfere with CR3 domain of FoxO4 thereby interfering with SCAP. Furthermore, the novel peptides that are formed by combining or shuffling of any part of one or more CPP sequences that is at least 6 amino acid in length shall carry the CR3 domain binding affinity thereby interfering with SCAP. From hereon the senolytic CPPs definition herein this application include those listed in Table 1 and their novel derivatives formed by mutagenesis or insertion/deletion of any parts of the CPP sequences that are at least 6 amino acid in length.

There is strong evidence proposing FoxO4 (not other FoxO members) as the pivot molecule in mediating the cells' fate to apoptosis or senescence. For instance, FoxO4 expression is significantly increased upon senescence compared with those of FoxO1 (not detected) and FoxO3 (not significant) (Baar, 2017, Cell). Thus, other FoxOs (FoxO1, 3a and 6) as a crucial component of senescence pathways. Still due the well studied functional redundancy in FoxO family members, the CPPs that are identified to target FoxO4 carry the potential to target other FoxOs as well. Targeting all FoxOs may bear unwanted side effects, since FoxOs can act as tumor suppressor proteins in normal cells. From this perspective, the binding affinity of the selected CPPs to FoxO3 were also analysed. The results for the peptides with the IDs of 1, 2, 3 of CPPs of Table 1 were given in Table 2. All three of peptides listed in column 1 showing the highest binding affinity to FoxO4, showed less binding affinity to FoxO3, confirming the FoxO4 selectivity of these peptides.

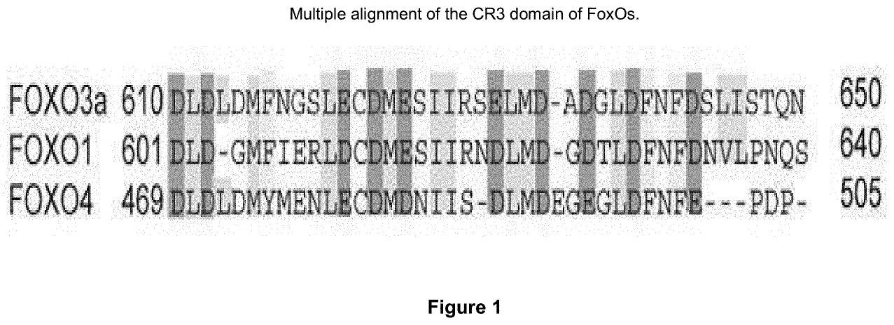

Furthermore, according to the multiple alignment of the CR3 domain of FoxO proteins in human showed that FoxO4 has a high negative charge on the CR3 domain. The invented CPPs are identifited to interact with the FoxO4 CR3 through the negatively charged residues that are specific to FoxO4 but not to FoxO3 and 1 (FIG. 1). Thus, the invented CPPs are more potent to inhibit the CR3 domain of FoxO4 than those of other FoxOs. In some embodiments, CPPs that are generated by repetition or shuffling of CPP domains that are rich in arginine, lysine or histidine are effective to target the negatively charged CR3 domain of Foxo4.

Peptides with D-amino acids can mimic L-peptides and modification to D-retroinverso (DRI) forms often change the peptide to a more potent form in vitro and in vivo. These DRI peptide forms have also been shown to be therapeutically effective in clinical trials. As will be recognized by those skilled in the art the CPPs described in Table 1 could be expressed by using D-amino acids and/or in a retroreversed sequence. In some specific embodiments, the senolytic CPPs are expressed by using D-amino acids and/or in a retroreversed sequence. In one embodiment, the senolytic peptide is the unique artificial peptide version of senolytic CPPs which comprises all D-amino acids or a mixture of D- and L-amino acids. As will be recognized by those skilled in the art, peptides with single or multiple L- and/or D-amino acid additions to the peptide sequence will overcome the degradation signals found in the N-terminus, resulting in a longer half life of the peptide. In one embodiment, senolytic CPPs are expressed with single and/or multiple D-amino acid insertion to N-terminus. In one embodiment, senolytic CPPs are expressed with single and/or multiple D-amino acid insertions. In one embodiment, senolytic CPPs are expressed with single and/or multiple L-amino acid insertions.

As will be recognized by those skilled in the art, the senolytic CPPs and the disclosed method described in the present application can be modified to optimize their half-life. In some embodiments these modifications of the senolytic CPPs are N-Terminal Modifications (acetylation, biotinylation, dansyl and 2,4-Dinitrophenylation, fluorescent dye labelling, 7-methoxycoumarin acetic acid (Mca) modification, palmitic acid modification), Internal Modifications (cyclization by disulfide bonds, cysteine carbamidomethylation (CAM), isotope labelling, phosphorylation, spacer addition, PEGylation, amino hexanoic acid modification), C-Terminal Modifications (amidation). In preferred embodiments of the said senolytic CPPs are further modified by any of peptide modification approaches to optimize their stability.

Peptidomimetics are chemical compounds mimicking a natural peptide with the ability to interact with the target and exert the same biological effect. Peptidomimetics are often recruited to overcome problems associated with the intrinsic properties of natural peptides such as stability against proteolytic degradation. Peptide bond surrogates and β-turn dipeptide mimetics among examples of peptidomimetics. As will be recognized by those skilled in the art the invented peptide(s) could be expressed as a peptidomimetics. In other embodiments, CPPs are expressed as a peptidomimetics.

Cyclic peptides has been shown to be more effective than their linear counterparts due to the conformational rigidity. Moreover cyclic structure becomes more resistant to proteolytic cleavage by exopeptidases due to the termini and by endopeptidases as their structure is more rigid than linear peptides. As will be recognized by those skilled in the art the invented peptide(s) could be expressed in unnatural amino acids and in cyclic form. In some specific embodiments of the senolytic CPPs are expressed by unnatural amino acids and/or in cyclic form.

Unnatural or unusual amino acids are not naturally encoded amino acids. These are non-proteinogenic amino acids which can occur naturally in plant or bacteria post-translationally or are chemically synthesized as pharmacological motifs. Unnatural amino acid are widely used in combinatorial libraries or as chiral building blocks. These modified amino acids often contribute to special biological activity, and are often incorporated into therapeutic peptidomimetic ligands to enhance peptide pharmacological activity and potency. Unusual or unnatural amino acid modified peptides can improve selectivity, receptor binding, modify activity (agonists and antagonists), increase in vivo half-life, enhance transport across cell membranes. As will be recognized by those skilled in the art, the invented CPPs are expressed by a wide range of unnatural, non-standard amino acid peptide modifications. In some embodiments, modifications to CPPs include incorporation of unnatural/unusual amino acids into the sequence using D-amino acids, L-amino acids, N-methyl amino acids, homo amino acids, alpha-methyl amino acids, beta (homo) amino acids, gamma amino acids, helix/turn stabilizing compound or backbone modification peptoides. In other embodiments, CPP sequence(s) are modified by insertion of natural occurring unusual amino acids such as citrulline (Cit), hydroxyproline (Hyp), beta-alanine, ornithine (Orn), norleucine (Nle), 3-nitrotyrosine, pyroglutamic acid (Pyr), nitroarginine, Alanine Modified Amino Acid Analogs, Alicyclic Amino Acids Analogs, Aromatic Amino Acids Analogs, Aspartic Acid Analogs, Beta-Amino Acids Analogs, Cysteine Amino Acid Analogs, DAB (2,4-Diaminobutyric Acid) Analogs, DAP (2,3-Diaminopropionic Acid) Analogs, Depsipeptides, Glutamic Acid Amino Acid Analogs, Glutamine Amino Acid Analogs, Glycine Amino Acid Analogs, Heterocyclic Amino Acid Analogs, Histidine Amino Acid Analogs, Homo-Amino Acid Analogs, Isoleucine Amino Acid Analogs, Leucine Amino Acid Analogs, Linear Core Amino Acid Analogs, Lysine Amino Acid Analogs, Methionine Amino Acid Analogs, N-Methyl Amino Acids Analogs, Norleucine Amino Acid Analogs, Norvaline Amino Acid Analogs, Ornithine Amino Acid Analogs, Statine Amino Acid Analogs, Penicillamine Amino Acid Analogs, PEGylated Amino Acids, Phenylalanine Amino Acid Analogs, Phenylglycine Amino Acid Analogs, Proline Amino Acid Analogs, Pyroglutamine Amino Acid Analogs, Serine Amino Acid Analogs, Threonine Amino Acid Analogs, Tryptophan Amino Acid Analogs, Tyrosine Amino Acid Analogs, Valine Amino Acid Analogs, Post-Translational Modification (PTM) Modified Amino Acids, Deiminated Arginines (Citrulline), DL-Citrulline, L-Citrulline, Methylated Aminod Acids, Lysine(Me), Lysine(Me2), Lysine(Me3), Arginine(Me), Arginine(Me)2 asymmetrical, Arginine(Me)2 symmetrical, Phosphorylated Amino Acids, Phosphoserine, phosphothreonine, phosphotyrosine.

In other embodiments, polynucleotides that are complementary to at least a portion of a nucleotide sequence encoding the designed peptides (e.g., a short interfering nucleic acid, an antisense polynucleotide, or a peptide nucleic acid). These polynucleotides that specifically bind to or hybridize to nucleic acid molecules may be prepared using the peptide sequences available in the art to alter gene and/or protein expression. As will be recognized by those skilled in the art the CPPs could be expressed as a product of polynucleotides. In a particular embodiment, the polynucleotides may be delivered by a recombinant vector in which the polynucleotide of interest has been incorporated. In other embodiments, the recombinant vector may be a viral, a non-viral vector. In another embodiment, the recombinant vector may be a recombinant expression vector into which a polynucleotide sequence that encodes the designed peptides. In some embodiments the recombinant vector or the recombinant expression vector is a viral recombinant vector or a viral recombinant expression vector. In other embodiments the recombinant vector or the recombinant expression vector is a non-viral recombinant vector or a non-viral recombinant expression vector. As will be recognized by those skilled in the art the senolytic CPPs could be expressed from a vector.

Method of Administration of the Senolytic Peptides

The following are certain embodiments described in greater detail for selectively killing senescent cells in a subject who has a senescence associated disease or disorder with Senolytic Peptides. For treatment of senescence-associated diseases and disorder, Senolytic Peptides shall be administered via intravascular or intramuscular by three different shock dose regimes namely Impulse Regime, Sustained Shock Regime, and Gentle Shock Regime.

This patent discloses well-defined therapies for treating senescence-associated diseases and disorders by administering the Senolytic Peptides. For said therapies the Senolytic Peptides are administered by following three different shock dose regimes namely Impulse Regime, Sustained Shock Regime, and Gentle Shock Regime. Said dose regime is defined as the amount of senolytic agent that is expressed in terms of the quantity and the frequency. Said amount shall be called the Effective Dose. In one embodiment the Effective Dose may be defined as a therapeutically-effective amount of any of, Senolytic Peptides or in combination that selectively kills senescent cells over non-senescent cells so that a pre-targeted ratio of the senescent cells over non-senescent cell is achieved at the end of each shock regime. Said targeted cell ratio could be any ratio from approximately zero or 20%. The effective dose and the duration of treatment vary in different regimes depending on the patients' state of health and how the patients respond to the treatment which can be monitored by histopathological analysis of the senescent cells throughout the period of the therapy. In some embodiments Effective Dose for the senolytic CCPs is as described in Schedule 1. In other embodiments, Effective Dose for the senolytic iporimidite compounds shall be as described in Schedule 2.

The treatment methods for treating a senescence-associated disease or disorder that further with either one of the Impulse, Sustained, and Gentle shock regimes comprises monitoring the population of the senescent cells on a subject at the beginning and 2 or 4 weeks after the treatment to determine the efficacy of the therapy and to determine whether a second or a third treatment course may be required. Said monitoring of senescent cell population at the beginning of a therapy and a certain period after each treatment course shall be called individualized treatment course. This method comprises first detecting the level of senescent cells in the subject, such as in a particular organ or tissue of the subject. A biological sample may be obtained from the subject, for example, a blood sample, serum or plasma sample, biopsy specimen, body fluids (e.g., lung lavage, ascites, mucosal washings, synovial fluid, vitreous fluid, spinal fluid), bone marrow, lymph nodes, tissue explant, organ culture, or any other tissue or cell preparation from a subject. Those familiar with art will be able to detect the senescent cell by using senescence associated markers such as, senescence-associated β-galactosidase (SA-β-gal), p16INK4a, p21, PAI-1, or any one or more SASP factors. In some embodiments, the therapy based on the Senolytic Peptides shall include said individualized treatment course.

In one embodiment, a method is provided for treating a senescence-associated disease or disorder comprising administering to a subject in need thereof the Effective Dose; wherein said novel senolytic agent(s) is administered intermittently in one or multiple treatment cycles. This regime of administration of the Senolytic Peptides shall be named as Impulse Shock dose regime. In the dose Regime, the Effective Dose is achieved through one or two subsequent administration(s). In some embodiments the full Effective Dose for the Shock Regime may be administered in 1 or 2 or 3 days.

In other embodiments, the Effective Dose is achieved through multiple administrations in the period of 1-3 weeks, wherein the quantity of the senolytic agent for each administration is the same and adjusted to cumulatively achieve the Effective Dose at the end of the treatment. This regime of administration of the Senolytic Peptides shall be named as Sustained Shock dose regime. The Effective Dose administered by the Sustained Shock dose regime is higher than the Effective dose administered by the Impulse Shock dose regime.

In another embodiment, the Effective Dose is achieved through multiple administrations in the period of 3-4 weeks. This regime of administration of the Senolytic Peptides shall be named as the Gentle Shock dose regime. Both the Effective Dose and the quantity of the senolytic agent for a single administration in the Gentle Shock dose regime is lower than those in the Impulse Shock dose regime.

For all of the defined dose regimes, the novel senolytic agent(s) do not have to be continuously present to exert its effect. Brief disruption of pro-survival pathways is adequate to kill senescent cells. The novel senolytic agent(s) are effective when administered intermittently.

It has already been reported that other known senolytic agents such as the tyrosine kinase inhibitor (D) and the flavonoid, quercetin (Q), were shown to induce apoptosis in senescent cells. Intermittent administration of D+Q alleviated frailty, neurological dysfunction, osteoporosis, and vertebral disk degeneration related to loss of glycosaminoglycans on an accelerated aging-like state. Furthermore, in mice with impaired mobility due to radiation of one of their legs 3 months previously, tread-mill endurance improved within 4 days after completing a single course of D+Q. Said improvement persisted for at least 7 months. D+Q has an elimination half-life of a few hours. These outcomes following intermittent or single courses of agents with short elimination half-lives are consistent with the long-lasting type of effect expected from reducing senescent cell abundance, as opposed to what would be expected if D+Q had to be continuously present to suppress or activate cellular processes by occupying a receptor or acting on an enzyme. Similarly FoxO4-p53 interfering peptide, FoxO4-DRI administration was shown to be the effective when applied intermittently.

Thus, intermittent rather than continuous treatment with senolytics may be effective in alleviating senescence-related diseases or disorders, allowing these agents to be administered during periods of good health and potentially decreasing risk of side-effects. In the shock dose regimes, there will be an optional follow-up treatment wherein the Effective Dose can be increased or decreased. In between the initial treatment and follow-up treatment there will be a non-treatment interval, wherein the non-treatment course duration could vary depending on the subjects' state of health and how the subjects respond to the treatment which can be monitored by the said detection of senescent cells in the subject throughout the period of the therapy.

In other embodiments, at least one Senolytic Peptides may be administered in Effective Dose with at least one or more other available senolytic agent which together act additively or synergistically to selectively kill senescent cells.

In all embodiments using the novel senolytic agent(s) on a subject, senescent cell population in the biological sample of the subject undergoing treatment may be monitored before, and after a treatment course. In certain embodiments, said monitoring shall be done during a treatment course and/or between the treatment courses or cycles.

The effectiveness of senolytic Senolytic Peptides treatment can be determined by a person skilled in the medical and clinical arts by employing combination of diagnostic methods, including physical examination, assessment and monitoring of clinical symptoms, and performance of analytical tests and methods described herein, may be used for monitoring the health status of the subject, in addition to the senescent cell monitoring described herein for the treatment regimes. For example in one embodiment the effectiveness of Senolytic Peptides when treating a subject for pulmonary diseases and disorders Pulmonary Function Tests (https://www.nhlbi.nih.gov/health/health-topics/topics/lft) can be conducted. Pulmonary function tests, or PFTs, measure how well subjects' lungs work. They include tests that measure lung size and air flow, such as spirometry and lung volume tests. Other tests measure how well gases such as oxygen get in and out of subjects' blood. These tests include pulse oximetry and arterial blood gas tests. Another pulmonary function test, called fractional exhaled nitric oxide (FeNO), measures nitric oxide, which is a marker for inflammation in the lungs. The subject may have one or more of these tests to diagnose lung and airway diseases, compare subject's lung function to expected levels of function, monitor if your disease is stable or worsening, and see if said senolytic treatment is beneficial.

It should be noted that FoxO4 is expressed in a tissues during fast growth or regeneration therefore any senolytic therapy using the novel senolytic agent(s) should exclude such cohort and should only be directed to post productive aged mammals.

Method of Use of the Senolytic CPPs—Schedule 1

In certain specific embodiments, the senolytic agent is any one of the Senolytic Peptides and in some embodiments, any one of the Senolytic Peptides is administered in a treatment window comprising 11 to 28 days.

In some embodiments, any one of the Senolytic Peptides is administered daily or alternating days for 14 days followed by minimum 14 days off.

In some embodiments, any one of the Senolytic Peptides is administered daily for 13 days followed by minimum 14 days off.

In some embodiments, any one of the Senolytic Peptides is administered daily or alternating days for 12 days followed by minimum 14 days off.

In some embodiments, any one of the Senolytic Peptides is administered daily for 11 days followed by minimum 14 days off.

In some embodiments, any one of the Senolytic Peptides is administered daily or alternating days for 10 days followed by minimum 14 days off.

In some embodiments, any one of the Senolytic Peptides is administered daily for 9 days followed by minimum 14 days off.

In some embodiments, any one of the Senolytic Peptides is administered daily or alternating days for 8 days followed by minimum 12 days off.

In some embodiments, any one of the Senolytic Peptides is administered daily for 7 days followed by minimum 12 days off.

In some embodiments, any one of the Senolytic Peptides is administered daily or alternating days for 6 days followed by minimum 12 days off.

In some embodiments, any one of the Senolytic Peptides is administered daily for 5 days followed by minimum 12 days off.

In some embodiments, any one of the Senolytic Peptides is administered daily or alternating days for 4 days followed by minimum 12 days off.

In some embodiments, any one of the Senolytic Peptides is administered daily for 3 days followed by minimum 10 days off.

In some embodiments, any one of the Senolytic Peptides is administered daily for 2 days followed by minimum 10 days off.

In some embodiments, any one of the Senolytic Peptides is administered for 1 day followed by minimum 10 days off.

In some embodiments, any one of the Senolytic Peptides is administered daily for 14 days in a dose of about 0.5 mg/kg to 20 mg/kg

In some embodiments, any one of the Senolytic Peptides is administered daily for 14 days in a dose of about 0.5 mg/kg to 15 mg/kg

In some embodiments, any one of the Senolytic Peptides is administered daily for 14 days in a dose of about 0.5 mg/kg to 10 mg/kg

In some embodiments, any one of the Senolytic Peptides is administered daily for 14 days in a dose of about 0.5 mg/kg to 5 mg/kg

In some embodiments, any one of the Senolytic Peptides is administered daily for 14 days in a dose of about 0.5 mg/kg to 3 mg/kg

In some embodiments, any one of the Senolytic Peptides is administered daily for 14 days in a dose of about 1400 mg per day.

In some embodiments, any one of the Senolytic Peptides is administered daily for 14 days in a dose of about 1000 mg.

In some embodiments, any one of the Senolytic Peptides is administered daily for 14 days in a dose of about 700 mg.

In some embodiments, any one of the Senolytic Peptides is administered daily for 14 days in a dose of about 200 mg.

In some embodiments, any one of the Senolytic Peptides is administered daily for 14 days in a dose of about 35 mg.

In some embodiments, any one of the Senolytic Peptides is administered daily for 7 days in a dose of about 35 mg to 1400 mg.

In some embodiments, any one of the Senolytic Peptides is administered daily for 7 days in a dose of about 35 mg to 1000 mg.

In some embodiments, any one of the Senolytic Peptides is administered daily for 7 days in a dose of about 35 mg to 700 mg.

In some embodiments, any one of the Senolytic Peptides is administered daily for 7 days in a dose of about 35 mg to 200 mg.

In some embodiments, any one of the Senolytic Peptides is administered daily for 7 days in a dose of about 1400 mg.

In some embodiments, any one of the Senolytic Peptides is administered daily for 7 days in a dose of about 1000 mg.

In some embodiments, any one of the Senolytic Peptides is administered daily for 7 days in a dose of about 700 mg.

In some embodiments, any one of the Senolytic Peptides is administered daily for 7 days in a dose of about 200 mg.

In some embodiments, any one of the Senolytic Peptides is administered daily for 7 days in a dose of about 35 mg.

In other particular embodiments, the above doses are administered daily for 1, 2, 3, 4, 5, or 6 days or 8, 9, 10, 11, 12, 13 days. In other particular embodiments, the above doses are administered alternative daily for 2, 4, or 6 days or 8, 10, 12, 14 days.

In some embodiments the doses described in this Schedule 1 shall be therapeutically sufficient amounts for the administration of any one of the Senolytic Peptides.

In some embodiments the regiment of administration in this Schedule 1 shall be the method of administration of any one of the Senolytic Peptides.

In some embodiments Senolytic Peptides are administered as described in this Schedule 1 together with other known senolytic agent including but not limited to Navitoclax (ABT-263), Fisetin, A133185240, A115546340, Quercetin, Dasatinib, Piperlongumine, 17-AAG (tanespimycin), Geldanamycin 17-DMAG (alvespimycin), Famotidine, Deferoxamine, Mitoxantrane, Lapatinib, Neratinib with therapeutically sufficient doses for each of these analytical agents but strictly in the regiments as described herein this Schedule 1.

TABLE 1
Interaction analysis and binding free energy predictions

| | | # of | FoxO4 |
|---|---|---|---|
| ID | Sequence (H-to-OH) | interactions* | ΔG** (kJ/mol) |
| 7 | PRKGGRRRRAWGRRRRRRRRRRR | 95b, 12hb | −11083.905 +/− 97.351 |
| | RRRAPRKRLTL | | |
| 2 | YGRKKRRQRRRYGRKKRRQRRRY | 10sb, 8hb | −10397.338 ± 153.406 |
| | GRKKRRQRRR | | |
| 3 | RRRRRRRRRRRRRRRRRR | 11sb, 7hb | −8382.126 ± 148.355 |
| 4 | HHHHHHHHHHHHHHHHRRRRRRR | 5sb, 10hb | −7253.424 ± 95.379 |
| | RRRRRRRR | | |
| 5 | RRRRRRRRRRRRRRR | 11sb, 6hb | −7062.33 ± 73.41 |
| 6 | RRRRRRRRRRRRRR | 65b, 15hb | −6695.406 ± 63.557 |
| 7 | RRRRRRRRRRRRGC | 65b, 14hb | −5833.494 ± 78.814 |
| 8 | SGRGKQGGKARAKAKTRSSRAGL | 55b, 15hb | −5702.716 ± 67.716 |
| | QFPVGRVHRLLRKG | | |
| 9 | RRRRRRRRRRR | 3sb, 17hb | −5176.285 ± 39.765 |
| 10 | RNRSRRRRNRSRRR | 4sb, 16hb | −4562.654 ± 95.534 |
| 11 | TRQARRNRRRRWRERQR | 1sb, 17hb | −4350.27 ± 62.707 |
| 12 | RRRRRRRR | 8sb, 13hb | −4092.787 ± 85.567 |
| 13 | RRRRRRRRR | 7sb, 15hb | −4046.129 ± 101.209 |
| 14 | YGRRRRRRRRR | 5sb, 16hb | −4028.576 ± 51.589 |
| 15 | GGRRARRRRRR | 9sb, 8hb | −3955.068 ± 78.066 |
| 16 | YGRRARRRRRR | 8sb, 14hb | −3671.36 ± 97.247 |
| 17 | RLLRLLRRLLRLLRRLLRC | 5sb, 17hb | −3370.825 ± 928.875 |
| 18 | ARRRRCSGSGSGCGSGSGSCGRRRR | 1sb, 17hb | −3015.623 ± 919.598 |
| 19 | RRRRRR | 3sb, 16hb | −2972.75 ± 65.49 |

*PLIP was used to analyze the number of intermolecular interactions before molecular dynamics.
The abbreviation “sb” refers to the number of salt bridge interactions and “hb” refers to the number hydrogen bond interactions.
**Binding free energy was calculated by the MM-PBSA method.

TABLE 2
Foxo3 binding free energy predictions
of top top three peptides

| | Sequence | FoxO3 |
|---|---|---|
| ID | (H-to-OH) | ΔG* (kJ/mol) |
| 1 | PRKGGRRRRAWGRRRRRR | −8685.687 ± 72.948 |
| | RRRRRRRRAPRKRLTL | |
| 2 | YGRKKRRQRRRYGRKKRR | −10186.797 ± 93.583 |
| | QRRRYGRKKRRQRRR | |
| 3 | RRRRRRRRRRRRRRRRRR | −2521.568 ± 87.624 |

*Binding free energy was calculated by the MM-PBSA method.

Additionally or alternatively, in some embodiments the Senolytic Peptide is any amino acid sequence comprising any peptide having at least 70% identity one of to SEQ ID NO: 1 through SEQ ID NO: 19, or having at least 75% identity one of to SEQ ID NO: 1 through SEQ ID NO: 19, or having at least 80% identity one of to SEQ ID NO: 1 through SEQ ID NO: 19, or having at least 85% identity one of to SEQ ID NO: 1 through SEQ ID NO: 19, or having at least 90% identity one of to SEQ ID NO: 1 through SEQ ID NO: 19, or having at least 95% identity one of to SEQ ID NO: 1 through SEQ ID NO: 19, or having at least 96% identity one of to SEQ ID NO: 1 through SEQ ID NO: 19, or having at least 97% identity one of to SEQ ID NO: 1 through SEQ ID NO: 19, or having at least 98% identity one of to SEQ ID NO: 1 through SEQ ID NO: 19, or having at least 99% identity one of to SEQ ID NO: 1 through SEQ ID NO: 19.

Additionally or alternatively, in some embodiments the Senolytic Peptide is any amino acid sequence comprising any peptide deviating by not more than 10 amino acids from one of to SEQ ID NO: 1 through SEQ ID NO: 19, or deviating by not more than 9 amino acids from one of to SEQ ID NO: 1 through SEQ ID NO: 19, or deviating by not more than 8 amino acids from one of to SEQ ID NO: 1 through SEQ ID NO: 19, or deviating by not more than 7 amino acids from one of to SEQ ID NO: 1 through SEQ ID NO: 19, or deviating by not more than 6 amino acids from one of to SEQ ID NO: 1 through SEQ ID NO: 19, or deviating by not more than 5 amino acids from one of to SEQ ID NO: 1 through SEQ ID NO: 19, or deviating by not more than 4 amino acids from one of to SEQ ID NO: 1 through SEQ ID NO: 19, or deviating by not more than 3 amino acids from one of to SEQ ID NO: 1 through SEQ ID NO: 19, or deviating by not more than 2 amino acids from one of to SEQ ID NO: 1 through SEQ ID NO: 19, or deviating by not more than 1 amino acid from one of to SEQ ID NO: 1 through SEQ ID NO: 19.

In some embodiments, the Senolytic Peptide is any amino acid sequence comprising any peptide one of SEQ ID NO: 1, alternatively, any amino acid sequence comprising any peptide one of SEQ ID NO: 2, alternatively, any amino acid sequence comprising any peptide one of SEQ ID NO: 3, alternatively, any amino acid sequence comprising any peptide one of SEQ ID NO: 4, alternatively, any amino acid sequence comprising any peptide one of SEQ ID NO: 5, alternatively, any amino acid sequence comprising any peptide one of SEQ ID NO: 6, alternatively, any amino acid sequence comprising any peptide one of SEQ ID NO: 7, alternatively, any amino acid sequence comprising any peptide one of SEQ ID NO: 8, alternatively, any amino acid sequence comprising any peptide one of SEQ ID NO: 9, alternatively, any amino acid sequence comprising any peptide one of SEQ ID NO: 10, alternatively, any amino acid sequence comprising any peptide one of SEQ ID NO: 11 alternatively, any amino acid sequence comprising any peptide one of SEQ ID NO: 12, alternatively, any amino acid sequence comprising any peptide one of SEQ ID NO: 13, alternatively, any amino acid sequence comprising any peptide one of SEQ ID NO: 14, alternatively, any amino acid sequence comprising any peptide one of SEQ ID NO: 15, alternatively, any amino acid sequence comprising any peptide one of SEQ ID NO: 16, alternatively, any amino acid sequence comprising any peptide one of SEQ ID NO: 17, alternatively, any amino acid sequence comprising any peptide one of SEQ ID NO: 18, alternatively, any amino acid sequence comprising any peptide one of SEQ ID NO: 19.

In some embodiments, the Senolytic Peptide(s) may be characterized with respect to mutations to one of to SEQ ID NO: 1 through SEQ ID NO: 19, wherein the mutations enhance the binding of the peptide to FoxO4 and/or reduce the binding of the peptide to other FoxO proteins or DNA, or p53. In some embodiments, the mutations present at these positions include one of the amino acids: A, C, D, E, F, H, I, K, L, N, R, Q, S, T or Y, for example, so as to enhance the binding affinity of the peptide to FoxO4. Additionally or alternatively, in some embodiments, the Senolytic Peptides include changes (e.g., mutations) at the critical positions to non-natural amino acids or peptidomimetics can be used to mimic charged amino acids for the numbered positions.

In other embodiments, the Senolytic Peptides can be shortened from the N- and/or C-termini in order to promote stability. In other embodiments, any of the Senolytic Peptides can be expressed in the fusion forms with other Senolytic Peptides or other peptides that are not associated with senolytic activity.

In some embodiments, the Senolytic Peptide is the unique artificial peptide comprising all or substantially all D-amino acids or a mixture of D- and L-amino acids. Additionally or alternatively, in some embodiments, the Senolytic Peptide(s) is expressed by addition of single or multiple D-amino acids to the N- and/or C-termini, for example, to increase the bioavability of the peptide. For example, in some embodiments, peptides with D-amino acids can mimic L-peptides and modification to D-retroinverso (DRI) forms often change the peptide to a more potent form in vitro and in vivo. These DRI peptide forms have also been shown to be therapeutically effective in clinical trials. As will be recognized by those skilled in the art the one or more of the peptides shown in the SEQUENCE LISTING as SEQ ID NO: 1 through SEQ ID NO: 19, could be expressed by using D-amino acids and in a retroreversed sequence. In some embodiments, the Senolytic Peptide(s) is expressed by using D-amino acids and in a retroreversed sequence.

In some embodiments, the Senolytic Peptide(s) is administered for therapeutic, symptom reducing, or at least partially preventative use against senescence-related pathologies.

In some embodiments, the Senolytic Peptide(s) is suitably administered by fusing to a peptide that facilitates the cell entry from N- or C-terminal. For example, when used as a senolytic agents the Senolytic Peptide(s) need to be enabled to enter into a mammalian cell. Peptides, often called cell-penetrating peptides (CPPs) or protein transduction domains (PTDs), are non-invasive vectors that can efficiently transport various biologically active molecules inside cells. These peptides can transport cargoes ranging from peptides to nanoparticles through a covalent or non-covalent linkage. The transport of the smallest cargo to large 120 kDa proteins was shown to be successful both in vitro and in vivo. Moreover, activable CPPs (ACPPs) have recently reported to perform targeted delivery to cancer cells that overexpress metalloproteinase-2.

In some embodiments of the Senolytic Peptide(s) is expressed using peptidomimetics. For example, peptidomimetics are chemical compounds that may be effective to mimick a natural peptide and to interact with a target so as to exert the same biological effect. Peptidomimetics are often recruited to overcome problems associated with the intrinsic properties of natural peptides such as stability against proteolytic degradation. Peptide bond surrogates and β-turn dipeptide mimetics among examples of peptidomimetics.

In some embodiments, the Senolytic Peptide(s) could be expressed in a cyclic form. For example, in some applications, cyclic peptides may be more effective than their linear counterparts, for example, due to the conformational rigidity. Moreover peptides having a cyclic structure may be relatively resistant to proteolytic cleavage by exopeptidases due to the termini and by endopeptidases as their structure is more rigid than linear peptides.

In some embodiments, the Senolytic Peptide(s) could be expressed as a product of polynucleotides. For example, in some embodiments, polynucleotides that are complementary to at least a portion of a nucleotide sequence encoding the Senolytic Peptide(s) (e.g., a short interfering nucleic acid, an antisense polynucleotide or a peptide nucleic acid) may be prepared using the peptide sequences available in the art to alter gene and/or protein expression. These polynucleotides may specifically bind to or hybridize to certain nucleic acid molecules.

In some embodiments, the polynucleotides may be delivered by a recombinant vector in which the polynucleotide of interest has been incorporated. Additionally or alternatively, in some embodiments, the recombinant viral vector may be a recombinant expression vector into which a polynucleotide sequence that encodes the designed peptides has been incorporated. The recombinant vector or the recombinant expression vector may be a viral recombinant vector or a viral recombinant expression vector. As will be recognized by those skilled in the art the Senolytic Peptide(s) could be expressed via a vector.

In some embodiments, the Senolytic Peptide(s) may comprise one or more unnatural or unusual amino acids, for example, that are not naturally encoded amino acids. These may be non-proteinogenic amino acids which can occur naturally in plant or bacteria post-translationally or are chemically synthesized as pharmacological motifs. Unnatural amino acids are widely used in combinatorial libraries or as chiral building blocks. These modified amino acids often contribute to special biological activity, and are often incorporated into therapeutic peptidomimetic ligands to enhance peptide pharmacological activity and potency. Unusual or unnatural amino acid modified peptides can improve selectivity, receptor binding, modify activity (agonists and antagonists), increase in vivo half-life, enhance transport across cell membranes. As will be recognized by those skilled in the art, the disclosed CPPs are expressed by a wide range of unnatural, non-standard amino acid peptide modifications. In some embodiments, modifications to CPPs include incorporation of unnatural/unusual amino acids into the sequence using D-amino acids, L-amino acids, N-methyl amino acids, homo amino acids, alpha-methyl amino acids, beta (homo) amino acids, gamma amino acids, helix/turn stabilizing compound or backbone modification peptoides. Additionally or alternatively, in some embodiments, CPP sequence(s) are modified by insertion of natural occurring unusual amino acids such as citrulline (Cit), hydroxyproline (Hyp), beta-alanine, ornithine (Orn), norleucine (Nle), 3-nitrotyrosine, pyroglutamic acid (Pyr), nitroarginine, Alanine Modified Amino Acid Analogs, Alicyclic Amino Acids Analogs, Aromatic Amino Acids Analogs, Aspartic Acid Analogs, Beta-Amino Acids Analogs, Cysteine Amino Acid Analogs, DAB (2,4-Diaminobutyric Acid) Analogs, DAP (2,3-Diaminopropionic Acid) Analogs, Depsipeptides, Glutamic Acid Amino Acid Analogs, Glutamine Amino Acid Analogs, Glycine Amino Acid Analogs, Heterocyclic Amino Acid Analogs, Histidine Amino Acid Analogs, Homo-Amino Acid Analogs, Isoleucine Amino Acid Analogs, Leucine Amino Acid Analogs, Linear Core Amino Acid Analogs, Lysine Amino Acid Analogs, Methionine Amino Acid Analogs, N-Methyl Amino Acids Analogs, Norleucine Amino Acid Analogs, Norvaline Amino Acid Analogs, Ornithine Amino Acid Analogs, Statine Amino Acid Analogs, Penicillamine Amino Acid Analogs, PEGylated Amino Acids, Phenylalanine Amino Acid Analogs, Phenylglycine Amino Acid Analogs, Proline Amino Acid Analogs, Pyroglutamine Amino Acid Analogs, Serine Amino Acid Analogs, Threonine Amino Acid Analogs, Tryptophan Amino Acid Analogs, Tyrosine Amino Acid Analogs, Valine Amino Acid Analogs, Post-Translational Modification (PTM) Modified Amino Acids, Deiminated Arginines (Citrulline), DL-Citrulline, L-Citrulline, Methylated Aminod Acids, Lysine(Me), Lysine(Me2), Lysine(Me3), Arginine(Me), Arginine(Me)2 asymmetrical, Arginine(Me)2 symmetrical, Phosphorylated Amino Acids, Phosphoserine, phosphothreonine, phosphotyrosine.

In various embodiments, the Senolytic Peptide(s), for example, one or more of SEQ ID NO: 1 through SEQ ID NO: 19 as shown in the SEQUENCE LISTING or derivatives thereof, for example, peptides containing not more of 30% mutations with respect to these sequences, may also interfere with CR3 domain of FoxO4 thereby interfering with SCAP. Furthermore, the Senolytic Peptide(s) that are formed by combining or shuffling some part of one or more peptide sequences that is at least 6 amino acid in length shall carry the CR3 domain binding affinity thereby interfering with FoxO4.

In some embodiments, methods for selectively inducing apoptosis in (e.g., killing) senescent cells in a subject who has a senescence-associated disease or disorder may generally comprise administering one or more of the Senolytic Peptide(s) to the subject in need thereof, for example, according to one or more of the administration methods described herein. For example, in some embodiments, the method may comprise causing an artificial peptide comprising an amino acid sequence having at least 90% identity to any one of SEQ ID NO: 1 through SEQ ID NO: 19 to interfere with the CR3 domain of Forkhead box protein 04 (FoxO4) of the senescent cell.

Method of Use of the Senolytics

The proportion of senescent cells in a tissue of a mammal varies with the biological age and could vary substantially depending on the cohort that mammal subject belongs to. Moreover, the proportion of senescent cells present may further vary with the type of the tissue in a given subject. These variations may create a challenge in specifying a dose for the Senolytic Agent for a rejuvenation therapy. Additional complexities in specifying a dose arise when said senescent cell proportion is above a certain threshold and therefore accelerated apoptosis could result in frailty. Fortunately, unlike for cancer interventions, complete elimination of senescent cells may not be necessary for achieving beneficial effects.

In various embodiments, the Senolytic Peptides may be administered via any suitable methodology. By way of non-limiting example, disclosed herein three different treatment regimes, namely an Impulse Regime, a Sustained Regime, and Gentle Regime via which the Senolytic Peptide(s) may be administered.

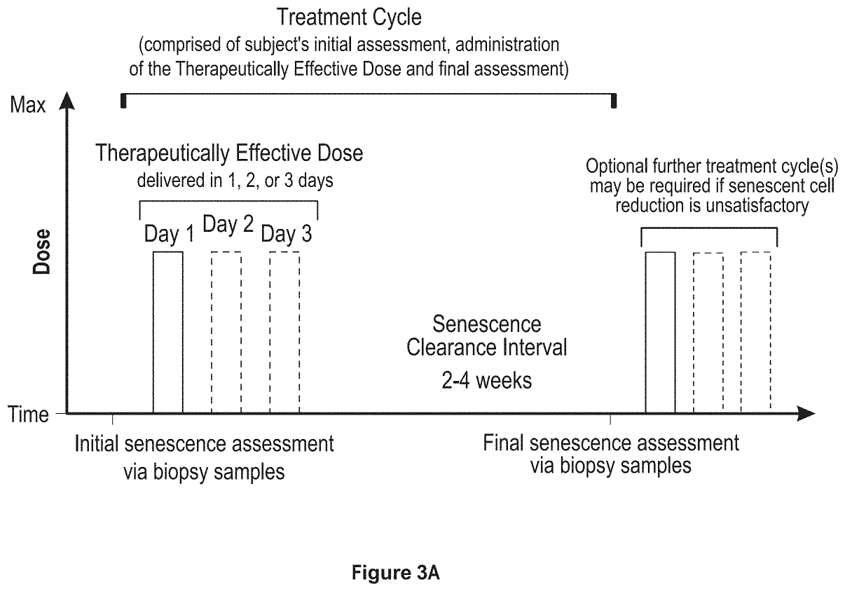

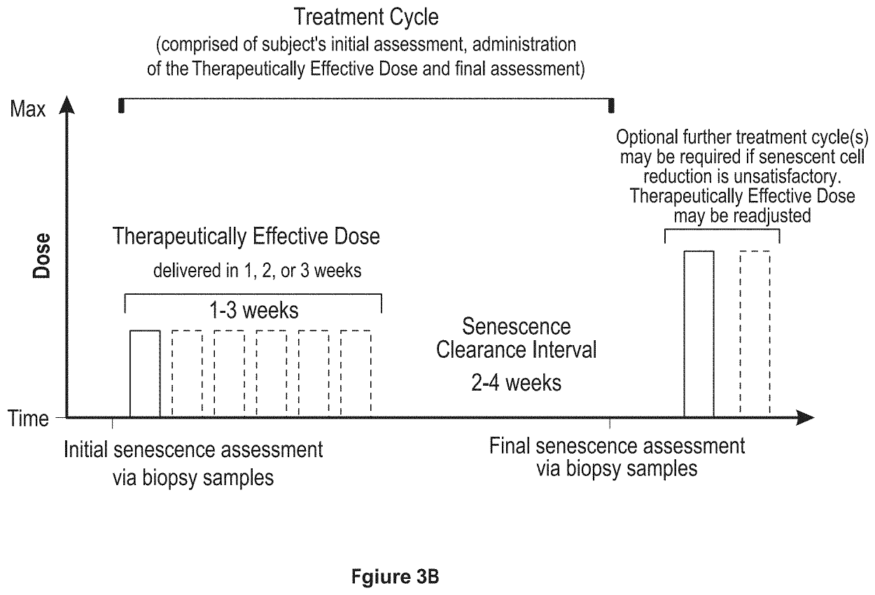

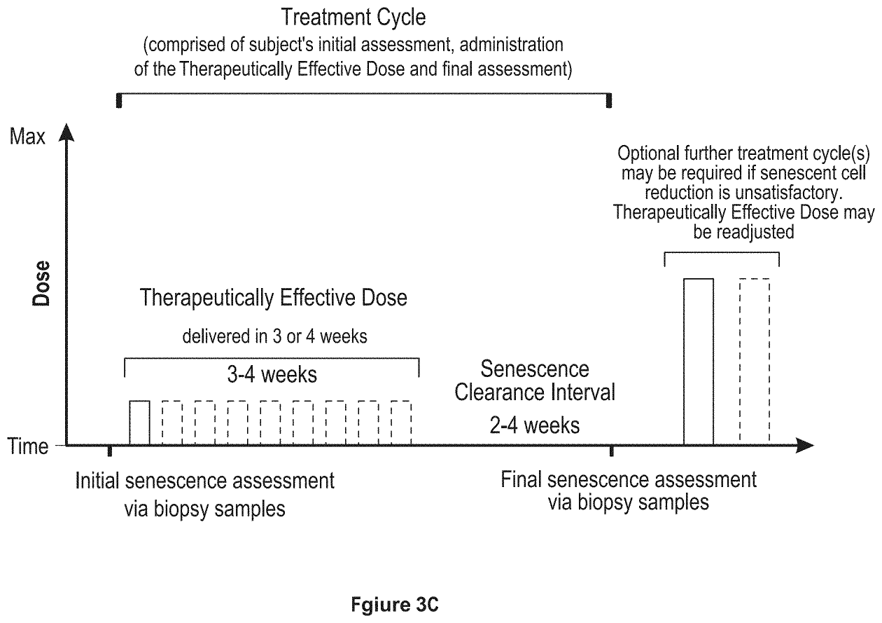

In some embodiments, a method is provided for treating a senescence-associated disease or disorder comprising administering to a subject in need thereof a therapeutically Effective Dose of the Senolytic Peptide(s), for example, the Senolytic Peptide(s) may be administered intermittently in one or multiple treatment cycles. Each treatment cycle may extend over 1 or 2 or 3 days. Each administration may include equivalent doses adjusted so as to cumulatively reach the therapeutically Effective Dose at the end of each treatment cycle. In some embodiments, the therapeutically Effective Dose is administered equivalently in a single day or intermittently in two consecutive days or in three consecutive days or 2 or 3 administration in alternate days. This regime of administration of the Senolytic Peptides is referred to herein as the Impulse Regime. In the Impulse Regime (FIG. 3A), the therapeutically Effective Dose is achieved through one or two subsequent administration(s). In some embodiments, after the first cycle, there may be a two week-four-week senescence clearance interval, for example, allowing a period of time effective for a decrease in senescent cells. In some embodiments, the subject is evaluated, both before the Impulse Regime and after Senescence Clearance Interval period, by one skilled in the art to determine levels of various SASP markers for determination of the therapeutically Effective Dose and Follow-up Treatment, respectively.

Additionally, or alternatively, in some embodiments, the therapeutically Effective Dose is achieved through a single or multiple administrations in a period of 1-3 weeks. The quantity of the Senolytic Peptide for each administration is equivalent and adjusted to cumulatively reach the therapeutically Effective Dose at the end of the treatment. This regime of administration of the Senolytic Peptides is referred to as the Sustained Regime. The therapeutically Effective Dose administered in the Sustained Regime may be higher than the therapeutically Effective Dose as would be administered in the Impulse Regime.

Additionally, or alternatively, in some embodiments the therapeutically Effective Dose is administered intermittently in one or multiple treatment cycles wherein each treatment cycle comprised of 1 or 2 or 3 or 4 or 5 or 6 administration days equally distributed in 1-3 weeks where each administration is in equivalent doses adjusted to cumulatively reach the therapeutically Effective Dose at the end of each treatment cycle.

In some embodiments, after the first cycle, there is a two or three weeks' senescence clearance interval allowing a period of time effective for a decrease in senescent cells. In some embodiments, the subject may be evaluated, both before the Sustained Regime and after Senescence Clearance Interval period, by one skilled in the art based on the levels of various SASP markers for determination of the therapeutically Effective Dose and Follow-up Treatment, respectively.

In some embodiments, the therapeutically Effective Dose is achieved through single or multiple administration cycles in the period of 3-4 weeks. This regime of administration of the Senolytic Peptides is referred to herein as the Gentle Regime. Both the therapeutically Effective Dose and the quantity of the Senolytic Peptide for a single administration in the Gentle Regime may be lower than those in the Impulse Regime and Sustained Regime. The therapeutically Effective Dose may be administered intermittently in one or multiple treatment cycles. Each treatment cycle may be comprised of 1 or 2 or 3 or 4 or 5 or 6 or 7 or 8 or 9 administration days equally distributed in 3-4 weeks. Each administration may be in an equivalent dose adjusted so as to cumulatively reach the therapeutically Effective Dose at the end of each treatment cycle.

In some embodiments, after the first treatment cycle, there is a two or three weeks Senescence Clearance Interval allowing a period of time for the treatment becoming effective with gradual decrease in senescent cells. In some embodiments, the subject may be evaluated both before the Gentle Regime and after Senescence Clearance Interval period, by those skilled in the art based on the levels of various SASP markers for determination of the therapeutically Effective Dose and Follow-up Treatment, respectively.

In various embodiments, the therapeutically Effective Dose amount is delivered to a subject in need thereof by any one of several routes known to a person skilled in the art. In various embodiments, by way of non-limiting example, the therapeutically Effective Dose is delivered orally, intravenously, intraperitoneally, by infusion (e.g., a bolus infusion), subcutaneously, enteral, rectal, intranasal, by injection, inhalation, buccal, sublingual, intramuscular, transdermal, intradermal, topically, intraocularly, vaginally, rectally, intrathecally or by intracranial injection, or by directly into the target tissue including a tumor or organ, or subcutaneous route or by some combination of the above thereof. In some embodiments the therapeutically Effective Dose may be delivered in combinations of other Senolytic Agents and/or with chemotherapy. In some embodiments the therapeutically Effective Dose may be delivered in combinations of other Senolytic Agents and/or with chemotherapy concomitantly or concurrently or not concurrently but in appropriate intervals. In some embodiments, a delivery method includes controlled or sustained release drug(s), drug-coated or permeated stents for which the drug is the Senolytic Peptide. In some embodiments therapeutically Effective Dose amount is further adjusted depending on the delivery route and selected Treatment Regime.

As discussed herein below, examples 1-5 indicate that the Senolytic Peptide(s) can safely be administered at doses of 5 mg/Kg, 10 mg/Kg, and 15 mg/Kg. More particularly, Example 4 indicates that the Senolytic Peptide(s) is non-toxic at levels up to 100 mg/kg. Due to the molecular similarity of the various Senolytic Peptides in the Table 1 and peptides having at least 90% identity thereto, atomistic structures are similar and therefore they exert their senolytic effects in a similar way. In some embodiments, the therapeutically Effective Dose amount can further depend upon the patient's height, weight, sex, age and medical history.

In some embodiments, treatment methods, such as according to the Impulse Regime, Sustained Regime, or Gentle Regime, may include monitoring the population of the senescent cells on convenient biological samples taken from the subject at the beginning and 2 or 4 weeks after the treatment to determine the effectiveness of the therapy and or whether a second or a third treatment course is required. In some embodiments, an individualized treatment course may be implemented and may include monitoring of senescent cell population at the beginning of a therapy and a certain period after each treatment course and adjusting the treatment course or the dose or the treatment regime. Additionally, or alternatively, in some embodiments the administration of a therapeutically Effective Dose may depend upon the subject's state of health and subject's response to the treatment throughout the period of the therapy. In some embodiments, the biological sample shall be the skin biopsy specimens obtained from skin tissue of the subject is collected with minimally invasive methods. In some embodiments, the detection of the senescent cells may be achieved using senescence associated markers

Although senescent cells diverge from other quiescent and terminally differentiated cells, they do not display a unique phenotype but a variety of phenotypes which define the senescent state. Hallmarks of the senescent cells in this state include permanent and irreversible growth arrest; increase in cell size; expression of senescence-associated beta-galactosidase (SA-β-Gal) enzyme resulting in increased lysosomal content; expression of a tumor suppressor, p16^{INK4a}. Two biomarkers have been extensively used for identification of senescent cells:
SA-β-Gal: Enzyme activity assayed at pH 6 using X-Gal as the substrate.
p16^{INK4a}: Monitoring the expression levels of p16^{INK4a} protein which is a CDK4/6 inhibitor and is involved in maintenance of growth arrest.

In some embodiments, the Senolytic Peptide(s) do not have to be continuously present to exert an intended effect. For example, brief disruption of pro-survival pathways is adequate to kill senescent cells and/or circulating tumor cells (CTCs) and/or dormant cells and/or cancer stem cells. Thus the Senolytic Peptide(s) can be effective as Senolytic Peptides when administered intermittently.

It has already been reported that other known Senolytic Agents such as the tyrosine kinase inhibitor (D) and the flavonoid, quercetin (Q), were shown to induce apoptosis in senescent cells. Intermittent administration of D+Q alleviated frailty, neurological dysfunction, osteoporosis, and vertebral disk degeneration related to loss of glycosaminoglycans on an accelerated aging-like state. Furthermore, in mice with impaired mobility due to radiation of one of their legs 3 months previously, treadmill endurance improved within 4 days after completing a single course of D+Q. Said improvement persisted for at least 7 months. D+Q has an elimination half-life of a few hours. These outcomes following intermittent or single courses of agents with short elimination half-lives are consistent with the long-lasting type of effect expected from reducing senescent cell abundance, as opposed to what would be expected if D+Q had to be continuously present to suppress or activate cellular processes by occupying a receptor or acting on an enzyme.

Thus, intermittent rather than continuous treatment with Senolytic Agents may be effective in alleviating senescence-related diseases or disorders, allowing these agents to be administered during periods of good health and potentially decreasing risk of side-effects. In all of the shock dose regimes, there will be an optional follow-up treatment wherein the therapeutically Effective Dose can be increased or decreased. In between the initial treatment and follow-up treatment there will be a non-treatment interval, wherein the non-treatment course duration could vary depending on the subject's state of health and/or the subject's response to the treatment which can be monitored by the said detection of senescent cells in the subject throughout the period of the therapy.

In some embodiments, at least one Senolytic Peptide may be administered in a therapeutically Effective Dose with at least one or more other available Senolytic Agent which together act additively or synergistically to selectively kill senescent cells. By way of non-limiting example, in some embodiments, a Senolytic Peptide dose administration is optimization for use with the Senolytic Agents including but not limited to Dasatinib, Quercetin, Navitoclax, Piperlongumin, Fisetin, BCL-XL inhibitors A1331852 and A1155463, FOXO4-related peptide, Foxo4-CR3 domain inhibiting peptides or combinations of these.

The effectiveness of the Senolytic Peptide treatment can be determined by a person skilled in the medical and clinical arts by employing combination of diagnostic methods, including physical examination, assessment and monitoring of clinical symptoms, and performance of analytical tests and methods described herein, may be used for monitoring the health status of the subject, in addition to the senescent cell monitoring described herein for the Treatment Regimes. For example in one embodiment the effectiveness of the Senolytic Peptides when treating a subject for pulmonary diseases and disorders Pulmonary Function Tests (https://www.nhlbi.nih.gov/health/health-topics/topics/lft) can be conducted. Pulmonary function tests, or PFTs, measure how well subject's lungs work. They include tests that measure lung size and air flow, such as spirometry and lung volume tests. Other tests measure how well gases such as oxygen get in and out of subject's blood. These tests include pulse oximetry and arterial blood gas tests. Another pulmonary function test, called fractional exhaled nitric oxide (FeNO), measures nitric oxide, which is a marker for inflammation in the lungs. The subject may have one or more of these tests to diagnose lung and airway diseases, compare subject's lung function to expected levels of function, monitor if your disease is stable or worsening, and see if said senolytic treatment is beneficial.

It should be noted that FoxO4 is expressed in a tissues during fast growth or regeneration. As such, in some embodiments a senolytic therapy using the Senolytic Peptide(s) may exclude such cohorts.

In some embodiments using the Senolytic Peptide(s) on a subject, senescent cell population in the Biological skin sample of the subject undergoing treatment will be monitored before, and after a treatment course. In some embodiments, monitoring is performed during a treatment course and/or between the treatment courses or cycles.

Method of Use of the Senolytic Agents—Schedule 1

In some embodiments, the Senolytic Agent comprises any one of the Senolytic Peptides or combinations Senolytic Agents.

In some embodiments, the Senolytic Agent is administered in a treatment window comprising 11 to 28 days.

By way of non-limiting example, in some embodiments, the Senolytic agent is administered daily or alternating days for 14 days followed by minimum 14 days off.

In some embodiments, the Senolytic Agent is administered daily for 13 days followed by minimum 14 days off.

In some embodiments, the Senolytic Agent is administered daily or alternating days for 12 days followed by minimum 14 days off.

In some embodiments, the Senolytic Agent is administered daily for 11 days followed by minimum 14 days off.

In some embodiments, the Senolytic Agent is administered daily or alternating days for 10 days followed by minimum 14 days off.

In some embodiments, the Senolytic Agent is administered daily for 9 days followed by minimum 14 days off.

In some embodiments, the Senolytic Agent is administered daily or alternating days for 8 days followed by minimum 12 days off.

In some embodiments, the Senolytic Agent is administered daily for 7 days followed by minimum 12 days off.

In some embodiments, the Senolytic Agent is administered daily or alternating days for 6 days followed by minimum 12 days off.

In some embodiments, the Senolytic Agent is administered daily for 5 days followed by minimum 12 days off.

In some embodiments, the Senolytic Agent is administered daily or alternating days for 4 days followed by minimum 12 days off.

In some embodiments, the Senolytic Agent is administered daily for 3 days followed by minimum 10 days off.

In some embodiments, the Senolytic Agent is administered daily for 2 days followed by minimum 10 days off.

In some embodiments, the Senolytic Agent is administered for 1 day followed by minimum 10 days off.

In some embodiments, the Senolytic Agent is administered daily for 14 days in a dose of about 0.1 mg/kg to 20 mg/kg.

In some embodiments, the Senolytic Agent is administered daily for 14 days in a dose of about 0.1 mg/kg to 15 mg/kg.

In some embodiments, the Senolytic Agent is administered daily for 14 days in a dose of about 0.1 mg/kg to 10 mg/kg.

In some embodiments, the Senolytic Agent is administered daily for 14 days in a dose of about 0.1 mg/kg to 5 mg/kg.

In some embodiments, the Senolytic Agent is administered daily for 14 days in a dose of about 0.1 mg/kg to 3 mg/kg.

In some embodiments, the Senolytic Agent is administered daily for 14 days in a dose of about 0.1 mg/kg to 0.5 mg/kg.

In some embodiments, the Senolytic Agent is administered daily for 14 days in a dose of about 1400 mg per day.

In some embodiments, the Senolytic Agent is administered daily for 14 days in a dose of about 1000 mg.

In some embodiments, the Senolytic Agent is administered daily for 14 days in a dose of about 700 mg.

In some embodiments, the Senolytic Agent is administered daily for 14 days in a dose of about 200 mg.

In some embodiments, the Senolytic Agent is administered daily for 14 days in a dose of about 35 mg.

In some embodiments, the Senolytic Agent is administered daily for 14 days in a dose of about 3.5 mg.

In some embodiments, the Senolytic Agent is administered daily for 7 days in a dose of about 3.5 mg to 1400 mg.

In some embodiments, the Senolytic Agent is administered daily for 7 days in a dose of about 3.5 mg to 1000 mg.

In some embodiments, the Senolytic Agent is administered daily for 7 days in a dose of about 3.5 mg to 700 mg.

In some embodiments, the Senolytic Agent is administered daily for 7 days in a dose of about 3.5 mg to 200 mg.

In some embodiments, the Senolytic Agent is administered daily for 7 days in a dose of about 3.5 mg to 35 mg.

In some embodiments, the Senolytic Agent is administered daily for 7 days in a dose of about 1400 mg.

In some embodiments, the Senolytic Agent is administered daily for 7 days in a dose of about 1000 mg.

In some embodiments, the Senolytic Agent is administered daily for 7 days in a dose of about 700 mg.

In some embodiments, the Senolytic Agent is administered daily for 7 days in a dose of about 200 mg.

In some embodiments, the Senolytic Agent is administered daily for 7 days in a dose of about 35 mg.

In some embodiments, the Senolytic Agent is administered daily for 7 days in a dose of about 3.5 mg.

In some embodiments, the Senolytic Agent is administered in one of the above doses daily for 1, 2, 3, 4, 5, or 6 days or 8, 9, 10, 11, 12, 13 days. Additionally, or alternatively, in some embodiments, the Senolytic Agent is administered in one of the above doses are administered alternative daily for 2, 4, or 6 days or 8, 10, 12, 14 days.

In some embodiments the doses described in this Schedule 1 shall be a therapeutically sufficient dose for the administration of any one of the Senolytic agents.

In some embodiments therapeutically Effective Dose is administered by any one of several compositions and delivery routes known to a person skilled in the art and as disclosed in the subject matter. By way of non-limiting example, the composition may be delivered orally, intravenously, intraperitoneally, by infusion (e.g., a bolus infusion), subcutaneously, enteral, rectal, intranasal, by injection, inhalation, buccal, sublingual, intramuscular, transdermal, intradermal, topically, intraocularly, vaginally, rectally, intrathecally, or intracranially, or by some combination thereof.

In some embodiments the regiment of administration in this Schedule 1 shall be the method of administration of any one of the Senolytic agents.

In some embodiments, the therapeutically Effective Dose is delivered in methods of the disclosed subject matter including but limited to as in controlled or sustained release drug(s), drug-coated or permeated stents for which the drug is the Senolytic Agent.

In some embodiments Senolytic Peptide(s) is administered as described in this Schedule 1 together with another Senolytic Agent including but not limited to Navitoclax (ABT-263), Fisetin, A133185240, A115546340, Quercetin, Dasatinib, Piperlongumine, 17-AAG (tanespimycin), Geldanamycin 17-DMAG (alvespimycin), Famotidine, Deferoxamine, Mitoxantrane, Lapatinib, Neratinib with therapeutically sufficient doses for each of these Senolytic Agents but strictly in the regiments as described herein this Schedule 1.

The effectiveness of the Senolytic Therapy can be determined by a person skilled in the medical and clinical arts by employing combination of diagnostic methods, including physical examination, assessment and monitoring of clinical symptoms, and performance of analytical tests and methods described herein, may be used for monitoring the health status of the subject, in addition to the monitoring described herein for the treatment regimes.

Medical Therapies Using the Senolytic Peptides

The effectiveness of the Senolytic Peptide(s) with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, subject self-assessment, assessment and monitoring of clinical symptoms, performance of analytical tests and methods, including clinical laboratory tests, physical tests, and exploratory surgery, for example, may be used for monitoring the health status of the subject and the effectiveness of the Senolytic Peptide. The effects of the methods of treatment described herein can be analyzed using techniques known in the art, such as comparing symptoms of subjects suffering from or at risk of a particular disease or disorder that have received the composition comprising the Senolytic Peptide with those of subjects who were not treated with the Senolytic Peptide or who received a placebo treatment.

A subject in need of treatment with the Senolytic Peptide(s) as described herein may be a human or may be a non-human primate or other animal (i.e., veterinary use) who has developed symptoms of a senescence cell-associated disease or disorder or who is at risk for developing a senescence cell-associated disease or disorder. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, elephants, bears and other domestic, farm, and zoo animals.

In some embodiments, administration of the Senolytic Peptide(s) described herein can prolong survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving the Senolytic Peptide would provide clinical benefit to delay development or reduce severity of a disease, including an age-related disease or disorder.

In some embodiments, use of the Senolytic Peptides may be restricted during wound healing (including pre- or post-operations). When a wound is present, senescent cells may be induced around the wound. Senescent cells make growth factors that are required for wound healing. However, this innate mechanism is not disturbed unless the Senolytic Peptide(s) is administered at the time of the wound healing.

Method of Use of the Senolytic Peptides in Senescence-Associated Diseases and Disorders

Cellular senescence is a cell fate that involves essentially irreversible replicative arrest, apoptosis resistance, and frequently increased protein synthesis, metabolic shifts with increased glycolysis, decreased fatty acid oxidation, increased reactive oxygen species generation, and acquisition of a senescence-associated secretory phenotype (SASP).

Methods are provided herein for treating conditions, diseases or disorders related to, associated with, or caused by cellular senescence in a subject in need thereof.

In some embodiments the methods of use of Senolytic Agents for treatment of senescence-associated diseases and disorders comprise Senolytic Therapy as disclosed in the subject matter of this patent. Some examples of Treatment Regimes of Senolytic Therapy for treatment of senescence-associated diseases and disorders include administering therapeutically Effective Dose via Impulse Regime, Sustained Regime, and Gentle Regime or via any of the regimes disclosed in Schedule 1. In various embodiments of the Senolytic Therapy for treatment of senescence-associated diseases and disorders certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method of Use of the Senolytic Peptide(s) for Treatment of General Age-Related Diseases and Disorders

In some embodiments, the Senolytic Peptide(s) inhibits senescence of adult stem cells or inhibits accumulation, kills, or otherwise facilitates removal of adult stem cells that have become senescent. Therefore, the Senolytic Peptides may also be useful for treating or preventing of an age-related disease or disorder that occurs as part of the natural aging process or that occurs when the subject is exposed to a senescence inducing agent or factor (e.g., irradiation, chemotherapy, smoking tobacco, high-fat/high sugar diet, other environmental factors).

In some embodiments, frailty as an aging-associated decline may be treated or prevented (i.e., the likelihood of occurrence of is reduced) by administering the Senolytic Peptide. The effectiveness of the senolytic therapy can be measured by monitoring the frailty index of the patient.

In some embodiments, the age related disease or disorder is scoliosis. Effectiveness of the senolytic therapy are measured by, inter alia, physical examination of spine, ribs, hips and shoulders and/or X-RAY, CT and/or MRI to determine bone curvature.

In some embodiments the methods of use of Senolytic Agents for treatment of general age-related diseases and disorders comprise Senolytic Therapy as disclosed in the subject matter of this patent. Some examples of said Treatment Regimes for age-related diseases and disorders include administering therapeutically Effective Dose via Impulse Regime, Sustained Regime, and Gentle Regime or via any of the regimes disclosed in Schedule 1. In various embodiments of the Senolytic Therapy for treatment of general age-related diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population and/or circulating tumor cells (CTCs) and/or dormant cells and/or cancer stem cells on a biopsy sample taken from the subject at the beginning and throughout the period of the therapy and thus monitoring the target cell population decline.

The effectiveness of a method of treatment described herein may be manifested by reducing the number of symptoms of an age-related disorder or age-sensitive trait associated with a senescence-inducing stimulus, decreasing the severity of one or more symptoms, or delaying the progression of an age-related disorder or age-sensitive trait associated with a senescence-inducing stimulus. In some embodiments, preventing an age-related disorder or age-sensitive trait associated with a senescence-inducing stimulus refers to preventing (i.e., reducing the likelihood of occurrence) or delaying onset of an age-related disorder or age-sensitive trait associated with a senescence-inducing stimulus, or reoccurrence of one or more age-related disorder or age-sensitive trait associated with a senescence-inducing stimulus.

Method Use of the Senolytic Peptide(s) in Tissue Rejuvenation

It has been reported in the reference that a Senolytic Agent could influence the health span of mice in which senescence and the concomitant loss of tissue homeostasis were allowed to develop spontaneously as a consequence of aging. The results of in vivo mouse experiments indicate that a Senolytic Agent can reduce cellular senescence and counteract hair loss (objectively measured as the fur density) and general frailty (objectively measured by increased running wheel activity or muscle size increase) in mice.

Tissues contain high levels of senescent cells, which due to chronic SASP secretion, would inflict permanent reprogramming on their neighboring cells. Senescence was recently shown to trigger tissue reprogramming in vivo, leading to Nanog-positive cells in the vicinity of areas of senescence. Senescent cells might thus trigger reprogramming of neighboring cells into more pluripotent cells. However, since the release of SASP factors (such as IL6) is continuous, they would effectively make this change permanent and keep their neighboring recipient cells locked in this stem like state. If the number of senescent cells are reduced to that of relatively young tissues (with fewer senescent cells) then a transient SASP response, causing temporary cell reprogramming and subsequent proliferation/differentiation responses would be able to replenish damaged and lost cells.

In some embodiments the Senolytic Agents is applied in Treatment Regimes for tissue rejuvenation therapy of a scalp tissue or an osteoarthritic joint or a pulmonary tissue or a renal tissue. In some embodiments senolytic Agents is administered in Treatment Regimes for scalp treatment and hair regeneration by applying the senolytic tissue externally.

In some embodiments the method of delivery of senolytic peptides in treatment of senescence-associated diseases and disorders are detailed herein this document.

In some embodiments the methods of use of Senolytic Agents for tissue rejuvenation comprise Senolytic Therapy as disclosed in the subject matter of this patent. Some examples of Treatment Regimes of Senolytic Therapy for tissue rejuvenation include administering therapeutically Effective Dose via Impulse Regime, Sustained Regime, and Gentle Regime or via any of the regimes disclosed in Schedule 1. In various embodiments of the Senolytic Therapy for treatment for tissue rejuvenation certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) for Treatment of Senescence-Associated Dermatological Disease or Disorders

Senescence-associated diseases or disorders treatable by administering the Senolytic Agents include dermatological diseases or disorders. Several dermatological diseases or disorder are associated with the accumulation of senescent cells. In some embodiments, said dermatological diseases and disorders are psoriasis, vitiligo and eczema. Among them, vitiligo is an acquired disorder characterized by depigmentation. In addition to genetic susceptibility and autoimmunity, oxidative stress and underlying premature melanocyte senescence are considered to be key factors in vitiligo progression. Melanocytes from non-lesion vitiligo lesions were shown to exhibit a pre-senescent phenotype in vitro.

In some embodiments the methods of use of Senolytic Agents for treatment of dermatological diseases and disorders comprise Senolytic Therapy as disclosed in the subject matter of this patent. Some examples of Treatment Regimes of Senolytic Agents for treatment of dermatological diseases and disorders include administering therapeutically Effective Dose via Impulse Regime, Sustained Regime, and Gentle Regime or via any of the regimes disclosed in Schedule 1. In various embodiments of the Senolytic Therapy for treatment of dermatological diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

In certain appropriate senescence-associated dermatological diseases and disorders the method of application of the senolytic peptides could be external on the surface of the body such that skin cell penetration of said peptides is achieved as described in detail in the disclosed subject matter.

Method Use of the Senolytic Peptide(s) for Treatment of Inflammatory and Autoimmune Diseases and Disorders

Chronic inflammation is a major factor in wide range of disease associated with old age. Recently a global clinical trial of 10,000 subjects who had previous heart attacks of an anti-inflammatory drug which targeted a portion of the inflammatory pathway—focusing specifically on interleukin-1beta (IL-1β), a cell-signaling protein—showed that it reduced their risk of further heart attacks or strokes. The drug prevents these cells from going into overdrive, but presumably leaves the remaining immune system intact. The chronic inflammatory diseases include rheumatoid arthritis when inflammation occurs in joints. Individuals with Rheumatoid arthritis exhibit accelerated immunosenescence possibly as a result of inflammatory mechanisms. Therefore, the Senolytic Agents are suitable for curing or managing chronic inflammation by substantially stopping SASP by killing the senescent cells.

The senescence-associated secretory phenotype (SASP) comprises a range of different proteins, including several proteins known to play a role in aging and age-related diseases, including chemokines such as CCL2 and CLL11 and prominent interleukins such as IL-1, IL-6 and IL-12. When above a certain threshold, such factors can significantly impair tissue function and impair functioning of neighboring cells. As such, senescent cells are thought to be major contributors of inflammation; a theory stating that low, but chronic, levels of inflammation are drivers of age-related decline.

The definition of chronic inflammatory diseases includes osteoarthritis which is characterized by progressive tissue remodeling and loss of joint function and paralleled by increased age. It is the most prevalent disease of the synovial joints. During osteoarthritis, levels of various senescence markers increase in chondrocytes with SASP profiles similar to classical senescent cells which in turn supports the hypothesis that senescence of cells within joint tissues may play a pathological role in the causation of osteoarthritis. Therefore, the Senolytic Agents is suitable to cure or manage chronic inflammation by substantially stopping SASP by killing the senescent cells.

In some embodiments, administration of Senolytic Therapy is effective in the treatment of osteoarthritis. During the senolytic treatment, the osteoarthritis disease parameters which are measured include, inter alia, joint pain, redness, stiffness and/or swelling and joint motion range, X-RAY and/or MRI for bone spurs, blood tests and joint fluid analyses to rule out other causes.

In some embodiments, administration of Senolytic Therapy is effective in the treatment of kyphosis. During the senolytic treatment, the kyphosis disease parameters which are measured include, inter alia, measurement of spine curvature by X-RAY, CT and/or MRI.

In some embodiments the methods of use of Senolytic Agents for treatment of inflammatory or autoimmune diseases and disorders comprise Senolytic Therapy as disclosed in the subject matter of this patent. Some examples of Treatment Regimes of Senolytic Therapy for treatment of inflammatory or autoimmune diseases and disorder include administering therapeutically Effective Dose via Impulse Regime, Sustained Regime, and Gentle Regime or via any of the regimes disclosed in Schedule 1. In various embodiments of the Senolytic Therapy for treatment of inflammatory or autoimmune diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) for Treatment of Cardiovascular Diseases and Disorders

In one embodiment, the senescence-associated disease or disorder treated by the methods described herein is a cardiovascular disease. The cardiovascular disease is often caused by atherosclerosis and is the primary cause of mortality in the developed countries.

There is a growing body of evidence for inflammation as the key process in atherosclerosis into clinical and public health practice. Atherosclerosis is a disease of major arteries in which high levels of low-density lipoprotein bearing oxidative modifications accumulate in vessel walls, attracting phagocytic immune cells to form plaques. Telomere shortening and oxidative stress caused by smooth-muscle proliferation and declining levels of endothelial nitric oxide synthase during plaque formation and expansion cause senescence induction. Human and mouse atheromas have been reported to exhibit senescent vascular smooth muscle and endothelial cells. Basic science and epidemiological studies have developed an impressive case that atherogenesis is essentially an inflammatory response to a variety of risk factors and the consequences of this response lead to the development of acute coronary syndrome. These findings raise the possibility of multi-step involvement of senescent cells in atherogenesis. Therefore, in some embodiments, the Senolytic Agents, administered as Treatment Regimes are suitable to slow down the progression of cardiovascular disease by reducing the chronic inflammation in the body substantially by stopping SASP.

During the Senolytic Therapy of (cardio)vascular disease, (cardio)vascular disease parameters which are measured include, inter alia, cardiac ejection fraction, blood vessel stiffness and blood pressure.

Senolytic Therapy is effective in treatment of atherosclerosis. During the Senolytic Peptide treatment atherosclerosis disease parameters which are measured include blood tests including measurements of cholesterol, glucose, electrocardiogram, angiography, computerized tomography scan and/or ophthalmoscopy.

In some embodiments the methods of use of Senolytic Agents for treatment of cardiovascular diseases and disorders comprise Senolytic Therapy as disclosed in the subject matter of this patent. Some examples of Treatment Regimes of Senolytic Therapy for treatment of cardiovascular diseases and disorders include administering therapeutically Effective Dose via Impulse Regime, Sustained Regime, and Gentle Regime or via any of the regimes disclosed in Schedule 1. In various embodiments of the Senolytic Therapy for treatment of cardiovascular diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

The effectiveness of one or more Senolytic Peptides for treating or preventing (i.e., reducing or decreasing the likelihood of developing or occurrence of) a cardiovascular disease (e.g., atherosclerosis) can readily be determined by a person skilled in the medical and clinical arts. Health status of the subject may be monitored by one or any combination of diagnostic methods, including but not limited to physical examination, assessment and monitoring of clinical symptoms, and performance of analytical tests and methods described herein and practiced in the art (e.g., angiography, electrocardiography, stress test, non-stress test). The effects of the treatment of the Senolytic Peptide can be analyzed using techniques known in the art, such as comparing symptoms of subjects suffering from or at risk of cardiovascular disease that have received the treatment with those of subjects without such a treatment or with placebo treatment.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) for Treatment of Pulmonary Diseases and Disorders

In one embodiment, the senescence-associated disease or disorder treated by the Senolytic Therapy methods described herein is a Pulmonary Diseases and Disorders.

In some embodiments, Pulmonary Disease and Disorders treated by administration of Senolytic Therapy described herein include Chronic obstructive pulmonary disease (COPD)/emphysema characterized by lung inflammation induced by accelerated lung aging involving inflammatory mediators such as tumor necrosis factor alpha, interleukin-1, interleukin-6, reactive oxygen species and proteases. Mechanisms involved in COPD include telomere shortening, cellular senescence, activation of PI3 kinase-mTOR signaling, impaired autophagy, mitochondrial dysfunction, stem cell exhaustion, epigenetic changes, abnormal microRNA profiles, immunosenescence, and a low-grade chronic inflammation. Therefore, the Senolytic Peptide(s) is suitable for treatment of premature senescence involved in Pulmonary Diseases or Disorders by removing the senescent cells in controlled and safe manner.

In some embodiments, Pulmonary Disease and Disorders treated by Senolytic Therapy described herein include Idiopathic Pulmonary Fibrosis (IPF). IPF is the most common and severe idiopathic interstitial pneumonia. In familial interstitial pneumonia, the telomerase complex is affected by the genetic lesions present eventually leading to telomere shortening in both leukocytes and pulmonary tissue which is also observed in sporadic IPF. Pathology of IPF points out to a mechanism with the involvement of cellular senescence in disease progression.

In both COPD and IPF, premature cellular senescence likely affects distinct progenitor's cells (mesenchymal stem cells in COPD, alveolar epithelial precursors in IPF), leading to stem cell exhaustion.

In one embodiment, Senolytic Therapy provided herein is suitable for treating or preventing (i.e., reducing the likelihood of occurrence of pulmonary disease or disorder by killing senescent cells associated with the disease or disorder) particularly the senescence of pulmonary artery-smooth muscle cells in a subject who has the disease or disorder.

In some embodiments, the Senolytic Therapy is effective in the treatment of lung emphysema.

During such a Senolytic Therapy of Pulmonary Diseases and disorders including lung emphysema, the effectiveness of the therapy can be established by taking certain measurements which include but not limited to, inter alia, breathlessness, chest size, lung volume, decreased breath sounds through the stethoscope, fingertip shape, style of breathing, hypoxemia, hypercaria, cyanosis, malnutrition lung volume, lung ejection capacity, dead volume in the lungs, airflow changes after bronchodilator medication, chest X-RAY and CT scan of the chest and red blood cell counts.

In some embodiments, the Senolytic Therapy is effective in the treatment of COPD. During the senolytic therapy of the COPD disease parameters which are measured include, inter alia, spirometry and lung functional tests as described for lung emphysema, including breathlessness, chest size, decreased breath sounds through the stethoscope, fingertip shape, style of breathing, hypoxemia, hypercaria, cyanosis, malnutrition, lung volume, lung ejection capacity, dead volume in the lungs, airflow changes after bronchodilator medication, chest X-RAY and CT scan of the chest and red blood cell counts.

In some embodiments the methods of use of Senolytic Agents for treatment of Pulmonary Disease and disorders comprise Senolytic Therapy as disclosed in the subject matter of this patent. Some examples of Treatment Regimes of Senolytic Therapy for treatment of Pulmonary Disease and disorders include administering therapeutically Effective Dose via Impulse Regime, Sustained Regime, and Gentle Regime or via any of the regimes disclosed in Schedule 1. In various embodiments of the Senolytic Therapy for treatment of Pulmonary Diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) for Treatment of Neurological Diseases and Disorders

Chronic inflammation is a major contributor to a range of neurodegeneration the progressive dysfunction and loss of neurons in the central nervous system whereas neurodegeneration is the major cause of cognitive and motor dysfunction including in Alzheimer's and Parkinson's diseases, neurotropic infections, traumatic brain and spinal cord injury, stroke, neoplastic disorders, prion diseases, multiple sclerosis and amyotrophic lateral sclerosis. Therefore, the Senolytic Peptide(s) is suitable to slow down the progression of neurodegeneration by reducing the chronic inflammation in the body substantially by stopping SASP.

In some embodiments the method of use of Senolytic Agents for senescence-associated neurological diseases or disorders, including but not limited to Parkinson's disease, Alzheimer's disease (and other dementias), motor neuron dysfunction (MND), mild cognitive impairment (MCI), Huntington's disease, and diseases and disorders of the eyes, such as age-related macular degeneration, comprises Treatment Regimes as disclosed in the subject matter of this patent.

In some embodiments the methods of use of Senolytic Agents for treatment of senescence-associated neurological diseases or disorders comprise Senolytic Therapy as disclosed in the subject matter of this patent. Some examples of Treatment Regimes of Senolytic Therapy for treatment of senescence-associated neurological diseases or disorders include administering therapeutically Effective Dose via Impulse Regime, Sustained Regime, and Gentle Regime or via any of the regimes disclosed in Schedule 1. In various embodiments of the Senolytic Therapy for treatment of senescence-associated neurological diseases or disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

In some embodiments the effectiveness of Senolytic Therapy on senescence-associated neurological diseases or disorders, as administered in Treatment Regimes described herein, can be established by monitoring of a subject by a person skilled in the medical and clinical arts. In some embodiments one or any combination of diagnostic methods, including physical examination, assessment and monitoring of clinical symptoms, and performance of analytical tests and methods described herein are used for monitoring the effectiveness of said treatment. The effects of administering one or more Senolytic Peptides can be further analyzed using techniques known in the art, such as comparing symptoms of subjects suffering from or at risk of Alzheimer's disease that have received the treatment with those of subjects without such a treatment or with placebo treatment.

In some embodiments, the Senolytic Agent(s) based Senolytic Therapy is effective in the effective in the treatment of Alzheimer's disease. During the senolytic therapy the Alzheimer's disease parameters which are measured include, inter alia, changes in ability to carry out daily activities, and changes in behavior and personality, tests of memory, problem solving, attention, counting, and language, blood and urine tests, brain scans, such as computed tomography (CT), magnetic resonance imaging (MRI), or positron emission tomography (PET) and/or biomarker analysis.

In some embodiments, the Senolytic Therapy is effective in the treatment of Parkinson's disease. During the senolytic therapy the Parkinson's disease parameters which are measured include, inter alia, analysis for tremors, limb or neck stiffness, general fitness and balance and/or locomotor function.

In some embodiments, the Senolytic Agent(s) based Senolytic Therapy is effective in the treatment of depression. Depression parameters that are to be measured are, inter alia, physical examination, sadness or depressed mood most of the day, major changes in weight, insomnia or excessive sleep, fatigue or loss of energy most of the day, feelings of hopelessness or worthlessness or excessive guilt, problems with concentration or decision making, recurring thoughts of death or suicide.

Method Use of the Senolytic Peptide(s) for Treatment of Ophthalmic Diseases and Disorders

Senescence-associated diseases or disorders treatable by administering the Senolytic Peptide as described in Treatment Regimes herein include ophthalmic diseases or disorders. Such ophthalmic diseases and disorders include but not limited to age-related macular degeneration, cataracts, glaucoma, vision loss, presbyopia. In some embodiments, Ophthalmic Diseases or Disorders involve age related macular degeneration (AMD) resulting in irreversible blindness which is associated with the degradation of retinal pigment epithelium (RPE) cells, photoreceptors, and choriocapillaris. Oxidative stress, inflammation (IL-17 involvement) and some genetic factors are known to be involved in AMD pathogenesis. Oxidative stress can induce DNA damage response (DDR), autophagy, and cell senescence.

Therefore, in some embodiments, the Senolytic Peptide(s) is suitable for treatment of premature senescence involved Ophthalmic Diseases and Disorders by removing the senescent cells in controlled and safe manner.

In some embodiments, Treatment Regimes provided herein for treating or preventing (i.e., reducing the likelihood of occurrence of; delaying the onset or development of, or inhibiting, retarding, slowing, or impeding progression or severity of) an ophthalmic disease, disorder, or condition (e.g., presbyopia, cataracts, macular degeneration); for selectively killing senescent cells in an eye of a subject, and/or inducing collagen production in the eye of a subject in need thereof by administering at least one Senolytic Peptide which may be combined with at least one therapeutically acceptable excipient to form a composition comprising the Senolytic Peptide(s)) directly to an eye.

In some embodiments the methods of use of Senolytic Agents for treatment of ophthalmic diseases and disorders comprise Senolytic Therapy as disclosed in the subject matter of this patent. Some examples of said Treatment Regimes of Senolytic Agents for treatment of ophthalmic diseases and disorders include administering therapeutically Effective Dose via Impulse Regime, Sustained Regime, and Gentle Regime or via any of the regimes disclosed in Schedule 1. In various embodiments of the Senolytic Therapy for treatment of ophthalmic diseases certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

Method Use of the Senolytic Peptide(s) for Treatment of Metabolic Diseases or Disorders

Senescence-associated diseases or disorders treatable or preventable by administering the Senolytic Therapy include metabolic diseases or disorders. Said senescence-associated diseases and disorders include diabetes, metabolic syndrome, diabetic ulcers, and obesity.

Chronic inflammation affects the whole body and is linked to diseases like type 2 diabetes. Low-grade chronic inflammation appears to change the way glucose is absorbed by cells. Anakinra, a biologic anti-inflammatory drug, has been found to improve some diabetes symptoms by blocking the cytokine protein IL-1, as stated above the key instigator of the immune and inflammatory response. Therefore, the Senolytic Therapy is suitable to slow down the progression of the diabetes by reducing the chronic inflammation in the body substantially by stopping SASP.

Diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or by taking measurements which include, inter alia, basal blood glucose levels, average blood glucose levels over a period of time (2-3 months; A1C test), fasting plasma glucose, oral glucose tolerance test, plasma glucose test N.B. physical examination of a subject.

The effectiveness of the Senolytic Therapy can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. In some embodiments a subject who is receiving Senolytic Therapy for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic β-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass Index (BMI) is a measurement tool used to determine excess body weight, and is calculated from the height and weight of a subject. A human is considered overweight when the person has a BMI of 25-29; a person is considered obese when the person has a BMI of 30-39, and a person is considered severely obese when the person has a BMI of 40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorptiometry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly, on the lower extremities. Patients with diabetic venous ulcer exhibit elevated presence of cellular senescence at sites of chronic wounds. Chronic inflammation is also observed at sites of chronic wounds, such as diabetic ulcers suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more of cardiovascular disease, liver steatosis, hyperlipidemia, diabetes, and insulin resistance. A subject with metabolic syndrome may present with a cluster of metabolic disorders or abnormalities which may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

In some embodiments, the diabetes type II disease is effectively treatable or preventable by administering the Senolytic Therapy. During the Senolytic Therapy of diabetes type II disease, the efficacy of the Treatment Regimes is established by taking measurements which-include, inter alia, basal blood glucose levels, average blood glucose levels over a period of time (2-3 months; A1C test), fasting plasma glucose, oral glucose tolerance test, plasma glucose test N.B.

In some embodiments, the obesity disorder is effectively treatable or preventable by administering the Senolytic Therapy. During the Senolytic Therapy the obesity parameters which are measured include alia body weight, Body-Mass-Index (BMI), waist circumference, waist-to-hip ratio, skinfold thicknesses, and bioelectrical impedance, magnetic resonance imaging and/or dual energy X-ray absorptiometry.

In some embodiments, the metabolic syndrome is treatable or preventable effectively by administering the Senolytic Therapy. During the Senolytic Therapy the metabolic syndrome disease parameters which are measured include, inter alia, measurements for obesity (see above, e.g. waist circumference), blood levels of triglycerides, HDL cholesterol, blood pressure, fasting glucose.

The hepatic insufficiency is treatable or preventable effectively by administering the Senolytic Therapy. During the senolytic therapy, the hepatic insufficiency disease parameters which are measured include, inter alia, blood AST and ALT values.

In some embodiments, cirrhosis disease is treatable or preventable effectively by administering the Senolytic Therapy. During the senolytic therapy, the cirrhosis disease parameters which are measured include, inter alia, measurements of blood-clotting factors and international normalized ratio for blood clotting, liver stiffness by magnetic resonance elastography, liver imaging by CT and/or MRI, physical examination, blood testing for bilirubin and creatinine, and/or liver biopsy analysis for liver damage.

164. In some embodiments the methods of use of Senolytic Agents for treatment or prevention of metabolic diseases and disorders comprise Senolytic Therapy as disclosed in the subject matter of this patent. Some examples of Treatment Regimes of Senolytic Therapy for treatment or prevention of metabolic diseases and disorders include administering therapeutically Effective Dose via Impulse Regime, Sustained Regime, and Gentle Regime or via any of the regimes disclosed in Schedule 1. In various embodiments of the Senolytic Therapy treatment or prevention of metabolic diseases and disorders certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) for Treatment of Renal Dysfunction:

Nephrological pathologies, such as glomerular disease, arise in the elderly.

Glomerulonephritis is characterized by inflammation of the kidney and by the expression of two proteins, IL1α and IL1β. IL1α and IL1β are considered master regulators of SASP. Glomerular disease is associated with elevated presence of senescent cells, especially in fibrotic kidneys. Therefore, the Senolytic Peptide(s) is suitable to renal dysfunction by substantially stopping SASP by killing the senescent cells.

In some embodiments, renal insufficiency is effectively treatable or preventable by administering the Senolytic Therapy. During the Senolytic Therapy, the renal insufficiency disease parameters which are measured include, inter alia, blood pressure, heart/lung sound analysis, nervous system exam, urinalysis for protein content, analysis for creatinine clearance and level of Blood Urea Nitrogen, CT, MRI and/or ultrasound of abdomen and kidneys, kidney biopsy for damage analysis.

Glomerulosclerosis is another pathology associated with renal aging supported by the accumulation of senescent cells as indicated by an increase in the levels of senescence markers such as p16 and SA-β-Gal by aging.

In some embodiments, renal dysfunction caused by nephrological pathologies, such as glomerulosclerosis is effectively treatable or preventable by administering the Senolytic Therapy. During the Senolytic Therapy, the glomerulosclerosis disease parameters which are measured include, inter alia, swellings in limbs, weight gains, changes in urine due to proteinuria, distortion or compression of the small capillaries in the glomerulus that filter blood in a biopsy and plasma Urea or protein concentration, blood pressure, glomerular filtration rate, and/or kidney ultrasound.

In some embodiments the methods of use of Senolytic Agents for treatment of renal dysfunction and disorders comprise Senolytic Therapy as disclosed in the subject matter of this patent. Some examples of Treatment Regimes of Senolytic Therapy for treatment of renal dysfunction and disorders include administering therapeutically Effective Dose via Impulse Regime, Sustained Regime, and Gentle Regime or via any of the regimes disclosed in Schedule 1. In various embodiments of the Senolytic Therapy for treatment of renal dysfunction and disorders certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline.

Method Use of the Senolytic Peptide(s) as an Adjuvant Agent in a Cancer Therapy and for Preventing Metastasis

Stress-induced premature senescence (SIPS) occurs rapidly in response to various stresses such as chemotherapeutic drugs and ionizing radiation. Both stresses cause substantial collateral macromolecular damage to non-neoplastic cells and responsible for the early aging phenotypes frequently observed in cancer survivors. Contrary to chronic senescence resulting from normal aging mechanisms and declining macromolecular repair mechanisms, therapy-induced senescence results from abrupt exogenous stresses placed on tissues during cancer therapy. The Senolytic Therapy may be administered to the subjects who may also have cancer, as an adjuvant for prevention, cure, inhibition or retarding the progression of cancer and/or metastasis using the methods as described herein. Metastasis of a cancer occurs when the cancer cells (e.g., tumor cells) spread beyond the anatomical site of origin and initial colonization to other areas throughout the body of the subject. In some embodiments, Senolytic Therapy is selected the primary therapy for prevention, cure, inhibition or retarding the progression of cancer and/or metastasis. In certain embodiments, Senolytic Therapy as an adjuvant therapy includes, but is not limited to, chemotherapy, radiation therapy, hormone therapy, targeted therapy, or biological therapy or other novel therapies that will be recognized by those skilled in the art.

In some embodiments, Senolytic Therapy as an adjuvant for cancer therapy is administered in combination with chemotherapeutic, immunotherapeutic, radiotherapeutic or biological agent(s) used in primary therapy or following a primary therapy for prevention, cure, inhibition or retarding the progression of cancer and/or metastasis

In some embodiments the methods of use of Senolytic Agents for inhibition or retarding the progression of metastasis in a subject who has a cancer comprise Senolytic Therapy as disclosed in the subject matter of this patent. Some examples of Treatment Regimes of Senolytic Therapy for inhibition or retarding the progression of metastasis include administering therapeutically Effective Dose via Impulse Regime, Sustained Regime, and Gentle Regime or via any of the regimes disclosed in Schedule 1. In various embodiments of the Senolytic Therapy for inhibiting or retarding metastasis of cancer certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment Said alterations of Therapy Regime are subject to the decision of the doctor overseeing the therapy particularly when the subject has other diseases or disorders. Such the Senolytic Peptide when administered in a therapeutically Effective Dose to a subject who has a cancer according to the methods described herein may inhibit tumor proliferation.

In some embodiments the methods of use of Senolytic Agents for targeting circulating tumor cells (CTCs) and/or dormant cells and/or cancer stem cells in a subject who has a cancer comprise Senolytic Therapy as disclosed in the subject matter of this patent. Some examples of Treatment Regimes of Senolytic Therapy for targeting circulating tumor cells (CTCs) include administering therapeutically Effective Dose via Impulse Regime, Sustained Regime, and Gentle Regime or via any of the regimes disclosed in Schedule 1. Some examples of Treatment Regimes of Senolytic Therapy for targeting cancer stem cells include administering therapeutically Effective Dose via Impulse Regime, Sustained Regime, and Gentle Regime or via any of the regimes disclosed in Schedule 1. Some examples of Treatment Regimes of Senolytic Therapy for targeting dormant cells include administering therapeutically Effective Dose via Impulse Regime, Sustained Regime, and Gentle Regime or via any of the regimes disclosed in Schedule 1. In various embodiments of the Senolytic Therapy for targeting circulating tumor cells (CTCs) and/or dormant cells and/or cancer stem cells of cancer certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment Said alterations of Therapy Regime are subject to the decision of the doctor overseeing the therapy particularly when the subject has other diseases or disorders. Such the Senolytic Peptide when administered in a therapeutically Effective Dose to a subject who has a cancer according to the methods described herein may inhibit tumor proliferation.

The methods described herein are also applicable for inhibiting, retarding or slowing progression of metastatic cancer, or targeting circulating tumor cells (CTCs) and/or dormant cells and/or cancer stem cells of any one of the types of tumors described in the medical art. In some embodiments multiple Senolytic Therapies are further customized and applied contemporaneously to the same patient for inhibiting, retarding or slowing progression of cancer tumor and/or metastasis and/or for targeting circulating tumor cells (CTCs) and/or dormant cells and/or cancer stem cells. As a non-limiting example, a Senolytic Agent is injected directly into a cancer tumor for inhibiting, retarding or slowing progression of the tumor and/or targeting circulating tumor cells (CTCs) and/or dormant cells and/or cancer stem cells and contemporaneously same or a separate Senolytic Agent is administered as a Senolytic Therapy via intravenous or intraperitoneal delivery method for inhibiting, retarding or slowing progression of metastasis and/or circulating tumor cells (CTCs) and/or dormant cells and/or cancer stem cells.

In some embodiments, the cancer type is but not limited to metastatic melanoma, resistant breast cancer, radiotherapy resistant glioblastoma, colorectal cancer, or thyroid cancer.

In some embodiments, administration of Senolytic Agents as described in Treatment Regimes, are effective in the treatment of metastatic melanoma or as an adjuvant drug. During the senolytic therapy, the metastatic melanoma disease parameters which are measured include, inter alia, a reduction in tumor size and/or metastasization by applying Treatment Regimes as disclosed in the subject matter of this patent i.e. by administering the Senolytic Agents' therapeutically Effective Dose via Impulse Regime, Sustained Regime, and Gentle Regime or via any of the regimes disclosed in Schedule 1.

In some embodiments the methods of use of Senolytic Agents for the treatment of resistant breast cancer as adjuvants, comprise Senolytic Therapy as disclosed in the subject matter of this patent. Some examples of Treatment Regimes of Senolytic Therapy for treatment of resistant breast cancer as adjuvants, include administering therapeutically Effective Dose via Impulse Regime, Sustained Regime, and Gentle Regime or via any of the regimes disclosed in Schedule 1. In various embodiments of the Senolytic Therapy for treatment of resistant breast cancer as adjuvants, certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

In some embodiments, administration of Senolytic Therapy is effective in the treatment of resistant breast cancer or as an adjuvant drug. During the senolytic therapy, the resistant breast cancer disease parameters which are measured include a reduction in tumor size and/or metastasization.

In some embodiments, Senolytic Therapy is the primary therapy for treatment of the resistant breast cancer which involves one or more chemotherapeutic, immunotherapeutic, radiotherapeutic or biological agent(s).

In certain embodiments, the primary therapy agent used for the treatment of the resistant breast cancer is a chemotherapeutic agent(s). Said chemotherapeutic agent(s) include but are not limited to, Tamoxifen, Femara, Herceptin, Letrozole, Taxol, Soltamox, Epirubicin, Trastuzumab, Leuprolide, Paclitaxel, Ellence, Pharmorubicin PFS, Neratinib, Nerlynx, Ogivri and their derivatives.

In some embodiments, the Senolytic Therapy is directed as an adjuvant therapy for the treatment of the resistant breast cancer, during or following Primary Therapy wherein Senolytic Agent is used in combination with at least one Chemotherapeutic Agent.

In some embodiments, Senolytic Therapy, is effective in treatment of a resistant glioblastoma or as an adjuvant drug. During the senolytic therapy, the resistant glioblastoma disease parameters which are measured include, inter alia, a reduction in tumor size and/or metastasization.

In some embodiments the methods of use of Senolytic Agents for the treatment of resistant glioblastoma as adjuvants comprises Senolytic Therapy as disclosed in the subject matter of this patent. Some examples of Treatment Regimes of Senolytic Therapy for the treatment of resistant glioblastoma as adjuvants include administering therapeutically Effective Dose via Impulse Regime, Sustained Regime, and Gentle Regime or via any of the regimes disclosed in Schedule 1. In various embodiments of the Senolytic Therapy for the treatment of resistant glioblastoma as adjuvants, certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment said alterations of Therapy Regime are subject to the decision of the doctor overseeing the therapy particularly when the subject has other diseases or disorders.

In some embodiments, Senolytic Therapy is used as the primary therapy used for treatment of the resistant glioblastoma and involves one or more chemotherapeutic, immunotherapeutic, radiotherapeutic or biological agent(s). Said chemotherapeutic agent(s) include but are not limited to, Temodar, Avastin, Temozolomide, Matulane, Bevacizumab, BiCNU, Gliadel, Carmustine, Hydroxyurea, Procarbazine, Mvasi and their derivatives.

In some embodiments, the Senolytic Agents are used in adjuvant therapy for the treatment of the resistant glioblastoma, in combination with or following primary therapy with at least one chemotherapeutic agent. Said chemotherapeutic agent(s) could be used alone or in combination with the Senolytic Agents. In some embodiments, administration of Senolytic Therapy is effective in the treatment of colorectal cancer or as an adjuvant drug. During the senolytic therapy, the colorectal cancer disease parameters which are measured include a reduction in tumor size and/or metastasization.

In some embodiments the methods of use of Senolytic Agents for the treatment of colorectal cancer as adjuvants comprises Senolytic Therapy as disclosed in the subject matter of this patent. Some examples of Treatment Regimes of Senolytic Therapy for the treatment of colorectal cancer, wherein Senolytic Agents are used as adjuvants, include administering therapeutically Effective Dose via Impulse Regime, Sustained Regime, and Gentle Regime or via any of the regimes disclosed in Schedule 1. In various embodiments of the Senolytic Therapy for the treatment of colorectal cancer, wherein Senolytic Agents are used as adjuvants, certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment said alterations of Therapy Regime are subject to the decision of the doctor overseeing the therapy particularly when the subject has other diseases or disorders.

In some embodiments, the Senolytic Therapy is used as the primary therapy used for treatment of colorectal cancer which involves one or more chemotherapeutic, immunotherapeutic, radiotherapeutic or biological agent(s). Said chemotherapeutic agent(s) include but are not limited to, Xeloda, Oxaliplatin, Avastin, Fluorouracil, Leucovorin, Capecitabine, Irinotecan, Stivarga, Bevacizumab, Erbitux, Camptosar, Cetuximab, Eloxatin, Vectibix, Zaltrap, Betaseron, Fusilev, Lonsurf, Methotrexate, Panitumumab, Wellcovorin, Regorafenib, Mvasi, Keytruda, Opdivo, Cyramza, Interferon beta-1b, Levoleucovorin, Nivolumab, Ramucirumab, Tipiracil/Trifluridine, Ziv-aflibercept, Pembrolizumab, Ipilimumab, Khapzory, Yervoy and their derivatives.

In some embodiments, the Senolytic Agents are used in adjuvant therapy for the treatment of colorectal cancer, in combination with or following primary therapy with at least one chemotherapeutic agent. Said chemotherapeutic agent(s) could be used alone or in combination with the Senolytic Agents. In some embodiments, administration of Senolytic Therapy is effective in the treatment of a thyroid cancer or as an adjuvant drug. During the senolytic therapy, the thyroid cancer disease parameters which are measured include, inter alia, a reduction in tumor size and/or metastasization.

In some embodiments the methods of use of Senolytic Agents for the treatment of thyroid cancer as adjuvants, comprises Senolytic Therapy as disclosed in the subject matter of this patent. Some examples of Treatment Regimes of Senolytic Therapy for the treatment of thyroid cancer as adjuvants, include administering therapeutically Effective Dose via Impulse Regime, Sustained Regime, and Gentle Regime or via any of the regimes disclosed in Schedule 1. In various embodiments of the Senolytic Therapy for the treatment of thyroid cancer as adjuvants, certain parameters Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

In some embodiments, the primary therapy used for treatment of thyroid cancer could involve one or more chemotherapeutic, immunotherapeutic, radiotherapeutic or biological agent(s). Said chemotherapeutic agents include but are not limited to, Armour Thyroid, Nexavar, Nature-Throid, Thyrogen, Caprelsa, Adriamycin, Cometriq, Thyroid desiccated, Sodium iodide-i-131, Sorafenib, Lenvima, Doxorubicin, Lenvatinib, Lodotope, Cabozantinib, Westhroid, Thyrotropin alpha, Vandetanib, Hicon, NP Thyroid, Dabrafenib, Tafinlar, WP Thyroid, i3odine Max, Mekinist, Trametinib and their derivatives.

In some embodiments, the Senolytic Agents are used in adjuvant therapy for the treatment of thyroid cancer, in combination with or following primary therapy with at least one chemotherapeutic agent. Said chemotherapeutic agent(s) could be used alone or in combination with the Senolytic Agents.

In some embodiments, the method of use of Senolytic Agents as treatment or adjuvants for the inhibition of metastasis of a cancer comprises Treatment Regimes as disclosed in the subject matter of this patent. Some examples of said Treatment Regimes include administering therapeutically Effective Dose via Impulse Regime, Sustained Regime, and Gentle Regime or via any of the regimes disclosed in Schedule 1. In various embodiments the Treatment Regimes for therapeutically Effective Dose and/or the duration of cancer treatment stated regimes could be altered depending on the subject's state of health and/or the subject's response to the treatment including but not limited to alteration of therapeutically Effective Dose and/or the duration of treatment, or combinations of Senolytic agents and delivery methods or combinations thereof.

In some embodiments, during the Senolytic Therapy, a subject's progress can be monitored by appropriate measurements including those presented herein or by detecting the senescent cell population on a biopsy sample taken from the subject at the beginning and throughout the period of the therapy and thus monitoring the senescent cell population decline

In other embodiments, during the Senolytic Therapy, a subject's progress could additionally be followed by detecting and/or monitoring metastatic cells and/or cancer cells present in the subject before, during or after the therapy using the methods as recognized by those skilled in the art.

Method of Use of the Senolytic Peptide(s) for Treatment of Progeroid Syndromes

Progeroid syndromes (PSs) are a group of fatal, severe and rare genetic disorders which mimic premature aging while exhibiting various clinical features and phenotypes. PSs mimic many of the characteristics of human ageing such as hair loss, short stature, skin tightness, cardiovascular diseases and osteoporosis. Therefore, the Senolytic Peptides may also be useful for treating or alleviation of the effects of Progeroid Syndromes that occur as a result of premature aging process induced by congenital genetic mutations in individuals. Although all progeroid syndromes are characterized by similar clinical features, their underlying mechanisms can vary depending on the mutated gene and the pathway that is consequently altered. As a result of genomic instabilities due to the mutated genes, premature senescence emerges as a key factor underlying these conditions.

In some embodiments, these syndromes include clinically and genetically heterogeneous diseases such as ataxia-telangiectasia, Bloom syndrome, Cockayne syndrome, Fanconi anaemia, Hutchinson-Gilford Progeria syndrome, Rothmund-Thomson syndrome, trichothiodystrophy, xeroderma pigmentosum, and Werner syndrome (aka adult progeria).

In some embodiments, Progeroid Syndromes include Hutchinson-Gilford Progeria Syndrome (HGPS) which is a rare, fatal and genetic condition of childhood, characterized by growth reduction, failure to thrive, a typical facial appearance (prominent forehead, protuberant eyes, thin nose with a beaked tip, thin lips, micrognathia and protruding ears) and distinct dermatologic features (generalized alopecia, aged-looking skin, sclerotic and dimpled skin over the abdomen and extremities, prominent cutaneous vasculature, dyspigmentation, nail hypoplasia and loss of subcutaneous fat). Individuals with HGPS exhibit atherosclerosis, lipodystrophy, heart infarction and death during puberty.

During the senolytic treatment HGPS disease parameters are measured by, inter alia, features of accelerated aging, hair loss (alopecia), aged-looking skin, joint abnormalities, and a loss of fat under the skin.

In some embodiments, Progeroid Syndromes include Trichothiodystrophy characterized by brittle hair causing hair loss, neurological defects, bone abnormalities and fitness decline.

In other embodiments, PSs include Werner Syndrome characterized by the dramatic, rapid appearance of features associated with normal aging in affected individuals. Affected individuals usually develop accompanying disorders of aging early in life, such as cataracts, skin ulcers, type 2 diabetes, diminished fertility, atherosclerosis, osteoporosis, and some types of cancer.

In some embodiments, premature aging-associated decline and symptoms may be treated or prevented (i.e., the likelihood of occurrence of is reduced) by administering the Senolytic Therapy

In some embodiments the methods of use of Senolytic Agents for treatment of progeroid syndromes comprise Senolytic Therapy as disclosed in the subject matter of this patent. Some examples of Treatment Regimes of Senolytic Therapy for the treatment of progeroid syndromes include administering therapeutically Effective Dose via Impulse Regime, Sustained Regime, and Gentle Regime or via any of the regimes disclosed in Schedule 1. In various embodiments of the Senolytic Therapy for treatment of progeroid syndromes certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders.

The effectiveness of a method of treatment described herein may be manifested by reducing the number of symptoms of a premature aging disease or progeroid trait associated with a senescence-inducing stimulus, decreasing the severity of one or more symptoms, or delaying the progression of a premature aging disease or progeroid trait associated with a senescence-inducing stimulus. In some embodiments, preventing a premature aging disease or progeroid trait associated with a senescence-inducing stimulus refers to preventing (i.e., reducing the likelihood of occurrence) or delaying onset of a premature aging disease or progeroid trait associated with a senescence-inducing stimulus, or reoccurrence of one or more premature aging disease or progeroid trait associated with a senescence-inducing stimulus.

Method of Use of the Senolytic Peptide(s) to Reduce the Side Effects of Chemotherapy and Radiotherapy

Tumor proliferation may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells. It has been demonstrated that a senolytic agent lowers the threshold for senescent cells to enter apoptosis after DNA damage. In some embodiments the Senolytic Agent is used against chemotoxicity or radiation damage by removing the senescent cells in controlled and safe manner.

Because cells may be induced to senesce by cancer therapies, such as radiation and certain chemotherapy drugs (e.g., doxorubicin; paclitaxel; gemcitabine; pomalidomide; lenalidomide), the Senolytic Peptide(s) described herein may be administered after the chemotherapy or radiotherapy to kill (or facilitate killing) of these senescent cells.

As discussed herein and understood in the art, establishment of senescence, such as shown by the presence of a senescence-associated secretory phenotype (SASP), occurs over several days; therefore, administering the Senolytic Peptide to kill senescent cells and/or circulating tumor cells (CTCs) and/or dormant cells and/or cancer stem cells, and thereby reduce the likelihood of occurrence or reduce the extent of metastasis, is initiated when senescence and/or intravasation and/or extravasation has been established. As discussed herein, the following treatment courses for administration of the Senolytic Peptide may be used in methods described herein for treating or preventing (i.e., reducing the likelihood of occurrence, or reducing the severity) a chemotherapy or radiotherapy side effect. Removal or destruction of senescent cells may ameliorate acute toxicity, including acute toxicity comprising energy imbalance, of a chemotherapy or radiotherapy. Acute toxic side effects include but are not limited to gastrointestinal toxicity (e.g., nausea, vomiting, constipation, anorexia, diarrhea), peripheral neuropathy, fatigue, malaise, low physical activity, hematological toxicity (e.g., anemia), hepatotoxicity, alopecia (hair loss), pain, infection, mucositis, fluid retention, dermatological toxicity (e.g., rashes, dermatitis, hyperpigmentation, urticaria, photosensitivity, nail changes), mouth (e.g., oral mucositis), gum or throat problems, or any toxic side effect caused by a chemotherapy or radiotherapy.

Accordingly, in some embodiments, methods are provided herein for ameliorating (reducing, inhibiting, or preventing occurrence (i.e., reducing the likelihood of occurrence)) acute toxicity or reducing severity of a toxic side effect (i.e., deleterious side effect) of a chemotherapy or radiotherapy or both in a subject who receives the therapy, wherein the method comprises administering to the subject an agent that selectively kills, removes, or destroys or facilitates selective destruction of senescent cells. Administration of the Senolytic Peptide for treating or reducing the likelihood of occurrence, or reducing the severity of a chemotherapy or radiotherapy side effect may be accomplished by the same treatment courses described above for treatment/prevention of metastasis. As described for treating or preventing (i.e., reducing the likelihood of occurrence of) metastasis, the Senolytic Peptide is administered in a therapeutically Effective Dose during the off-chemotherapy or off-radiotherapy time interval or after the chemotherapy or radiotherapy treatment regimen has been completed.

In some embodiments the methods of use of Senolytic Agents aim for reduction of the side effects of chemotherapy and radiotherapy comprise Senolytic Therapy as disclosed in the subject matter of this patent. Some examples of Treatment Regimes of Senolytic Therapy for reduction of the side effects of chemotherapy and radiotherapy include administering therapeutically Effective Dose via Impulse Regime, Sustained Regime, and Gentle Regime or via any of the regimes disclosed in Schedule 1. In various embodiments of the Senolytic Therapy for reduction of the side effects of chemotherapy and radiotherapy certain parameters of the Therapy Regimes including, but not limited to, therapeutically Effective Dose and/or combinations of Senolytic Agents, and/or the duration and number of treatment cycles and/or delivery methods or combinations thereof could be altered depending on the subject's state of health and/or the subject's response to the treatment. Said alterations of Therapy Regime are subject to the decision of the doctor overseeing the therapy particularly when the subject has other diseases or disorders

During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. The number of cycles of a chemotherapy or radiotherapy or the total length of time of a chemotherapy or radiotherapy dose regime can vary depending on the subject's response to the cancer therapy. In some embodiments the senolytic therapy plan timeframe will be adjusted and aligned with said chemotherapy or radiotherapy treatment by a person skilled in the oncology art.

In another specific embodiment, the senescence-associated disease or disorder is a atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, stroke, osteoarthritis, osteoporosis, oral mucositis, inflammatory bowel disease, kyphosis, herniated intervertebral disc, Alzheimer's disease, Parkinson's disease, Huntington's disease, dementia, mild cognitive impairment, motor neuron dysfunction, diabetes, diabetic ulcer, metabolic syndrome, obesity, pulmonary fibrosis, chronic obstructive pulmonary disease, asthma, cystic fibrosis, emphysema, bronchiectasis, age-related loss of pulmonary function, macular degeneration, glaucoma, cataracts, presbyopia, vision loss, renal disease, renal failure, frailty, hearing loss, muscle fatigue, skin conditions, skin wound healing, liver fibrosis, pancreatic fibrosis, kidney fibrosis, oral submucosa fibrosis, sarcopenia, eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; cutaneous lupus, atherosclerosis; osteoarthritis; pulmonary fibrosis; hypertension, or chronic obstructive pulmonary disease. In another specific embodiment, the Senolytic Small Molecule is administered directly to an organ or tissue that comprises the senolytic cells. In another specific embodiment, the Senolytic Small Molecule is used to eliminate organ fibrosis or cancer-associated fibrosis. that comprises the senolytic cells. In another specific embodiment, the senescent cells are senescent preadipocytes, senescent endothelial cells, senescent fibroblasts, senescent neurons, senescent epithelial cells, senescent mesenchymal cells, senescent smooth muscle cells, senescent macrophages, or senescent chondrocytes.

Compositions and Methods of Delivery for the Senolytic Peptide

In some embodiments, compositions comprising a Senolytic Peptide can be formulated in a manner appropriate for the delivery method by using techniques routinely practiced in the art. The composition may be in the form of a solid (e.g., tablet, capsule), semi-solid (e.g., gel), liquid, or gas (aerosol). In some embodiments, the Senolytic Peptide (or a composition comprising same) is administered in a therapeutically Effective Dose as a bolus infusion. In some embodiments when the Senolytic Peptide is delivered in a therapeutically Effective Dose by infusion wherein the Senolytic Peptide is delivered to an organ or tissue comprising senescent cells or cancer tumors as an adjuvant drug to a chemotherapy to be killed in accordance with techniques routinely performed by a person skilled in the medical art.

Pharmaceutically-acceptable excipients are well-known in the pharmaceutical arts. Examples of pharmaceutically-acceptable excipients include sterile saline and phosphate buffered saline at physiological pH. Preservatives, stabilizers, dyes, buffers, and the like may be provided in the composition. In some embodiments, the type of excipient is selected based on the mode of administration, as well as the chemical composition of the active ingredient(s). In other embodiments, the Senolytic Peptide is formulated as a lyophilizate. A composition based on the disclosed subject matter may be lyophilized or otherwise formulated as a lyophilized product using one or more appropriate excipient solutions for solubilizing and/or diluting the agent(s) of the composition upon administration. In some embodiments, the Senolytic Peptide is encapsulated within liposomes using technology known and practiced in the art.

In some embodiments, compositions comprising the Senolytic Peptide is formulated and packaged for delivery for any appropriate manner of administration described in the disclosed subject matter and in the art.

A composition comprising the Senolytic Peptide(s) may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocularly, vaginally, rectally, intrathecally, intracranially, or by some combination thereof. In some embodiments, administration of a dose, as described above, is via intravenous, intraperitoneal, directly into the target tissue or organ, or subcutaneous route. In some embodiments, a delivery method includes drug-coated or permeated stents for which the drug is the Senolytic Peptide. Formulations suitable for such delivery methods are described in greater detail herein.

In some embodiments, the Senolytic Peptide (which may be combined with at least one therapeutically-acceptable excipient to form a composition comprising the Senolytic Peptide(s)) is administered in a therapeutically Effective Dose directly to the target tissue or tumor or organ comprising senescent cells that contribute to manifestation of the disease or disorder. In some embodiments when treating osteoarthritis, at least one Senolytic Peptide is administered in a therapeutically Effective Dose directly to an osteoarthritic joint (i.e., intra-articularly) of a subject in need thereof. In some embodiments, the Senolytic Peptide(s) may be administered in a therapeutically Effective Dose to the joint via topical, transdermal, intradermal, or subcutaneous route. In some embodiments, methods are provided herein for treating a cardiovascular disease or disorder associated with arteriosclerosis, such as atherosclerosis by administering directly into an artery. In some embodiments, the Senolytic Peptide (which may be combined with at least one pharmaceutically-acceptable excipient to form a composition comprising the Senolytic Peptide(s)) for treating a senescence-associated pulmonary disease or disorder may be administered in a therapeutically Effective Dose by inhalation, intranasally, by intubation, or intracheally, for example, to provide the Senolytic Peptide more directly to the affected pulmonary tissue. By way of another non-limiting example, the Senolytic Peptide (or composition comprising the Senolytic Peptide) may be delivered directly to the eye either by injection (e.g., intraocular or intravitreal) or by conjunctival application underneath an eyelid of a cream, ointment, gel, or eye drops. In some embodiments, the Senolytic Peptide or a composition comprising the Senolytic Peptide may be formulated as a timed release (also called sustained release, controlled release) composition or may be administered in a therapeutically Effective Dose as a bolus infusion.

A composition comprising the Senolytic Peptide(s) (e.g., for oral administration or for injection, infusion, subcutaneous delivery, intramuscular delivery, intraperitoneal delivery or other method) may be in the form of a liquid. A liquid composition comprising the Senolytic Peptide(s) may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable composition comprising the Senolytic Peptide(s) is preferably sterile. In another embodiment, for treatment of an ophthalmological condition or disease, a liquid composition comprising the Senolytic Peptide(s) may be applied to the eye in the form of eye drops. A liquid composition comprising the Senolytic Peptide(s) may be delivered orally.

For oral formulations, at least one of the Senolytic Peptides described herein can be used alone or in combination with appropriate additives to make tablets, powders, granules or capsules, and if desired, with diluents, buffering agents, moistening agents, preservatives, coloring agents, and flavoring agents. The compounds may be formulated with a buffering agent to provide for protection of the compound from low pH of the gastric environment and/or an enteric coating. The Senolytic Peptide included in a composition comprising the Senolytic Peptide(s) may be formulated for oral delivery with a flavoring agent, e.g., in a liquid, solid or semi-solid formulation and/or with an enteric coating.

A composition comprising any one of the Senolytic Peptides described herein may be formulated for sustained or slow release (also called timed release or controlled release). Such compositions comprising the Senolytic Peptide may generally be prepared using well known technology and administered in a therapeutically Effective Dose by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active agent contained within a sustained release formulation depends upon the site of implantation, the rate and expected duration of release, and the nature of the condition, disease or disorder to be treated or prevented.

In some embodiments, the compositions comprising the Senolytic Peptide are formulated for transdermal, intradermal, or topical administration. Said compositions can be administered in a therapeutically Effective Dose using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered in a therapeutically Effective Dose topically or injected directly into the skin adjacent to or within the area to be treated (intradermally or subcutaneously). The active compositions comprising the Senolytic Peptide can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

In some embodiments, compositions comprising the Senolytic Peptide can be formulated as emulsions for topical application. An emulsion contains one liquid distributed within the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically-approved excipients. Suitable surfactants include, but are not limited to, anionic surfactants, non-ionic surfactants, cationic surfactants, and amphoteric surfactants. Compositions comprising the Senolytic Peptide for topical application may also include at least one suitable suspending agent, antioxidant, chelating agent, emollient, or humectant.

In some embodiments, ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions comprising the Senolytic Peptide, patches, bandages and articles. These systems are semi solid emulsions, micro-emulsions, or foam emulsion systems.

In some embodiments, the Senolytic Peptide(s) can be formulated with oleaginous bases or ointments to form a semisolid composition with a desired shape. In addition to the Senolytic Peptide, these semisolid compositions comprising the Senolytic Peptide can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

In some embodiments, a composition comprising any one of the Senolytic Peptides described herein may be formulated for sustained or slow release (which may also be called timed release or controlled release). Controlled or sustained release transdermal or topical formulations can be achieved by the addition of time-release additives, such as polymeric structures, matrices, that are available in the art. For example, the compositions comprising the Senolytic Peptide may be administered in a therapeutically Effective Dose through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

In some embodiments, an insert, transdermal patch, bandage or article comprise a mixture or coating of polymers that provide release of the active agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. In some embodiments, rate controlling polymers are useful for administration to sites where pH change can be used to effect release. Said rate controlling polymers are applied using a continuous coating film during the process of spraying and drying with the active compound. In one embodiment, the coating formulation is used to coat pellets comprising the active ingredients that are compressed to form a solid, biodegradable insert.

In some embodiments, a polymer formulation is utilized to provide controlled or sustained release including bioadhesive polymers. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. In some embodiments, said polymer is in the form of a coating or part of a bandage, stent, catheter, vascular graft, or other device to facilitate delivery of the Senolytic Peptide. The matrices can be formed by solvent evaporation, spray drying, solvent extraction and other methods known to those skilled in the art.

In some embodiments of a method described herein for treating a cardiovascular disease associated with or caused by arteriosclerosis, one or more Senolytic Peptide(s) may be delivered directly into a blood vessel (e.g., an artery) via a stent. In some embodiments, a stent is used for delivering the Senolytic Peptide to an atherosclerotic blood vessel (an artery). Several methods are described in the art for preparing drug-coated and drug-embedded stents. In some embodiments the Senolytic Peptide may also be incorporated into the stent (for example as a coating or pores in the metal stent itself). In some embodiments, the Senolytic Peptide may be formulated within liposomes and applied to a stent. Placement of stents in an atherosclerotic artery is performed by a person skilled in the medical art.

In some embodiments, the Senolytic Peptide is administered in a therapeutically Effective Dose to a subject who has an ophthalmic senescence-associated or disease or disorder may be delivered intraocularly or intravitreally. In other some embodiments, the Senolytic Peptide(s) may be administered in a therapeutically Effective Dose to the eye by a conjunctival route, applying the Senolytic Peptide to the mucous membrane and tissues of the eyelid, either upper, lower, or both. Any of these administrations may be bolus infusions.

In some embodiments, the Senolytic Peptide(s) is administered in a therapeutically Effective Dose in the form of PEGylated peptide. PEGylation is an alternative route for some peptides which could not be cyclic. Amphiphilicity of PEG increases the solubility of the said peptides in both organic solvents and water. Direct PEGylation of peptides increase absorption and systemic stability of said peptides. Additionally, PEG and its metabolites are non-toxic at the concentrations that are used for peptide delivery. As will be recognized by those skilled in the art the Senolytic Peptide(s) could be PEGylated. In some embodiments of the Senolytic Peptide(s) is PEGylated.

In some embodiments, the fatty acid conjugated Senolytic Peptide(s) is administered in a therapeutically Effective Dose. Lipidization has been used to increase protein bioavailability during oral administration. Conjugation of the polypeptides with fatty acids improves the transport across membranes and confers the peptide with higher stability and half-life. As will be recognized by those skilled in the art the Senolytic Peptide(s) could be conjugated with fatty acids by lipidization. In some embodiments of the Senolytic Peptide(s) is conjugated with fatty acids by lipidization.

In some embodiments, vitamin B12 conjugated Senolytic Peptide(s) is administered in a therapeutically Effective Dose. Conjugation of peptides to vitamin B12 and its derivatives is a method to increase oral absorption said peptides by exploiting the receptor mediated absorption of vitamin B12 bound to intrinsic factor. As will be recognized by those skilled in the art the Senolytic Peptide(s) could be conjugated with vitamin B12 or its derivatives. In some embodiments of the Senolytic Peptide(s) is conjugated with vitamin B12 or its derivatives.

In some embodiments, stapled Synthetic Peptide(s) is administered in a therapeutically Effective Dose. Stapled peptides have an alpha helical structure wherein various residues are linked by a synthetic hydrocarbon backbone. They have been used in the drug delivery to improve the biochemical properties of the delivered peptides by locking the peptide conformation, increasing the helicity and solution stability of the said peptides. As will be recognized by those skilled in the art the Senolytic Peptide(s) could be synthesized as stapled peptides. In some embodiments of the Senolytic Peptide(s) is synthesized as stapled peptides.

In some embodiments, N or C-terminally modified Synthetic Peptide(s) is administered in a therapeutically Effective Dose. N and/or C terminal modification of peptides can confer stability by increasing resistance to proteolysis. N-acetylation and C-amidation have been exhibited to improve resistance to proteolytic degradation. As will be recognized by those skilled in the art the Senolytic Peptide(s) could be N-acetylated or C-amidated. In some embodiments of the Senolytic Peptide(s) is N-acetylated or C-amidated.

In some embodiments, the Senolytic Peptide(s) is administered in a therapeutically Effective Dose as Prodrugs. Prodrugs are derived from drug molecules. They are bioreversible compounds which can release the active parent drug in vivo through enzymatic or chemical transformation. As will be recognized by those skilled in the art the Senolytic Peptide(s) could be synthesized as Prodrug peptides. In some embodiments of the Senolytic Peptide(s) is synthesized as Prodrug peptides.

As will be recognized by those skilled in the art in some embodiments the Senolytic Peptide(s) could be co-administered with enzyme inhibitors to alleviate proteolytic degradation thereby increasing bioavailability. In some embodiments of the Senolytic Peptide(s) is co-administered with enzyme inhibitor.

As will be recognized by those skilled in the art in some embodiments, the Senolytic Peptide(s) could be co-administered with absorption enhancers such as chitin and its derivatives such as chitosan to enhance absorption of hydrophilic drug molecules. In some embodiments of the Senolytic Peptide(s) is co-administered with absorption enhancers such as chitin and its derivatives (i.e. chitosan).

### EXAMPLES

### Example 1: Senolytic Peptide(s) Selectively Clears Senescent Cells

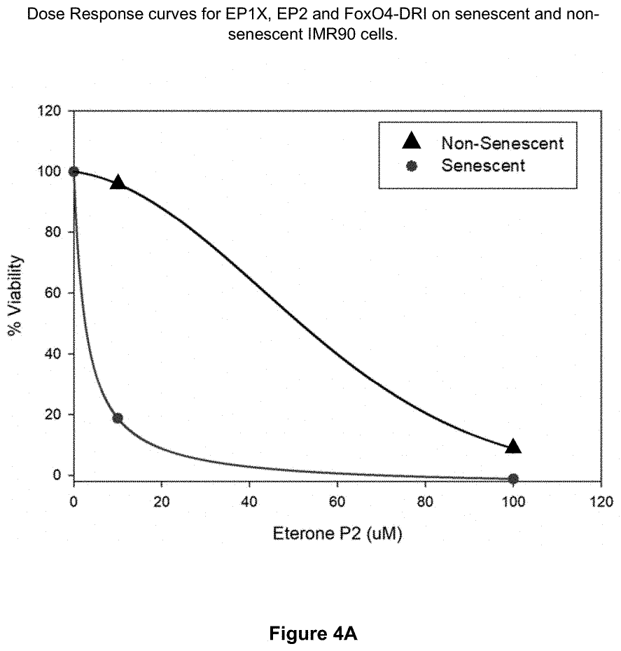

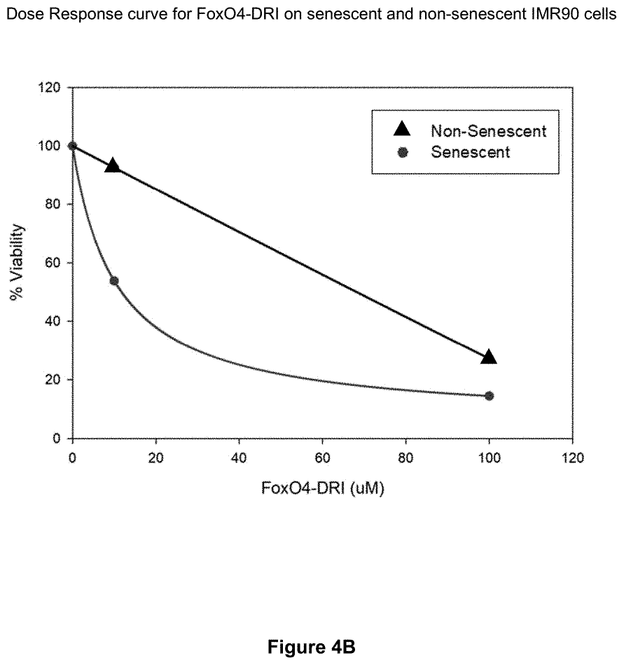

IMR-90 cells (ATCC # CCL-186, a diploid primary human fibroblast adherent cell line derived from fetal lung tissue) were treated with 100 nM Doxorubicin twice every other day for senescence induction. After 5 days, the cells were assayed for Senescece-Associated B-Galactosidase (SA-B-Gal) activity (Senescence Detection Kit, Abcam) and confirmed to possess SA-B-Gal activity. Later these cells were used for experiments comparing them to their non-Doxorubicin (non-senescent) treated counterparts. Senescent and non-senescent IMR90 fibroblasts were plated for XTT viability assays. 7000 senescent (obtained as described above) and 2000 non-senescent IMR90 fibroblasts were plated in 96-well plates incubated. The cells were treated with mock (PBS) or the Senolytic Peptide(s) at total doses ranging from 0 to 100 NM. The mock and peptide treatments were carried out for 3 consecutive days (24, 48 and 72 hours) after plating. After treatment period, the cells were incubated for 7 days until XTT-based viability was analyzed according to the manufacturer's instructions. The senescent cells showed much steeper decrease in viability compared with the non-senescent cells. The Senolytic Peptide(s) were ordered from Chinese Peptide Company with TFA removal. The Senolytic Peptide(s) were identified through virtual screening to interfere with the FoxO4 CR3 domain to liberate p53 from the FoxO4-p53 complex. The Senolytic Peptide (SEQ ID NO: 2) selectively decreased the viability of senescent cells but not non-senescent cells (FIG. 4A). The senolytic effect of the Senolytic Peptide (SEQ ID NO: 2) was superior to FoxO4-DRI as shown by a sharper decrease in viability curves of the senescent IMR90 cells (FIG. 4B).

### Example 2: Chemotherapy (Doxorubicin) Induces Senescence In Vitro which is Counteracted by the Senolytic Peptide(s)

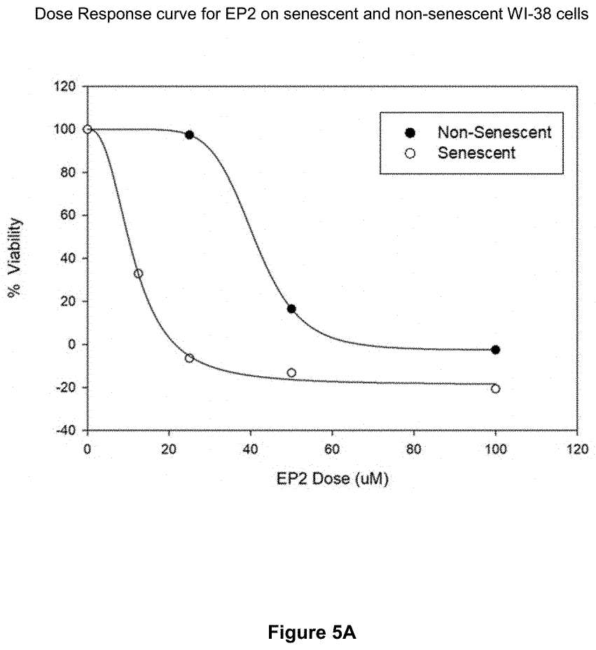

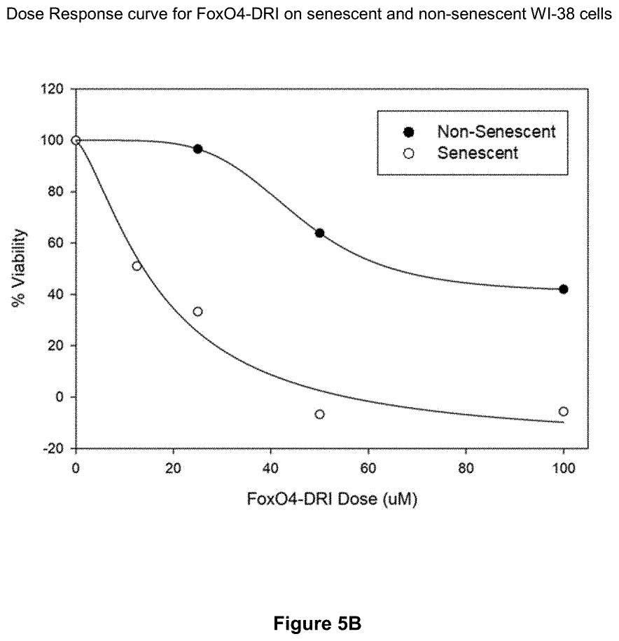

WI-38 cells (ATCC # CCL-75, a diploid primary human fibroblast adherent cell line derived from fetal lung tissue) were treated with a chemotherapy agent, 100 nM Doxorubicin twice every other day to induce senescence. After 5 days, the cells were assayed for Senescence-Associated B-Galactosidase (SA-B-Gal) activity (Senescence Detection Kit, Abcam) and confirmed to possess SA-B-Gal activity. Later these cells were used for experiments comparing them to their non-Doxorubicin (non-senescent) treated counterparts. Senescent and non-senescent WI-38 fibroblasts were plated for XTT viability assays. 7000 senescent (obtained as described above) and 2000 non-senescent WI-38 fibroblasts were plated in 96-well plates incubated. The cells were treated with mock (PBS) or the Senolytic Peptide (SEQ ID NO: 11) at total doses ranging from 0 to 100 μM. The mock and peptide treatments were carried out for 3 consecutive days (24, 48 and 72 hours) after plating. After treatment period, the cells were incubated for 7 days until XTT-based viability was analyzed according to the manufacturer's instructions. The senescent cells showed much steeper decrease in viability compared with the non-senescent cells. The Senolytic Peptide (SEQ ID NO: 2) was ordered from Chinese Peptide Company with TFA removal. Senolytic Peptide (SEQ ID NO: 11) selectively decreased the viability of senescent cells but not non-senescent cells (FIG. 5A). The senolytic effect of the Senolytic Peptide (SEQ ID NO: 2) was superior to FoxO4-DRI as shown by a sharper decrease in viability curves of the senescent WI-38 cells (FIG. 5B)

### Example 4: Senolytic Peptide(s) is Non-Toxic at Therapeutically Effective Doses

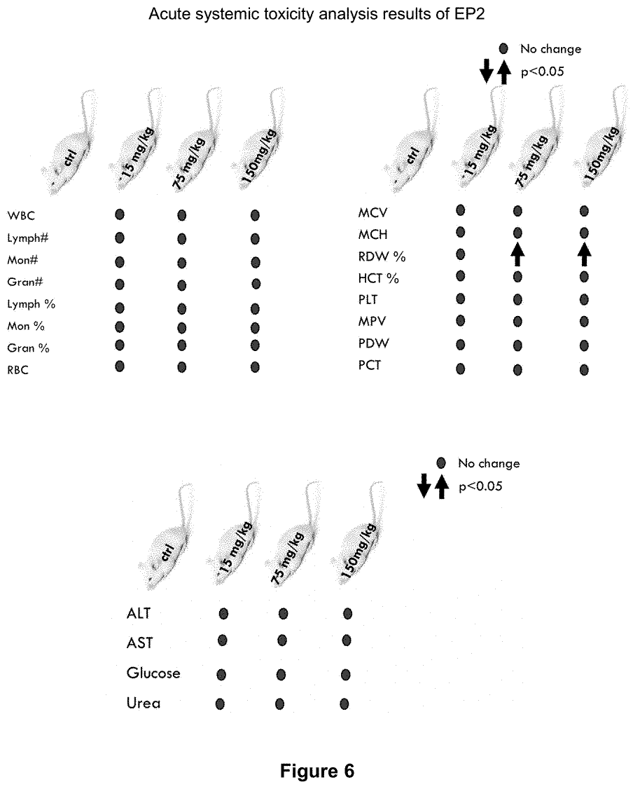

Senolytic Peptides were evaluated for toxicity according to Acute Sytemic Toxicity Test (ISO 10993-11). This study was performed in strict accordance with the protocol approved by the TUBITAK-MAM Animal Ethics Committee. All the mice used in this study were of a BALB/c background at 8-12 weeks of age. All mice were kept in group housing until the start of the experiment after which they were placed in separate cages (5 mice per cage). The Senolytic Peptide (SEQ ID NO: 2) is administered intravenously using a mouse model (BALB/c) at doses of 1, 10, 50 and 100 mg/kg and vehicle alone is also administered using single injections (n=5, per group). Control and Senolytic Peptide (SEQ ID NO: 2) injected mice were sacrificed 24 hours later for pathological examination. No toxicity to the physical appearance of the mice was observed within 24 hours of injection. Blood samples were subsequently collected for determination and analysis of hematological and biochemical parameters. Plasma alanine aminotransferase (ALT), aspartate aminotransferase (AST), alkaline phosphatase (ALP) activities and blood urea, glucose, triglyceride levels were determined, inter alia, full hematological parameters. The results indicated that even at higher doses (150 mg/kg) than doses used in example 3, hematological parameters, AST, ALT, ALP levels and gross pathology results indicate no toxicity to the said mice model (FIG. 6).

The following embodiments demonstrate additional aspects of the disclosed subject matter.

A first embodiment is an artificial peptide comprises an amino acid sequence having at least 90% identity to any one of SEQ ID NO: 1 through SEQ ID NO: 19.

A second embodiment is the artificial peptide according to the first embodiment, wherein the amino acid sequence has at least 95% identity to any one of SEQ ID NO: 1 through SEQ ID NO: 19.

A third embodiment is the artificial peptide according to any one of the first through the second embodiments, wherein the amino acid sequence has at least 98% identity to any one of SEQ ID NO: 1 through SEQ ID NO: 19.

A fourth embodiment is the artificial peptide according to any one of the first through the third embodiments, wherein the amino acid sequence comprises any one of SEQ ID NO: 1 through SEQ ID NO: 19.

A fifth embodiment is the artificial peptide according to any one of the first through the fourth embodiments, wherein the amino acid sequence has at least 70% identity to any one of SEQ ID NO: 1 through SEQ ID NO: 19.

A sixth embodiment is the artificial peptide according to any one of the first through the fifth embodiments, wherein the amino acid sequence has at least 75% identity to any one of SEQ ID NO: 1 through SEQ ID NO: 19.

A seventh embodiment is the artificial peptide according to any one of the first through the sixth embodiments, wherein the amino acid sequence comprises any one of SEQ ID NO: 1 through SEQ ID NO: 19.

An eighth embodiment is the artificial peptide according to any one of the first through the seventh embodiments, wherein the artificial peptide further comprises an N-terminal amino acid sequence that facilitates cellular uptake or a C-terminal amino acid sequence that facilitates cellular uptake.

A ninth embodiment is the artificial peptide according to any one of the first through the eighth embodiments, wherein the artificial peptide further comprises an N-terminus amino acid sequence comprising a single D-amino acid.

A tenth embodiment is the artificial peptide according to any one of the first through the ninth embodiments, wherein the artificial peptide exhibits a circular structure.

An eleventh embodiment is a composition comprising the artificial peptide according to any one of the first through the tenth embodiments.

A twelfth embodiment is a method of inducing the apoptosis of a senescent cell in a subject, the method comprising causing an artificial peptide comprising an amino acid sequence having at least 90% identity to any one of SEQ ID NO: 1 through SEQ ID NO: 19 to interfere with the CR3 domain of Forkhead box protein 04 (FoxO4) of the senescent cell.

A thirteenth embodiment is the method according to the twelfth embodiment, wherein the amino acid sequence has at least 95% identity to any one of SEQ ID NO: 1 through SEQ ID NO: 19.

A fourteenth embodiment is the method according to any one of the twelfth through the thirteenth embodiments, wherein the amino acid sequence has at least 95% identity to any one of SEQ ID NO: 1 through SEQ ID NO: 19.

A fifteenth embodiment is the method according to any one of the twelfth through the fourteenth embodiments, wherein the amino acid sequence comprises any one of SEQ ID NO: 1 through SEQ ID NO: 19.

A sixteenth embodiment is the method according to any one of the twelfth through the fifteenth embodiments, wherein the amino acid sequence has at least 90% identity to any one of SEQ ID NO: 1 through SEQ ID NO: 19.

A seventeenth embodiment is the method according to any one of the twelfth through the sixteenth embodiments, wherein the amino acid sequence has at least 95% identity to any one of SEQ ID NO: 1 through SEQ ID NO: 19.

A eighteenth embodiment is the method according to any one of the twelfth through the seventeenth embodiments, wherein the amino acid sequence comprises any one of SEQ ID NO: 1 through SEQ ID NO: 19.

A nineteenth embodiment is the method according to any one of the twelfth through the eighteenth embodiments, wherein the artificial peptide further comprises an N-terminal amino acid sequence that facilitates cellular uptake or a C-terminal amino acid sequence that facilitates cellular uptake.

A twentieth embodiment is the method according to any one of the twelfth through the nineteenth embodiments, wherein the artificial peptide further comprises an N-terminus amino acid sequence comprising a single D-aa.

A twenty-first embodiment is the method according to any one of the twelfth through the twentieth embodiments, wherein the artificial peptide exhibits a circular structure.

A twenty-second embodiment is the method according to any one of the twelfth through the twenty-first embodiments, wherein causing the artificial peptide to interfere with the CR3 domain of FoxO4 of the senescent cell comprises administering a pharmaceutical composition comprising the artificial peptide according to any one of claims 12-21 to the subject.

A twenty-third embodiment is the method according to any one of the twelfth through the twenty-second embodiments, wherein the artificial peptide exhibits maximized interference with the CR3 domain of FoxO4.

A twenty-fourth embodiment is the method according to any one of the twelfth through the twenty-third embodiments, wherein the artificial peptide exhibits reduced direct interaction with the p53DBD compared to that of the endogenous FoxO4.

A twenty-fifth embodiment is the method according to any one of the twelfth through the twenty-fourth embodiments, wherein the artificial peptide preferably exhibits a reduced interference with the CR3 domains of FoxO1 or FoxO3 compared to that of said FoxO4.

A twenty-sixth embodiment is the method according to any one of the twelfth through the twenty-fifth embodiments, wherein the artificial peptide exhibits reduced interference with DNA compared to that of said FoxO4.

A twenty-seventh embodiment is the method according to any one of the twelfth through the twenty-sixth embodiments, wherein the senescent cell is characterized as expressing the senescence-associated secretory phenotype (SASP).

A twenty-eighth embodiment is the method according to any one of the twelfth through the twenty-seventh embodiments, wherein the method comprises treatment of a senescence-associated disease or disorder.

A twenty-ninth embodiment is the method according the twenty-eighth embodiment, wherein the disease or disorder is cancer, and wherein the subject is a mammal, preferably a human, and wherein the artificial peptide is administered before, during, and/or after subjecting the subject to radiation therapy, and/or before, during or after administering to the subject at least one chemotherapeutic agent.

A thirtieth embodiment is the method according to any one of the twenty-eighth through the twenty-ninth embodiments, wherein the said cancer is characterized as resistant to therapy.

A thirty-first embodiment is the method according to any one of the twenty-eighth through the thirtieth embodiments, wherein said therapy-resistant cancer comprises is metastatic melanoma, breast cancer, or glioblastoma, and wherein the therapy to which the cancer is resistant is radiation therapy or chemotherapy.

A thirty-second embodiment is the method according to any one of the twelfth through the thirty-first embodiments, wherein the subject comprises a human characterized as suffering from, or expected to suffer from chronic inflammatory diseases or a senescence related disease or disorder.

A thirty-third embodiment is the method according to any one of the twelfth through the thirty-second embodiments, wherein the method is effective to remove cells from the subject that express p16INK4a, wherein the subject is characterized as suffering from, or expected to suffer from a senescence-associated disease or disorder.

A thirty-fourth embodiment is the method according to any one of the twelfth through the thirty-third embodiments, wherein the method is effective to alter levels of the Serine-46 phosphorylated p53 foci in the subject, wherein the subject is characterized as suffering from, or expected to suffer from, a senescence-associated disease or disorder.

A thirty-fifth embodiment is the method according to any one of the twelfth through the thirty-fourth embodiments, wherein the method comprises administering the artificial peptide according to an Impulse Regime, Sustained Regime, a Gentle Shock Regime, or combinations thereof.

A thirty-sixth embodiment is a cell penetrating peptide comprising an amino acid sequence having at least 70% identity to any one of SEQ ID NO: 1 through SEQ ID NO: 19.

A thirty-seventh embodiment is the cell penetrating peptide of the thirty-sixth embodiment, wherein the an amino acid sequence has at least 80% identity to any one of SEQ ID NO: 1 through SEQ ID NO: 19.

A thirty-eighth embodiment is the cell penetrating peptide of one of the thirty-sixth through the thirty-seventh embodiments, wherein the amino acid sequence has at least 90% identity to any one of SEQ ID NO: 1 through SEQ ID NO: 19.

A thirty-ninth embodiment is a Senolytic Peptide(s) comprising the amino acid sequence of one of the thirty-sixth through the thirty-eighth embodiments, wherein the artificial peptide further comprises an N-terminal amino acid sequence that facilitates cellular uptake or a C-terminal amino acid sequence that facilitates cellular uptake.

A fortieth embodiment is the cell penetrating peptide of one of the thirty-sixth through thirty-ninth embodiments, wherein the cell penetrating peptide further comprises an N-terminus amino acid sequence comprising a single D-amino acid insertion.

A forty-first embodiment is the cell penetrating peptide of one of the thirty-sixth through thirty-ninth embodiments, wherein the cell penetrating peptide exhibits a circular structure.

A forty-second embodiment is a method for treating a senescence-associated disease or disorder comprising administering to a subject in need thereof a therapeutically-effective amount of a peptide comprising an amino acid sequence having at least 70% identity to any one of SEQ ID NO: 1 through SEQ ID NO: 19 that selectively kill senescent cells over non-senescent cells; wherein said peptide is not used for intracellular delivery vehicle for another compound, wherein said disorder is selected from the group of age-related disorders consisting of atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness, wherein the method of administration of therapeutically-effective amount of said peptide is by any of three shock dose regimes namely Impulse Regime, Sustained Shock Regime, and Gentle Shock Regime, wherein each dose regime independently comprises a treatment course whose duration varies from 1 day to 2 weeks and followed by a non-treatment interval of at least 2 weeks.

A forty-third embodiment is the method of use of the forty-second embodiment, wherein said therapeutically Effective Dose and said method of administration are as described in Schedule 1.

A forty-fourth embodiment is the method of use of one of the forty-second through forty-third embodiments, wherein said disorder is cancer, and wherein the use is for administration to a mammalian subject, preferably a human, before, during and/or after subjecting said subject to radiation therapy, and/or before, during or after administering to said subject at least one chemotherapeutic agent.

A forty-fifth embodiment is the method of use of the forty-fourth embodiment, wherein the cancer is a therapy-resistant cancer.

A forty-sixth embodiment is the method of use of the forty-fifth embodiment, wherein said therapy-resistant cancer is metastatic melanoma, breast cancer or glioblastoma, preferably metastatic melanoma, and wherein said therapy to which said cancer is resistant is radiation therapy or chemotherapy.

A forty-seventh embodiment is the method of use of one of the forty-second through forty-fourth embodiments, wherein said peptide is used for removing senescent cells in a human subject suffering from, or expected to suffer from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

A forty-eighth embodiment is the method of use of one of the forty-second through forty-fifth embodiments, wherein said peptide is used for removing cells that express p16INK4a in a subject suffering from, or expected to suffer from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

A forty-ninth embodiment is the method of use of one of the forty-second through forty-fifth embodiments, wherein said peptide is used for altering the levels of the Serine-46 phosphorylated p53 foci in a subject suffering, or expected to suffer, from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

A fiftieth embodiment is a pharmaceutical peptide composition comprising any amino acid sequence which have 70% sequence resemblance with any one of said peptide of one of the thirty-sixth through forty-fifth embodiments and wherein the fragment length is at least 10 amino acids.

A fifty-first embodiment is a pharmaceutical composition comprising any amino acid sequence which is identical to any one of said peptides of one of the thirty-sixth through forty-fifth embodiments, wherein amino acid sequences are expressed in D-symmetry and/or in a retroreversed sequence and contain non-natural amino acids.

A fifty-second embodiment is a pharmaceutical composition comprising amino acid sequence which is identical to said peptide of one of the thirty-sixth through forty-fifth embodiments, wherein the structure of the listed peptides is in circular form.

A fifty-third embodiment is a pharmaceutical composition comprising any amino acid sequence which is identical to any of of the said peptides of one of the thirty-sixth through forty-fifth embodiments, wherein peptidomimetics are applied to mimic 70% of the claimed sequence.

A fifty-fourth embodiment is a pharmaceutical composition comprising any amino acid sequence which is identical to any one of said peptide of one of the thirty-sixth through forty-fifth embodiments, wherein the CCPs further comprises an N-terminal or C-terminal modification.

A fifty-fifth embodiment is a pharmaceutical composition comprising of one of the thirty-sixth through forty-fifth embodiments wherein nucleic acid encoding of said senolytic CPP is comprised in a viral or nonviral expression vector.

A fifty-sixth embodiment is the method of use of one of the thirty-sixth through forty-fifth embodiments, wherein said peptide is any one of the said pharmaceutical composition.

A fifty-seventh embodiment is a method for treating a senescence-associated disease or disorder comprising administering to a subject in need thereof a therapeutically-effective amount of a pharmaceutical compound which is any one of the senolytic iopromide compounds that selectively kill senescent cells over non-senescent cells; wherein iopromide is not used for image enhancement in radiology wherein said disorder is selected from the group of age-related disorders consisting of atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness, wherein the method of administration of therapeutically-effective amount of said compound is by any of three shock dose regimes namely Impulse Regime, Sustained Shock Regime, and Gentle Shock Regime, wherein each dose regime independently comprises a treatment course whose duration varies from 1 day to 2 weeks and followed by a non-treatment interval of at least 2 weeks.

A fifty-eighth embodiment is the method of use of the fifty-seventh embodiment, wherein said disorder is cancer, and wherein the use is for administration to a mammalian subject, preferably a human, before, during and/or after subjecting said subject to radiation therapy, and/or before, during or after administering to said subject at least one chemotherapeutic agent.

A fifty-ninth embodiment is the method of use of the fifty-eighth embodiment, wherein the said cancer is a cancer resistant to therapy.

A sixtieth embodiment is the method of use of one of the fifty-seventh through fifty-eighth embodiments, wherein said therapy-resistant cancer is metastatic melanoma, breast cancer or glioblastoma, preferably metastatic melanoma, and wherein said therapy to which said cancer is resistant is radiation therapy or chemotherapy.

A sixty-first embodiment is the method of use of one of the fifty-seventh through fifty-eighth embodiments, wherein said compound is used for removing senescent cells in a human subject suffering from, or expected to suffer from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

A sixty-second embodiment is the method of use of one of the fifty-seventh through fifty-eighth embodiments, wherein said compound is used for removing cells that express p16INK4a in a subject suffering from, or expected to suffer from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

A sixty-third embodiment is the method of use of one of the fifty-seventh through sixty-second embodiments, wherein said compound is used for for altering the levels of the Serine-46 phosphorylated p53 foci in a subject suffering, or expected to suffer, from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

As will be recognized by those skilled in the art, the Senolytic Peptide(s) and the disclosed method described in the present application can be modified and varied over a tremendous range of applications to produce a wide range of senolytic peptides which may be directed to specific therapies which target those subsets of senescent cells and accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

What is claimed is:

1. A cell penetrating peptide comprising an amino acid sequence having at least 70% identity to any one of SEQ ID NO: 1 through SEQ ID NO: 19.

2. The cell penetrating peptide of claim 1, wherein the an amino acid sequence has at least 80% identity to any one of SEQ ID NO: 1 through SEQ ID NO: 19.

3. The cell penetrating peptide of claim 2, wherein the amino acid sequence has at least 90% identity to any one of SEQ ID NO: 1 through SEQ ID NO: 19.

4. A Senolytic Peptide(s) comprising the amino acid sequence of one of claim 1, wherein the artificial peptide further comprises an N-terminal amino acid sequence that facilitates cellular uptake or a C-terminal amino acid sequence that facilitates cellular uptake.

5. The cell penetrating peptide according to of claim 1, wherein the cell penetrating peptide further comprises an N-terminus amino acid sequence comprising a single D-amino acid insertion.

6. The cell penetrating peptide according to claim 1, wherein the cell penetrating peptide exhibits a circular structure.

7. A method for treating a senescence-associated disease or disorder comprising administering to a subject in need thereof a therapeutically-effective amount of a peptide comprising an amino acid sequence having at least 70% identity to any one of SEQ ID NO: 1 through SEQ ID NO: 19 that selectively kill senescent cells over non-senescent cells; wherein said peptide is not used for intracellular delivery vehicle for another compound, wherein said disorder is selected from the group of age-related disorders consisting of atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness, wherein the method of administration of therapeutically-effective amount of said peptide is by any of three shock dose regimes namely Impulse Regime, Sustained Shock Regime, and Gentle Shock Regime, wherein each dose regime independently comprises a treatment course whose duration varies from 1 day to 2 weeks and followed by a non-treatment interval of at least 2 weeks.

8. The method of use of claim 7, wherein said therapeutically Effective Dose and said method of administration are as described in Schedule 1.

9. The method of use of claim 7, wherein said disorder is cancer, and wherein the use is for administration to a mammalian subject, preferably a human, before, during and/or after subjecting said subject to radiation therapy, and/or before, during or after administering to said subject at least one chemotherapeutic agent.

10. The method of use of claim 9, wherein the cancer is a therapy-resistant cancer.

11. The method of use in claim 10, wherein said therapy-resistant cancer is metastatic melanoma, breast cancer or glioblastoma, preferably metastatic melanoma, and wherein said therapy to which said cancer is resistant is radiation therapy or chemotherapy.

12. The method of use of claim 7, wherein said peptide is used for removing senescent cells in a human subject suffering from, or expected to suffer from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

13. The method of use of claim 7, wherein said peptide is used for removing cells that express p16INK4a in a subject suffering from, or expected to suffer from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

14. The method of use of claim 7, wherein said peptide is used for altering the levels of the Serine-46 phosphorylated p53 foci in a subject suffering, or expected to suffer, from atherosclerosis; chronic inflammatory diseases such as arthritis or arthrosis; cancer; osteoarthritis; glomerulosclerosis, diabetes including diabetes type I and II; diabetic ulcers; kyphosis; scoliosis; hepatic insufficiency; cirrhosis; Hutchinson-Gilford progeria syndrome (HGPS); laminopaties; osteoporosis; dementia; (cardio)vascular diseases; myocardial infarction; obesity; metabolic syndrome; acute myocardial infarction; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; renal insufficiency; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

15. A method for treating a senescence-associated disease or disorder comprising administering to a subject in need thereof a therapeutically-effective amount of a pharmaceutical compound which is any one of the senolytic iopromide compounds that selectively kill senescent cells over non-senescent cells; wherein iopromide is not used for image enhancement in radiology wherein said disorder is selected from the group of age-related disorders consisting of atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness, wherein the method of administration of therapeutically-effective amount of said compound is by any of three shock dose regimes namely Impulse Regime, Sustained Shock Regime, and Gentle Shock Regime, wherein each dose regime independently comprises a treatment course whose duration varies from 1 day to 2 weeks and followed by a non-treatment interval of at least 2 weeks.

16. The method of use of claim 15, wherein said disorder is cancer, and wherein the use is for administration to a mammalian subject, preferably a human, before, during and/or after subjecting said subject to radiation therapy, and/or before, during or after administering to said subject at least one chemotherapeutic agent.

17. The method of use claim 16, wherein the said cancer is a cancer resistant to therapy.

18. The method of use of claim 15, wherein said therapy-resistant cancer is metastatic melanoma, breast cancer or glioblastoma, preferably metastatic melanoma, and wherein said therapy to which said cancer is resistant is radiation therapy or chemotherapy.

19. The method of use of claim 15, wherein said compound is used for removing senescent cells in a human subject suffering from, or expected to suffer from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

20. The method of use of claim 15, wherein said compound is used for removing cells that express p16INK4a in a subject suffering from, or expected to suffer from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.

21. The method of use of claim 15, wherein said compound is used for for altering the levels of the Serine-46 phosphorylated p53 foci in a subject suffering, or expected to suffer, from atherosclerosis; chronic inflammatory diseases such as arthritis or arthrosis; cancer; osteoarthritis; glomerulosclerosis; diabetic ulcers; kyphosis; scoliosis; hepatic insufficiency; cirrhosis; Hutchinson-Gilford progeria syndrome (HGPS); laminopaties; osteoporosis; dementia; obesity; metabolic syndrome; emphysema; insulin sensitivity; boutonneuse fever; sarcopenia; neurodegenerative diseases such as Alzheimer's, Huntington's or Parkinson's disease; cataracts; anemia; hypertension; fibrosis; age-related macular degeneration; COPD; asthma; reducing or preventing graft failure after organ or tissue transplantation; ischemia reperfusion injury; incontinence; hearing loss such as deafness; vision loss such as blindness; sleeping disturbances; pain such as joint pain or leg pain; imbalance; fear; depression; breathlessness; weight loss; hair loss; muscle loss; loss of bone density; frailty and/or reduced fitness.
